(19) United States
(12) Patent Application Publication
(10) Pub. No.: US 2026/0265359 A1
(43) Pub. Date: Sep. 10, 2026

(54) MULTISPECIFIC FUSION PROTEINS TARGETING ANGIOGENIC AND INFLAMMATORY FACTORS

(71) Applicant: Hoffmann-La Roche Inc., Little Falls, NJ (US)

(72) Inventors: Wei Yong SHEN, Carlton (AU); Jinzhong ZHANG, Orinda, CA (US)

(21) Appl. No.: 19/562,784

(22) Filed: Mar. 11, 2026

Related U.S. Application Data
(63) Continuation of application No. PCT/CN2023/118036, filed on Sep. 11, 2023.

(51) Int. Cl. C07K 16/22; A61K 38/00; A61K 39/00; A61P 27/02; C07K 14/71; C07K 16/28

(52) U.S. Cl. CPC C07K 16/22; A61P 27/02; C07K 14/71; C07K 16/2866; A61K 38/00; A61K 2039/505; C07K 2317/569; C07K 2317/92; C07K 2319/00

(57) ABSTRACT

Provided is an antibody fusion protein comprising a binding construct targeting and being capable of binding Ang-2, IL-6 receptor, and at least one of the VEGF family members; or an antigen-binding fragment or domain thereof. The antibody fusion protein comprises, as binding components, an antibody or binding polypeptide directed against Ang-2, an antibody directed against IL-6R, and a plurality of extracellular domains of VEGF receptors. The antibody fusion protein is useful in treating or controlling an ocular or systemic disease, condition, or disorder that has etiology in aberrant angiogenesis, increased vascular leakage, or inflammation.

### CROSS REFERENCE TO RELATED APPLICATION

This application is bypass continuation of International Patent Application No. PCT/CN2023/118036, entitled “MULTISPECIFIC FUSION PROTEINS TARGETING ANGIOGENIC AND INFLAMMATORY FACTORS” and filed on Sep. 11, 2023 and is hereby incorporated by reference herein in its entirety.

### SEQUENCE LISTING

This application contains a Sequence Listing which has been submitted electronically in XML format and is hereby incorporated by reference in its entirety. Said XML copy, created on May 17, 2026 is named P60871_US_1_Sequence_Listing.xml and is 247,939 bytes in size

### BACKGROUND OF THE INVENTION

The present invention relates to multispecific fusion proteins targeting angiogenic and inflammatory factors. In particular, the present invention relates to multispecific fusion proteins targeting angiopoietin-2 (“Ang-2”), certain members of the family of vascular endothelial growth factors (“VEGF”), and interleukin-6 receptor (“IL-6R”). More particular, the present invention relates to multispecific antibody fusion proteins targeting Ang-2, VEGF-A, VEGF-B, PIGF, and IL-6R. The present invention also relates to such fusion proteins, their uses, and processes for production.

The major cellular components of the mammalian vascular system are the endothelium, smooth muscle cells, and pericytes. Endothelial cells form the lining of the inner surface of all blood vessels in the mammal and constitute a non-thrombogenic interface between blood and tissue. Therefore, the proliferation of endothelial cells is an important component for the development of new capillaries and blood vessels which, in turn, is a necessary process for the growth and/or regeneration of mammalian tissues.

In recent decades, a variety of signaling molecules have been identified as playing important roles in angiogenesis and increased vascular permeability (vascular leakage). These signaling molecules include members of the VEGF family (“VEGF family members”), angiopoietins, ephrin, Delta-like 4 ligand, and certain members of the interleukin family (such as IL-3, IL-6, IL-8, and IL-17). VEGF family members of secreted polypeptides have been shown to play an extremely important role in promoting endothelial cell proliferation and angiogenesis. A pathological feature of uncontrolled angiogenesis caused by VEGF over-expression is increased vascular permeability, which results in fluid leakage into, and swelling of, the surrounding tissues. In mammals, this family consists of five related growth factors having highly conserved receptor-binding structure: vascular endothelial growth factors A-D (“VEGF-A,” “VEGF-B,” “VEGF-C,” and “VEGF-D”) and placental growth factor (“PIGF”). In this disclosure, this family of growth factors is also referred to as the VEGF family.

The cytokine interleukin-6 (“IL-6”) plays an important role in host defense against environmental stress such as infection and injury. Under physiological conditions, IL-6 is barely detectable, but its levels can increase more than 100,000-fold during early phase of inflammation. However, not all occurrences of IL-6 stimulation are beneficial. Dysregulated, persistent production of IL-6 has been implicated in the development of various autoimmune and chronic inflammatory diseases. There has been evidence that unchecked production of IL-6 in inflamed tissues induces excess production of VEGF-A and VEGF-C.

The angiopoietin/Tie ligand/receptor system has a key regulatory role in regulating vascular integrity and quiescence. Besides its role in angiogenesis, it is an important regulator in numerous diseases including inflammation. Important members of the angiopoietin family are Ang-1 and Ang-2. Ang-1-mediated Tie-2 activation is required to maintain the quiescent resting state of the endothelium. Agonistic Ang-1 functions are antagonized by Ang-2, which is believed to inhibit Ang-1/Tie-2 signaling. Ang-2 destabilizes the quiescent endothelium and primes it to respond to exogenous stimuli, thereby facilitating the activities of inflammatory and angiogenic cytokines. It has been shown that Ang-2 promotes the proangiogenic action of VEGF and that VEGF up-regulates Ang-2 expression in endothelial cells.

The VEGF-family growth factors act through a family of cognate receptor tyrosine kinases, which exist only on the surface of vascular endothelial cells, to stimulate formation of blood vessels: VEGF receptor-1 (“VEGFR-1,” also known as “flt-1”), VEGF receptor-2 (“VEGFR-2,” also known as “KDR” in humans and “flk-1” in mice), VEGF receptor-3 (“VEGFR-3,” also known as “flt-4”).

VEGF-A (also sometimes simply referred to as VEGF) has emerged as the most important member of this family of growth factors. Human VEGF-A is expressed in a variety of tissues as multiple homodimeric forms (121, 145, 165, 183, 189 and 206 amino acids per monomer), wherein each form arises as a result of alternative splicing of a single RNA transcript.

Since VEGFs promote vascular endothelial cell proliferation and angiogenesis, they may be useful for the therapeutic treatment of numerous conditions in which a growth-promoting activity on the vascular endothelial cells is beneficially important; for example, in treatment of ulcers, vascular injuries, and myocardial infarction.

In contrast, however, while vascular endothelial proliferation is desirable under certain circumstances, vascular endothelial proliferation and angiogenesis are also undesirable components of a variety of diseases and disorders including tumor growth and metastasis, rheumatoid arthritis, psoriasis, atherosclerosis, diabetic retinopathy, retrolental fibroplasia, neovascular glaucoma, neovascular age-related macular degeneration, hemangiomas, immune rejection of transplanted corneal tissue and other tissues, and chronic inflammation. In individuals suffering from any of these disorders, one would want to inhibit, or at least substantially reduce, the endothelial proliferating activity of the aforementioned angiogenic factors.

Each of flt-1, KDR, and flt-4 tyrosine kinase receptors has seven extracellular immunoglobulin-like (“Ig-like”) domains that are available for ligand binding, a transmembrane domain that serves to anchor the receptor on the surface of cells in which it is expressed, and an intracellular catalytic tyrosine kinase domain. Flt-1 binds VEGF-A, VEGF-B, and PIGF. KDR binds VEGF-A, VEGF-C, and VEGF-D. Flt-4 binds VEGF-C and VEGF-D.

In view of the role of the growth factors of the VEGF family in vascular endothelial proliferation and angiogenesis, and the role that these processes play in many different diseases and disorders, treatments have been devised that target the control of these growth factors. However, anti-VEGF therapy alone has not been able to block completely the progression of angiogenic diseases.

Therefore, it is desirable to have a pharmacological means for more completely reducing or inhibiting one or more of the biological activities of these growth factors in patients whose pathological conditions are rooted in aberrant angiogenesis. It is also desirable to have a pharmacological means for improved treatment or control of pathological conditions that are rooted in aberrant angiogenesis.

### SUMMARY OF THE INVENTION

As used herein, the term “control” also includes reduction, alleviation, amelioration, or prevention.

In general, the present invention provides multispecific fusion or chimeric proteins, methods of producing and compositions comprising the same, and methods for treating or controlling at least a pathological condition in a subject, which condition has etiology in aberrant angiogenesis, increased vascular permeability (vascular leakage) and inflammation. In this disclosure, a “fusion protein” may be referred to as a “chimeric protein” for its inclusion of components from different origins.

In particular, a multispecific fusion protein of the present invention comprises an Ang-2-binding unit, an IL-6R-binding unit, and a VEGF-binding unit. An Ang-2-binding unit is a polypeptide or protein that is capable of binding, or substantially binding, to Ang-2. An IL-6R-binding unit is a polypeptide or protein that is capable of binding, or substantially binding, to IL-6R. A VEGF-binding unit is a polypeptide or protein that is capable of binding, or substantially binding, to one or more VEGF family members.

In one aspect, such multispecific fusion protein is a multispecific antibody fusion protein.

In another aspect, such multispecific antibody fusion protein is a trispecific fusion protein.

In still another aspect, the present invention provides trispecific antibody fusion or chimeric proteins, or antigen-binding fragments or antigen-binding domains thereof that are capable of binding substantially to Ang-2, IL-6R (including either the membrane-bound or the soluble form of IL-6R), and one or more VEGF family members; thereby, concurrently reducing or inhibiting Ang-2, IL-6, and VEGF family member signaling transduction.

In yet another aspect, a trispecific antibody fusion or chimeric protein of the present invention comprises Ang-2-, IL-6R-, and VEGF-binding units that are linked together. Such binding units comprise, consist of, or consist essentially of antigen-binding domains or moieties that target Ang-2, IL-6R, and one or more VEGF family members.

In still another aspect, a trispecific antibody fusion or chimeric protein of the present invention comprises a VEGF/IL-6R binding unit that comprises a VEGF-binding unit linked to an IL-6R-binding unit, and an Ang-2-binding unit linked to the VEGF/IL-6R binding unit.

The present invention also provides an antigen-binding fragment or domain of such fusion proteins.

In one aspect, the IL-6R-binding unit comprises an IL-6R antibody.

In another aspect, a trispecific antibody fusion protein of the present invention comprises an antibody against IL-6R, linked to an Ang-2-binding unit and a VEGF-binding unit. In yet another aspect, an Ang-2-binding unit comprises an antibody or a biologically active polypeptide that is capable of binding, or binding substantially, to Ang-2. In the present disclosure, the term “antibody” encompasses, without limitation, full-length antibodies, single-chain Fv antibodies (scFv), Fab antibodies, Fab′ antibodies, (Fab′) 2 antibodies, single-domain antibodies (sdAbs, also known as nanobodies), minibodies, maxibodies, diabodies, and peptibodies.

In still another aspect, a VEGF-binding unit included in a trispecific antibody fusion protein comprises an Ig-like domain selected from the group consisting of Ig-like domains of one or more VEGF receptors. In one aspect, VEGF family members that bind to such VEGF-binding unit include VEGF-A, VEGF-B, and PIGF. In another aspect, such family members are VEGF-A, VEGF-B, and PIGF. Thus, in one aspect, an antibody fusion protein of the present invention may be viewed at least as a trispecific construct that can bind to three distinct types of ligands that are involved in pathological angiogenesis and vascular leakage. In one aspect, said VEGF-binding unit comprises a plurality of Ig-like domains of one or more VEGF receptors. In some embodiments, the VEGF-binding unit comprises a plurality of Ig-like domains of VEGF receptors 1 and 2 (“VEGFR-1” and “VEGFR-2”). In some other embodiments, said VEGF-binding unit comprises Ig-like domain (extracellular domain) 2, or substantially Ig-like domain 2, of VEGFR-1 (“VEGFR-1-D2”) and Ig-like domain 3, or substantially domain 3, of VEGFR-2 (“VEGFR-2-D3”). In still some other embodiments, the VEGF-binding unit comprises VEGFR-1-D2 and VEGFR-2-D3 linked to an Fc domain of IgG1.

In still another aspect, said IL-6R is human IL-6R, said Ang-2 is human Ang-2, and said VEGFR-1 and VEGFR-2 are human VEGFR-1 and VEGFR-2.

In still another aspect, an antibody fusion or chimeric protein of the present invention, or an antigen-binding fragment or domain thereof, comprises an antibody against IL-6R linked to an Ang-2 binding unit and a VEGF-binding unit, wherein the antibody against IL-6R comprises full-length antibody or an sdAb directed against IL-6R.

In one aspect, the Ang-2-binding unit comprises an sdAb or a biologically active polypeptide directed against Ang-2.

In yet another aspect, a trispecific antibody fusion protein of the present invention comprises an Fc domain of human IgG1.

In still another aspect, said VEGF-binding unit comprises human VEGFR-1-D2 linked to human VEGFR-2-D3.

In yet another aspect, said VEGF-binding unit comprises: (a) human VEGFR-1-D2; (b) human VEGFR-2-D3; and (c) an Fc domain of IgG1; wherein the VEGFR-1-D2 and VEGFR-2-D3 are linked in series. In some embodiments, the C-terminus of VEGFR-2-D3 is linked to the N-terminus of the Fc domain. In other embodiments, the C-terminus of VEGFR-1-D2 is linked to the N-terminus of the Fc domain.

In yet another aspect, an sdAb against Ang-2 or IL-6R, or an antigen-binding fragment or domain thereof, comprises the heavy-chain variable region of a heavy-chain antibody against Ang-2 or IL-6R. Such an sdAb can bind specifically to Ang-2 or IL-6R without requiring a complementary variable region as in a conventional four-chain immunoglobulin molecule.

In still another aspect, the present invention provides an isolated nucleic acid molecule encoding an antibody fusion or chimeric protein.

In still another aspect, the present invention provides a vector that comprises said nucleic acid molecule, including an expression vector comprising said nucleic acid molecule operatively linked to an expression control sequence. As used herein, the phrase “operatively linked” refers to components of a construct being placed in a functional relationship with each other and each component retaining its function. A nucleic acid is “operatively linked” when it is placed in a functional relationship with another nucleic acid sequence. For example, DNA for a pre-sequence or secretory leader is “operatively linked” to DNA encoding a polypeptide if it is expressed as a pre-protein that participates in the secretion of the polypeptide; a promoter or enhancer is operatively linked to a coding sequence if it affects the transcription of the sequence; or a ribosome binding site is operatively linked to a coding sequence if it is positioned so as to facilitate translation.

In still another aspect, the present invention provides a host-vector system for the production of said antibody fusion or chimeric protein that comprises the expression vector in a suitable host cell.

In another aspect, the present invention provides a method of producing an antibody fusion or chimeric protein, which method comprises: (a) growing cells of the host-vector system under conditions permitting production of the antibody fusion or chimeric protein; and (b) recovering the antibody fusion or chimeric protein so produced. Such method can further comprise purifying the antibody fusion or chimeric protein.

In still another aspect, the present invention provides a method for treating or controlling, or a composition for use to treat or control, at least a disease, condition, or disorder, in a subject, which has etiology in a condition selected from the group consisting of aberrant angiogenesis, vascular leakage, inflammation, and combinations thereof.

In certain embodiments, such disease, condition, or disorder is an ocular disease, condition, or disorder. In certain other embodiments, such disease, condition, or disorder involves tumor growth and metastasis. In other embodiments, such disease, condition, or disorder is rheumatoid arthritis, psoriasis, or atherosclerosis.

Other features and advantages of the present invention will become apparent from the following detailed description and claims.

### BRIEF DESCRIPTION OF THE DRAWINGS

FIG. 1 shows a schematic diagram of the first embodiment of the present invention.

FIG. 2 shows a schematic diagram of the second embodiment of the present invention.

FIG. 3A and FIG. 3B shows schematic diagrams of the third embodiment of the present invention.

FIG. 4A and FIG. 4B shows schematic diagrams of the fourth embodiment of the present invention.

FIG. 5A and FIG. 5B_show the purity of antibody fusion proteins EB-105BI-7CA and EB-105BIc1, as exhibited by chromatograms of SEC-HPLC.

FIG. 6A to FIG. 6C show ELISA binding affinity of antibody fusion proteins of the present invention for human VEGF-A165.

FIG. 7 shows ELISA binding affinity of some antibody fusion proteins of the present invention for human VEGF-B167.

FIG. 8A and FIG. 8B show ELISA binding affinity of some antibody fusion proteins of the present invention for human PIGF.

FIG. 9A to FIG. 9C show ELISA binding affinity of some antibody fusion proteins of the present invention for human IL-6R.

FIG. 10A to FIG. 10C show ELISA binding affinity of some antibody fusion proteins of the present invention for human Ang-2.

FIG. 11 shows ELISA binding affinity of some antibody fusion proteins of the present invention, nesvacumab, and faricimab for human Ang-1.

FIG. 12A to FIG. 12C show inhibition of VEGF-A165-mediated VEGFR-2 signaling by antibody fusion proteins of the present invention.

FIG. 13A and FIG. 13B show inhibition of Ang-2/Tie-2 interaction by antibody fusion proteins of the present invention, nesvacumab, and faricimab.

FIG. 14A and FIG. 14B show inhibition of Ang-1/Tie-2 interaction by antibody fusion proteins of the present invention and nesvacumab.

FIG. 15A and FIG. 15B show blocking of binding of IL-6 to IL-6R by antibody fusion proteins of the present invention, tocilizumab, and vobarilizumab.

FIG. 16 shows the effect of fusion protein B21138002 on inhibiting vascular leakage from pre-retinal neovascularization (PRN) induced by DL-α-aminoadipic acid (DL-AAA) in Dutch belted rabbits.

FIG. 17 shows quantitative analysis of vascular leakage after intravitreal (IVT) injections of B21138002, aflibercept (Eylea), and faricimab in DL-AAA induced PRN in Dutch belted rabbits.

FIG. 18 shows the effect of B21138002, aflibercept, and faricimab on inhibiting vascular leakage from laser-induced choroidal neovascularization (CNV) in monkeys.

FIG. 19 shows quantitative analysis of changes in grade-IV CNV lesions after IVT injections of B21138002, aflibercept, and faricimab.

FIG. 20 shows quantitative analysis of vascular leakage in laser-induced CNV in monkeys after IVT injections of B21138002, aflibercept, and faricimab.

### DETAILED DESCRIPTION OF THE INVENTION

The terms “protein,” “polypeptide,” and “peptide” are used interchangeably herein to refer to a polymer of amino acid residues.

In general, the present invention provides a multispecific fusion or chimeric protein or an antigen-binding fragment or domain thereof that is capable of binding, or binding substantially, to Ang-2, IL-6R, and one or more VEGF family members; thereby, reducing or inhibiting Ang-2, IL-6, and VEGF family member signaling transduction.

In one aspect, the multispecific fusion or chimeric protein is an antibody fusion or chimeric protein.

In this disclosure, the term “antibody fusion protein” may be used in place of “antibody fusion or chimeric protein.”

In one aspect, an antibody fusion protein or an antigen-binding fragment thereof is a trispecific antibody fusion protein capable of binding, or substantially binding, to Ang-2, IL-6R (both soluble and membrane bound), and one or more VEGF family members; thereby, reducing or inhibiting signaling transduction of Ang-2, IL-6, and VEGF family members.

In another aspect, a multispecific fusion or chimeric protein of the present invention comprises binding units that target Ang-2, IL-6R, and one or more VEGF family members, and are linked together. Such binding units comprise, consist of, or consist essentially of antigen-binding domains or moieties that target Ang-2, IL-6R, and one or more VEGF family members. In yet another aspect, such a multispecific fusion or chimeric protein is a trispecific antibody fusion or chimeric protein.

In still another aspect, a trispecific antibody fusion protein of the present invention does not bind to Ang-1. In another aspect, a trispecific antibody fusion protein of the present invention has an affinity to Ang-1 that is less than about 10⁻³ times the affinity to Ang-2.

In another aspect, a trispecific antibody fusion protein of the present invention comprises an antibody against IL-6R, or an antigen-binding fragment or domain thereof, linked to an Ang-2-binding unit and a VEGF-binding unit.

In some embodiments, at least one of the VEGF-binding unit and the Ang-2-binding unit is linked directly to the antibody against IL-6R, or an antigen-binding fragment or domain thereof.

In some other embodiments, one of the VEGF-binding unit and the Ang-2-binding unit is linked to the antibody against IL-6R, or an antigen-binding fragment or domain thereof through an intervening polypeptide. Such an intervening polypeptide may comprise an IgG1 Fc domain.

In yet another aspect, the Ang-2-binding unit comprises an antigen-binding fragment of an antibody against Ang-2, or a biologically active peptide that binds to, or substantially binds to Ang-2.

In still another aspect, the VEGF-binding unit included in a trispecific antibody fusion protein comprises an Ig-like domain selected from the group consisting of Ig-like domains of one or more VEGF receptors. A VEGF-binding unit binds, or substantially binds, to at least one of VEGF family members. In one aspect, such VEGF family members include VEGF-A, VEGF-B, and PIGF. In another aspect, such family members are VEGF-A, VEGF-B, and PIGF. Thus, in one aspect, an antibody fusion protein of the present invention may be viewed at least as a trispecific construct that can bind to three distinct types of ligands that are involved in pathological angiogenesis and vascular leakage. In one aspect, said VEGF-binding unit comprises a plurality of Ig-like domains of one or more VEGF receptors. In some embodiments, said VEGF-binding unit comprises a plurality of Ig-like domains of VEGFR-1 and VEGFR-2. In some other embodiments, said VEGF-binding unit comprises VEGFR-1-D2 and VEGFR-2-D3.

In still another aspect, said IL-6R is human IL-6R, said Ang-2 is human Ang-2, and said VEGFR-1 and VEGFR-2 are human VEGFR-1 and VEGFR-2.

In still another aspect, an antibody fusion or chimeric protein of the present invention, or an antigen-binding fragment or domain thereof, comprises an antibody against IL-6R linked to an Ang-2-binding unit and a VEGF-binding unit; wherein the antibody against IL-6R comprises a full-length antibody, an IL-6R-binding fragment thereof, or an sdAb directed against IL-6R. In some embodiments, an IL-6R-binding antibody fragment comprises complementarity determining regions (“CDRs”) of the heavy chain and the light chain of an IL-6R antibody. In some other embodiments, an IL-6R-binding antibody fragment comprises fewer than all CDRs of the heavy chain and the light chain of an IL-6R antibody.

In one aspect, the binding unit that targets Ang-2 comprises an sdAb or a biologically active polypeptide directed against Ang-2.

In yet another aspect, a trispecific antibody fusion protein of the present invention comprises an Fc domain of human IgG1.

In still another aspect, said VEGF-binding unit comprises human VEGFR-1-D2 linked to human VEGFR-2-D3, directly or through a peptide linker. Such a peptide linker, when used, is preferably a short peptide linker, such as having fewer than 20 amino acid residues. Peptide linkers are known in the art, such as peptides comprising glycine, serine, and/or threonine residues. Common peptide linkers comprise short sequences of glycine and serine residues. The amino acid sequences of human VEGFR-1-D2 and VEGFR-2-D3 are shown below as SEQ ID NO: 1 and SEQ ID NO: 2.

In yet another aspect, said VEGF-binding unit comprises: (a) human VEGFR-1-D2; (b) human VEGFR-2-D3; and (c) an Fc domain of IgG1; wherein the VEGFR-1-D2 and VEGFR-2-D3 are linked in series. In some embodiments, the C-terminus of VEGFR-2-D3 is linked to the N-terminus of the Fc domain. In other embodiments, the C-terminus of VEGFR-1-D2 is linked to the N-terminus of the Fc domain. The amino acid sequence of Fc domain of human IgG1 is shown below as SEQ ID NO: 3.

In yet another aspect, an sdAb against Ang-2 or IL-6R, or an antigen-binding fragment or domain thereof, comprises the heavy-chain variable region of a heavy-chain antibody (VHH) against Ang-2 or IL-6R. Such an sdAb can bind specifically to Ang-2 or IL-6R without requiring a complementary variable region as in a conventional four-chain immunoglobulin molecule. An sdAb directed against Ang-2 is sometimes denoted herein as “Ang-2 sdAb.” An sdAb directed against IL-6R is sometimes denoted herein as “IL-6R sdAb.”

As disclosed herein, an “antigen-binding fragment or domain” of an antibody refers to a fragment or portion of such antibody, which fragment or portion is capable of binding, or binding substantially, to the antigen. In one embodiment, an antigen-binding fragment or domain of such antibody comprises, consists essentially of, or consists of a variable domain of the heavy chain (VH) of such antibody.

An sdAb directed against Ang-2 or IL-6R of included in some embodiments of the present invention consists of three CDRs of an antibody heavy chain, each CDR being flanked by framework domains. Such sdAb lacks the CH1 domain of the antibody heavy chain. Despite possessing only three CDRs, sdAbs show equivalent antigen-binding affinity and other effector functions compared to conventional antibodies comprising six CDRs (three CDRs of the heavy chain and three CDRs of the light chain). Single-domain antibodies are described, for example, in Bathula et al., Cancer Biotherapy and Radiopharmaceuticals, Vol. 36, No. 2, 109-122 (2021).

An sdAb directed against Ang-2 or IL-6R included in embodiments of the present invention is capable of binding to its corresponding ligand (Ang-2 or IL-6R) with an equilibrium dissociation constant (K_{D}) in the range from about 1×10⁻⁶ M to 1×10⁻¹² M. In some embodiments, the sdAb against Ang-2 or IL-6R is capable of binding to its corresponding ligand with K_{D} in the range from about 1×10⁻⁷ M to about 1×10⁻¹² M. In some other embodiments, the sdAb against Ang-2 or IL-6R is capable of binding to its corresponding ligand with K_{D} in the range from about 1×10⁻⁸ M to about 1×10⁻¹² M. In still some embodiments, the sdAb against Ang-2 or IL-6R is capable of binding to its corresponding ligand with K_{D} in the range from about 1×10⁻⁹ M to about 1×10⁻¹² M.

### First Embodiment of the Trispecific Fusion Proteins of the Present Invention

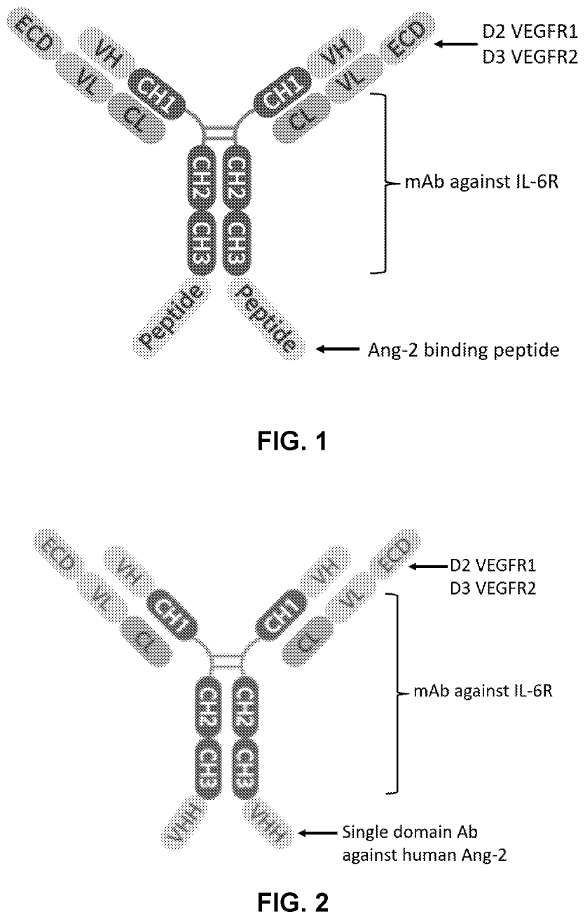

In one aspect, a first embodiment of the trispecific fusion proteins of the present invention comprises: (a) an IL-6R-binding unit that comprises an antibody against IL6-R (“IL-6R antibody”) comprising a heavy chain and a light chain; (b) a VEGF-binding unit comprising VEGFR-1-D2 and VEGFR-2-D3, linked together in series; and (c) a polypeptide capable of binding Ang-2 (“Ang-2-binding polypeptide”); wherein a C-terminus of the VEGF-binding unit is linked to an N-terminus of the light chain of the IL-6R antibody; and a C-terminus of the heavy chain of the IL-6R antibody is linked to an N-terminus of the Ang-2-binding polypeptide. See FIG. 1. Alternatively, the C-terminus of the VEGF-binding unit is linked to the N-terminus of the heavy chain of the IL-6R antibody. In one aspect, the C-terminus of the heavy chain of the IL-6R antibody is the C-terminus of the Fc domain of an IgG1.

In another aspect, the IL-6R-binding unit of the first embodiment of the trispecific fusion proteins of the present invention comprises: (1) the CDR1, CDR2, and CDR3 of a heavy chain of an IL-6R antibody linked to at least one of CH2 and CH3 of the Fc domain of IgG1; and (2) the CDR1, CDR2, and CDR3 of the light chain of the IL-6R antibody.

Flexible linkers having various lengths may be used to link an Ang-2-binding polypeptide to the C-terminus of the Fc domain of the heavy chain of the IL-6R antibody. Non-limiting examples of peptide linkers may be those including or consisting of a motif of (GGGGS)ₓ (x=1, 2, 3, 4, 5, 6, 7, 8, 9 or 10). Specific examples of such flexible linkers are GGGGSGGGGSGGGGS, GGGGSGGGGS, and GGGGSGGGS.

The amino acid sequences disclosed or claimed herein also encompass conservative amino acid substitutions in these sequences, which do not generally alter the biological activity of the proteins or peptides. The most commonly occurring substitutions are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions.

Non-limiting examples of an IL-6R antibody are tocilizumab (described in; e.g., U.S. Pat. Nos. 10,323,095; 7,479,543; and 5,795,965), sarilumab (described in; e.g., U.S. Pat. No. 7,582,298), and satralizumab (described in; e.g., U.S. Pat. No. 8,562,991). The amino acid sequences of the heavy and light chains of tocilizumab are shown in SEQ ID NO: 4 and SEQ ID NO: 5. Additional non-limiting examples of IL-6R antibodies are disclosed in U.S. Pat. No. 8,753,634 (to Apexigen, Inc.) and U.S. Pat. No. 8,748,581 (to Ablynx N.V.). The foregoing patents are incorporated herein by reference to the extent of the disclosure of the IL-6R antibodies.

Non-limiting examples of an Ang-2-binding peptides are shown in SEQ ID NO: 6 through SEQ ID NO: 11. Other Ang-2-binding polypeptides that may be used to construct a fusion protein of the first embodiment are disclosed, for example, in U.S. Pat. Nos. 7,138,370 and 7,205,275, which are incorporated herein by reference to the extent of the disclosure of the Ang-2-binding polypeptides.

Nine trispecific fusion proteins were constructed (denoted as EB-105BI-7CA, EB-105BI-7CB, EB-105BI-10C, EB-105BI-15CA, EB-105BI-15CB, EB-105BI-21C, EB-105BI-Con4C, EB-105BI-Con4-40CA, and EB-105BI-Con4-40CB). Each fusion protein comprises: (a) tocilizumab (an IL-6R antibody) comprising a heavy chain and a light chain; (b) a VEGF-binding unit comprising VEGFR-1-D2 and VEGFR-2-D3, linked in series in that order; and (c) an Ang-2-binding polypeptide selected from the group consisting of SEQ ID NO: 6 through SEQ ID NO: 11; wherein the C-terminus of the VEGF-binding unit is linked to the N-terminus of the light chain of tocilizumab; and the C-terminus of the heavy chain of tocilizumab is linked to the N-terminus of the Ang-2-binding polypeptide.

Complete sequences of the heavy chain and the light chain of each of these fusion proteins are disclosed in SEQ ID NO: 12 through SEQ ID NO: 29.

Other trispecific fusion proteins having the same binding units may be constructed, wherein the C-terminus of the VEGF-binding unit is linked to the N-terminus of the heavy chain of tocilizumab.

Still other trispecific fusion proteins of the first embodiment may be constructed, wherein the IL-6R antibody comprises sarilumab or satralizumab.

Still other trispecific fusion proteins of the first embodiment may be constructed, wherein the IL-6R-binding unit comprises: (1) the CDR1, CDR2, and CDR3 of the heavy chain (“HCCDR1,” “HCCDR2,” and “HCCDR3”) of tocilizumab (shown as SEQ ID NO: 63, 64, and 65, respectively) linked to at least one of CH2 and CH3 of the Fc domain of IgG1; and (2) the CDR1, CDR2, and CDR3 of the light chain (“LCCDR1,” LCCDR2,” and “LCCDR3”) of tocilizumab (shown as SEQ ID NO: 66, 67, and 68, respectively).

Still other trispecific fusion proteins of the first embodiment may be constructed, wherein the IL-6R-binding unit comprises: (1) the HCCDR1, HCCDR2, and HCCDR3 of sarilumab or satralizumab linked to at least one of CH2 and CH3 of the Fc domain of IgG1; and (2) the LCCDR1, LCCDR2, and LCCDR3 of sarilumab or satralizumab.

### Second Embodiment of the Trispecific Fusion Proteins of the Present Invention

In one aspect, a second embodiment of the trispecific fusion proteins of the present invention comprises: (a) an IL-6R-binding unit that comprises an IL-6R antibody comprising a heavy chain and a light chain; (b) a VEGF-binding unit comprising VEGFR-1-D2 and VEGFR-2-D3, linked in series; and (c) an sdAb (VHH) directed against Ang-2 (“Ang-2 sdAb”); wherein a C-terminus of the VEGF-binding unit is linked to an N-terminus of the light chain of the IL-6R antibody; and a C-terminus of the heavy chain of the IL-6R antibody is linked to an N-terminus of the Ang-2 sdAb. See FIG. 2. Alternatively, the C-terminus of the VEGF-binding unit is linked to the N-terminus of the heavy chain of the IL-6R antibody.

In another aspect, the IL-6R-binding unit comprises: (1) the CDR1, CDR2, and CDR3 of a heavy chain of an IL-6R antibody linked to at least one of CH2 and CH3 of the Fc domain of IgG1; and (2) the CDR1, CDR2, and CDR3 of the light chain of the IL-6R antibody.

One of the flexible linkers disclosed herein above may be used to link the N-terminus of the Ang-2 sdAb to the C-terminus of the Fc domain of the IL-6R antibody.

Non-limiting examples of IL-6R antibodies are disclosed herein above.

The present inventors generated Ang-2 sdAbs in a discovery program, the amino acid sequences of eight of which are shown in SEQ ID NO: 30 through SEQ ID NO: 37. Other Ang-2 sdAbs that may be used to construct a fusion protein of the second embodiment are disclosed, for example, in U.S. Pat. No. 9,527,925 (to Boehringer Ingelheim GmbH), which is incorporated herein by reference to the extent of the disclosure of the Ang-2 sdAbs.

Trispecific fusion proteins of the second embodiment were constructed. Each fusion protein comprises: (a) tocilizumab (an IL-6R antibody) comprising a heavy chain and a light chain; (b) a VEGF-binding unit comprising VEGFR-1-D2 and VEGFR-2-D3, linked in series in that order; and (c) an Ang-2 sdAb having an amino acid sequence selected from the group consisting of SEQ ID NO: 30 through SEQ ID NO: 37; wherein the C-terminus of the VEGF-binding unit is linked to the N-terminus of the light chain of tocilizumab; and the C-terminus of the heavy chain of tocilizumab is linked to the N-terminus of the Ang-2 sdAb.

Two such fusion proteins were constructed (denoted as EB-105BI-c1 and EB-105BI-c2) that comprise Ang-2 sdAbs having SEQ ID NO: 31 and SEQ ID NO: 32, respectively.

Complete amino acid sequences of the heavy chains and the light chains of EB-105BI-c1 and EB-105BI-c2 are shown in SEQ ID NO: 38 through SEQ ID NO: 41.

Other trispecific fusion proteins of the second embodiment may be constructed, wherein the IL-6R antibody comprises sarilumab or satralizumab.

Still other trispecific fusion proteins of the second embodiment may be constructed, wherein the IL-6R-binding unit comprises: (1) the HCCDR1, HCCDR2, and HCCDR3 of tocilizumab linked to at least one of CH2 and CH3 of the Fc domain of IgG1; and (2) the LCCDR1, LCCDR2, and LCCDR3 of tocilizumab.

Still other trispecific fusion proteins of the second embodiment may be constructed, wherein the IL-6R-binding unit comprises: (1) the HCCDR1, HCCDR2, and HCCDR3 of sarilumab or satralizumab linked to at least one of CH2 and CH3 of the Fc domain of IgG1; and (2) the LCCDR1, LCCDR2, and LCCDR3 of sarilumab or satralizumab.

### Third Embodiment of the Fusion Proteins of the Present Invention

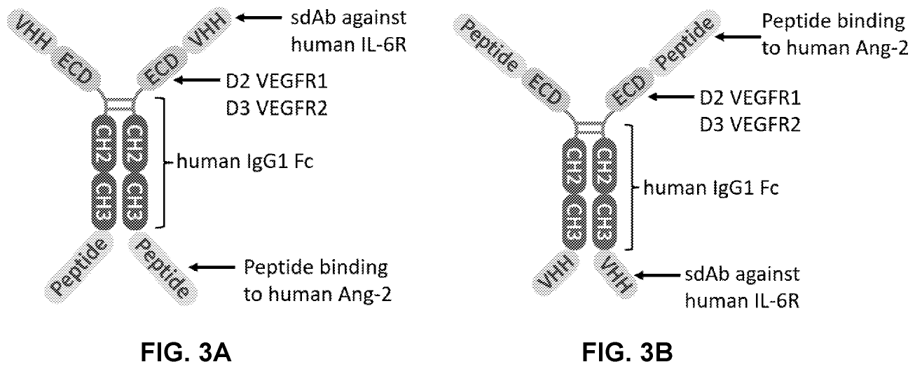

In one aspect, a third embodiment of the trispecific fusion proteins of the present invention comprises two fusion polypeptides, each of which comprises: (a) an sdAb directed against IL-6R (“IL-6R sdAb”); (b) a VEGF-binding unit comprising VEGFR-1-D2 and VEGFR-2-D3, linked in series; (c) an Ang-2-binding polypeptide; and (d) an Fc domain of IgG1; wherein a C-terminus of the VEGF-binding unit is linked to an N-terminus of the Fc domain, the N-terminus of the VEGF-binding unit is linked to the C-terminus of the IL-6R sdAb, and the N-terminus of the Ang-2-binding polypeptide is linked to the C-terminus of the Fc domain. See FIG. 3A. The N-terminus of the VEGF-binding unit is linked to the C-terminus of the IL-6R sdAb directly or through a flexible linker. The N-terminus of the Ang-2-binding polypeptide is linked to the C-terminus of the Fc domain directly or through a flexible linker. Flexible linkers disclosed herein above, or other flexible linkers known in the art, may be used.

In another aspect, a third embodiment of the trispecific fusion proteins of the present invention comprises two fusion polypeptides, each of which comprises: (a) IL-6R sdAb; (b) a VEGF-binding unit comprising VEGFR-1-D2 and VEGFR-2-D3, linked in series; (c) an Ang-2-binding polypeptide; and (d) an Fc domain of IgG1; wherein a C-terminus of the VEGF-binding unit is linked to an N-terminus of the Fc domain, the N-terminus of the VEGF-binding unit is linked to the C-terminus of the Ang-2-binding polypeptide, and the N-terminus of the IL-6R sdAb is linked to the C-terminus of the Fc domain. See FIG. 3B. The N-terminus of the VEGF-binding unit is linked to the C-terminus of the Ang-2-binding polypeptide directly or through a flexible linker. The N-terminus of the IL-6R sdAb is linked to the C-terminus of the Fc domain directly or through a flexible linker. Flexible linkers disclosed herein may be used.

The IL-6R sdAbs included in the two fusion polypeptides may be the same or different. The Ang-2-binding polypeptides included in the two fusion polypeptides may be the same or different.

The Ang-2 polypeptides disclosed herein above, including SEQ ID NO: 6 through SEQ ID NO: 11, may be used to construct a fusion protein of the third embodiment.

The present inventors generated IL-6R sdAbs in a discovery program, the amino acid sequences of fifteen of which are shown in SEQ ID NO: 42 through SEQ ID NO: 56. Other IL-6R sdAbs that may be used to construct a fusion protein of the third embodiment are disclosed, for example, in U.S. Pat. No. 10,618,964 (assigned to Ablynx N.V.); U.S. Pat. No. 8,753,634 (assigned to Apexigen, Inc.); and U.S. Pat. No. 8,748,581 (assigned to Ablynx N.V.), which are incorporated herein by reference to the extent of the disclosure of the IL-6R sdAbs.

Two trispecific fusion proteins (denoted as EB-105BId1 and EB-105BId3) of the third embodiment were constructed. The fusion protein comprises two fusion polypeptides, each comprising: (a) IL-6R sdAb having SEQ ID NO: 42 (included in EB-105BId1) or SEQ ID NO: 44 (included in EB-105BId3); (b) a VEGF-binding unit comprising VEGFR-1-D2 (SEQ ID NO: 1) and VEGFR-2-D3 (SEQ ID NO: 2), linked in series; (c) Ang-2-binding polypeptide having SEQ ID NO: 6; and (d) an Fc domain of IgG1 (SEQ ID NO: 3); wherein the C-terminus of the VEGF-binding unit is linked to an N-terminus of the Fc domain, the N-terminus of the VEGF-binding unit is linked to the C-terminus of the IL-6R sdAb, and the N-terminus of the Ang-2-binding polypeptide is linked to the C-terminus of the Fc domain. The N-terminus of the VEGF-binding unit is linked to the C-terminus of the IL-6R sdAb through a flexible linker. The N-terminus of the Ang-2-binding polypeptide is linked to the C-terminus of the Fc domain through a flexible linker.

The complete amino acid sequences of antibody fusion proteins EB-105BId1 and EB-105BId3 are shown in SEQ ID NO: 57 and SEQ ID NO: 58, respectively.

Two trispecific fusion proteins of the present invention (denoted as EB-105BId2 and EB-105BId4) were constructed. The fusion protein comprises two fusion polypeptides, each of which comprises: (a) IL-6R sdAb having SEQ ID NO: 42 (included in EB-105BId2) or SEQ ID NO: 44 (included in EB-105BId4); (b) a VEGF-binding unit comprising VEGFR-1-D2 (SEQ ID NO: 1) and VEGFR-2-D3 (SEQ ID NO: 2), linked in series; (c) Ang-2-binding polypeptide having SEQ ID NO: 6; and (d) an Fc domain of IgG1 (SEQ ID NO: 3); wherein a C-terminus of the VEGF-binding unit is linked to an N-terminus of the Fc domain, the N-terminus of the VEGF-binding unit is linked to the C-terminus of the Ang-2-binding polypeptide, and the N-terminus of the IL-6R sdAb is linked to the C-terminus of the Fc domain. The N-terminus of the VEGF-binding unit is linked to the C-terminus of the Ang-2-binding polypeptide through a flexible linker. The N-terminus of the IL-6R sdAb is linked to the C-terminus of the Fc domain through a flexible linker.

The complete amino acid sequences of antibody fusion proteins EB-105BId2 and EB-105BId4 are shown in SEQ ID NO: 59 and SEQ ID NO: 60, respectively.

### Fourth Embodiment of the Fusion Proteins of the Present Invention

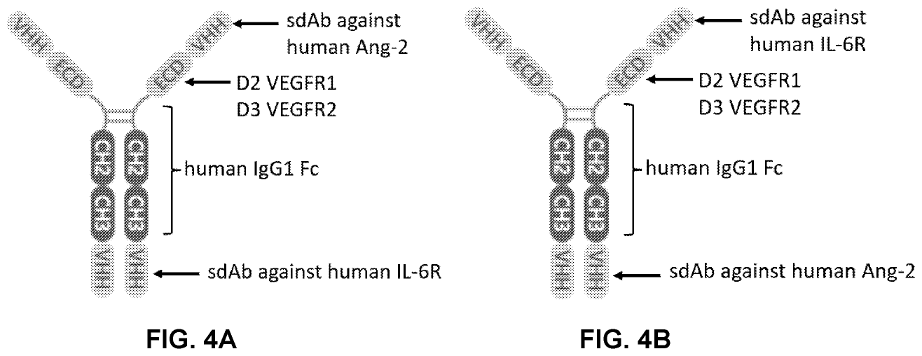

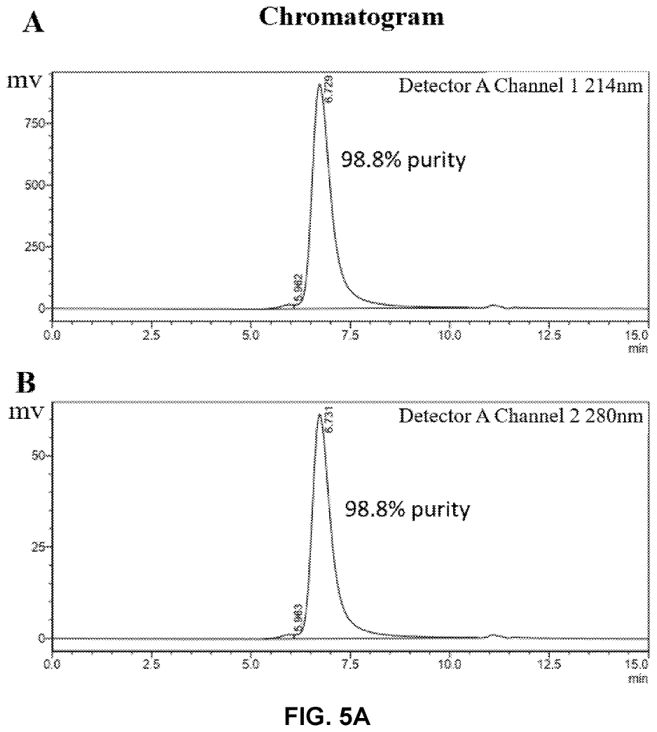

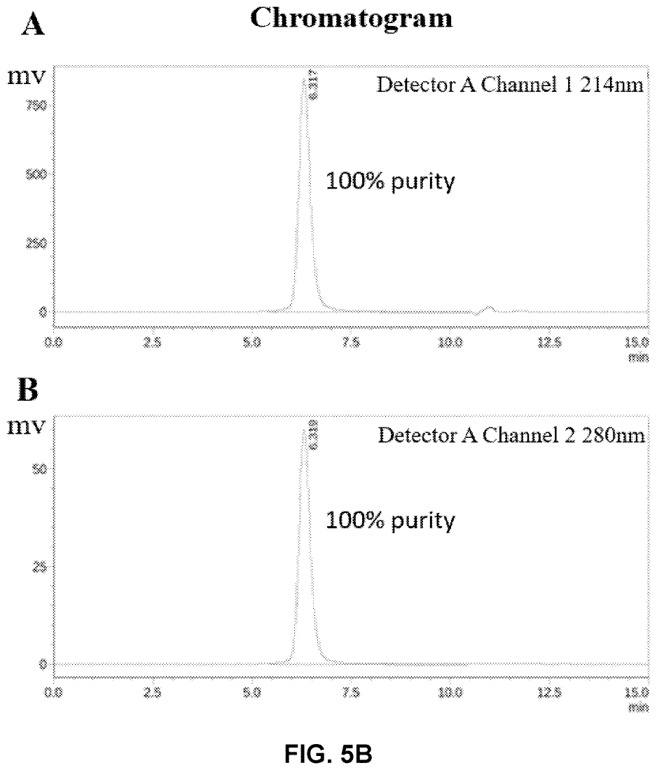

In one aspect, a fourth embodiment of the trispecific fusion proteins of the present invention comprises two fusion polypeptides, each of which comprises: (a) an IL-6R sdAb; (b) a VEGF-binding unit comprising VEGFR-1-D2 and VEGFR-2-D3, linked in series; (c) an Ang-2 sdAb; and (d) an Fc domain of IgG1; wherein a C-terminus of the VEGF-binding unit is linked to an N-terminus of the Fc domain, the N-terminus of the VEGF-binding unit is linked to the C-terminus of the Ang-2 sdAb, and the N-terminus of the IL-6R sdAb is linked to the C-terminus of the Fc domain. See FIG. 4A. The N-terminus of the VEGF-binding unit is linked to the C-terminus of the Ang-2 sdAb directly or through a flexible linker. The N-terminus of the IL-6R sdAb is linked to the C-terminus of the Fc domain directly or through a flexible linker. Flexible linkers disclosed herein above, or other flexible linkers known in the art, may be used.

In another aspect, a fourth embodiment of the trispecific fusion proteins of the present invention comprises two fusion polypeptides, each of which comprises: (a) an IL-6R sdAb; (b) a VEGF-binding unit comprising VEGFR-1-D2 and VEGFR-2-D3, linked in series; (c) an Ang-2 sdAb; and (d) an Fc domain of IgG1; wherein a C-terminus of the VEGF-binding unit is linked to an N-terminus of the Fc domain, the N-terminus of the VEGF-binding unit is linked to the C-terminus of the IL-6R sdAb, and the N-terminus of the Ang-2 sdAb is linked to the C-terminus of the Fc domain. See FIG. 4B. The N-terminus of the VEGF-binding unit is linked to the C-terminus of the IL-6R sdAb directly or through a flexible linker. The N-terminus of the Ang-2 sdAb is linked to the C-terminus of the Fc domain directly or through a flexible linker. Flexible linkers disclosed herein above may be used.

The IL-6R sdAbs included in the two fusion polypeptides may be the same or different. The Ang-2 sdAbs included in the two fusion polypeptides may be the same or different.

The Ang-2 sdAbs disclosed herein above, including SEQ ID NO: 30 through SEQ ID NO: 37, may be used to construct a fusion protein of the fourth embodiment.

The IL-6R sdAbs disclosed herein above, including SEQ ID NO: 42 through SEQ ID NO: 56, may be used to construct a fusion protein of the fourth embodiment.

A trispecific fusion protein of the fourth embodiment (denoted as EB-105BIe1) was constructed. The fusion protein comprises two fusion polypeptides, each comprising: (a) IL-6R sdAb having SEQ ID NO: 42; (b) a VEGF-binding unit comprising VEGFR-1-D2 (SEQ ID NO: 1) and VEGFR-2-D3 (SEQ ID NO: 2), linked in series; (c) Ang-2 sdAb having SEQ ID NO: 31; and (d) an Fc domain of IgG1 (SEQ ID NO: 3); wherein the C-terminus of the VEGF-binding unit is linked to an N-terminus of the Fc domain, the N-terminus of the VEGF-binding unit is linked to the C-terminus of the Ang-2 sdAb, and the N-terminus of the IL-6R sdAb is linked to the C-terminus of the Fc domain. The N-terminus of the VEGF-binding unit is linked to the C-terminus of the Ang-2 sdAb through a flexible linker. The N-terminus of the IL-6R sdAb is linked to the C-terminus of the Fc domain through a flexible linker.

The complete amino acid sequence of antibody fusion protein EB-105BIe1 is shown in SEQ ID NO: 61.

Another trispecific fusion protein of the fourth embodiment (denoted as EB-105BIe2) was constructed. The fusion protein comprises two fusion polypeptides, each comprising: (a) IL-6R sdAb having SEQ ID NO: 42; (b) a VEGF-binding unit comprising VEGFR-1-D2 (SEQ ID NO: 1) and VEGFR-2-D3 (SEQ ID NO: 2), linked in series; (c) Ang-2 sdAb having SEQ ID NO: 31; and (d) an Fc domain of IgG1 (SEQ ID NO: 3); wherein the C-terminus of the VEGF-binding unit is linked to an N-terminus of the Fc domain, the N-terminus of the VEGF-binding unit is linked to the C-terminus of the IL-6R sdAb, and the N-terminus of the Ang-2 sdAb is linked to the C-terminus of the Fc domain. The N-terminus of the VEGF-binding unit is linked to the C-terminus of the IL-6R sdAb through a flexible linker. The N-terminus of the Ang-2 sdAb is linked to the C-terminus of the Fc domain through a flexible linker.

The complete amino acid sequence of antibody fusion protein EB-105BIe2 is shown in SEQ ID NO: 62.

An IL-6R-binding unit included in a trispecific antibody fusion protein of the present invention is capable of binding IL-6R with K_{D} in the range from about 1×10⁻⁶ M to about 1×10⁻¹² M. In some embodiments, said K_{D} is in the range from about 1×10⁻⁷ M to about 1×10⁻¹² M. In some other embodiments, said K_{D} in the range from about 1×10⁻⁸ M to about 1×10⁻¹² M. In still some embodiments, said K_{D} is in the range from about 1×10⁻⁹ M to about 1×10⁻¹² M. As a result, said antibody fusion protein substantially inhibits biological activity of IL-6R in promoting angiogenesis and inflammation; thereby, controlling a pathological condition having etiology in aberrant angiogenesis and inflammation.

An Ang-2-binding unit included in a trispecific antibody fusion protein of the present invention is capable of binding Ang-2 with K_{D} in the range from about 1×10⁻⁶ M to about 1×10⁻¹² M. In some embodiments, said K_{D} is in the range from about 1×10⁻⁷ M to about 1×10⁻¹² M. In some other embodiments, said K_{D} in the range from about 1×10⁻⁸ M to about 1×10⁻¹² M. In still some embodiments, said K_{D} is in the range from about 1×10⁻⁹ M to about 1×10⁻¹² M. As a result, said antibody fusion protein substantially inhibits biological activity of Ang-2 in promoting angiogenesis and increased vascular permeability; thereby, controlling a pathological condition having etiology in aberrant angiogenesis and vascular leakage.

A VEGF-binding unit included in a trispecific antibody fusion protein of the present invention is capable of binding at least one of VEGF-A, VEGF-B, and PIGF with K_{D} in the range from about 1×10⁻⁶ M to about 1×10⁻¹² M. In some embodiments, said K_{D} is in the range from about 1×10⁻⁷ M to about 1×10⁻¹² M. In some other embodiments, said K_{D} in the range from about 1×10⁻⁸ M to about 1×10⁻¹² M. In still some other embodiments, said K_{D} is in the range from about 1×10⁻⁹ M to about 1×10⁻¹² M. As a result, said antibody fusion protein substantially inhibits biological activity of said at least one of said VEGF family members in promoting angiogenesis; thereby, controlling a pathological condition having etiology in aberrant angiogenesis and vascular leakage.

In some embodiments, the present invention also provides a binding construct that comprises, consists of, or consists essentially of a plurality of trispecific antibody fusion or chimeric proteins, herein described, that are linked to or associated with each other by covalent bonds or other forms of attachment; wherein the trispecific antibody fusion proteins of such a binding construct may be the same or different. Such a binding construct of the present invention is capable of binding Ang-2, IL-6R, and at least one of VEGF-A, VEGF-B, and PIGF with high affinity. In the case where the trispecific antibody fusion proteins are different, each can comprise a different binding unit against Ang-2, IL-6R, or VEGF family members, selected from the binding units disclosed herein.

A trispecific antibody fusion protein or a binding construct may further include a heterologous peptide or other chemical moieties. Such additions can modify its properties such as stability, solubility, toxicity, serum half-life, immunogenicity, detectability, or other properties.

The term “high affinity” is used in a physiological context pertaining to the relative affinity of the trispecific antibody fusion protein for Ang-2, IL-6R, and said VEGF family members in vivo in a mammal, such as a laboratory test animal, a domesticated farm or pet animal, or a human. Trispecific antibody fusion proteins binding Ang-2, IL-6R, and said VEGF family members in the present invention can have characteristic affinities for their ligands in vivo, typically measured in terms of sub-nanomolar values of equilibrium dissociation constants (K_{D}). For the purposes of this invention, a trispecific antibody fusion protein of the present invention can bind to its targeted ligand with a K_{D} less than or equal to about 1, or about 5, or about 10, or about 50, or about 100, or about 500, or about 1000 times the K_{D} of the natural ligand/receptor pair.

A trispecific antibody fusion proteins of the present invention is capable of binding to Ang-2, IL-6R, and at least a VEGF family member with an equilibrium dissociation constant (K_{D}) in the range from about 1×10⁻⁶ M to 1×10⁻¹² M. In some embodiments, the K_{D} value is in the range from about 1×10⁻⁷ M to about 1×10⁻¹² M, or from about 1×10⁻⁸ M to about 1×10⁻¹² M, or from about 1×10⁻⁹ M to about 1×10⁻¹² M.

In another aspect, a trispecific antibody fusion protein may comprise more than one of each of the Ang-2-binding unit, the VEGF-binding unit, and the IL-6R-binding unit.

In one aspect, the amino acid sequences of the non-limiting various portions or embodiments of a trispecific antibody fusion protein of the present invention are listed in Table 1.

TABLE 1
Amino Acid Sequences

| Portion or Embodiment of a | SEQ ID |
|---|---|
| Trispecific Antibody Fusion Protein | NO: |
| Human VEGFR-1-D2 | 1 |
| Human VEGFR-2-D3 | 2 |
| Human IgG1 Fc domain | 3 |
| Tocilizumab heavy chain | 4 |
| Tocilizumab light chain | 5 |
| Ang-2-binding Peptide 1 | 6 |
| Ang-2-binding Peptide 2 | 7 |
| Ang-2-binding Peptide 3 | 8 |
| Ang-2-binding Peptide 4 | 9 |
| Ang-2-binding Peptide 5 | 10 |
| Ang-2-binding Peptide 6 | 11 |
| EB-105BI-7CA heavy chain | 12 |
| EB-105BI-7CA light chain | 13 |
| EB-105BI-7CB heavy chain | 14 |
| EB-105BI-7CB light chain | 15 |
| EB-105BI-10C heavy chain | 16 |
| EB-105BI-10C light chain | 17 |
| EB-105BI-15CA heavy chain | 18 |
| EB-105BI-15CA light chain | 19 |
| EB-105BI-15CB heavy chain | 20 |
| EB-105BI-15CB light chain | 21 |
| EB-105BI-21C heavy chain | 22 |
| EB-105BI-21C light chain | 23 |
| EB-105BI-Con4C heavy chain | 24 |
| EB-105BI-Con4C light chain | 25 |
| EB-105BI-Con4C-40CA heavy chain | 26 |
| EB-105BI-Con4C-40CA light chain | 27 |
| EB-105BI-Con4C-40CB heavy chain | 28 |
| EB-105BI-Con4C-40CB light chain | 29 |
| Ang-2 sdAb A38-051 | 30 |
| Ang-2 sdAb A38-196 | 31 |
| Ang-2 sdAb A41-089 | 32 |
| Ang-2 sdAb A41-093 | 33 |
| Ang-2 sdAb A16-022 | 34 |
| Ang-2 sdAb A16-030 | 35 |
| Ang-2 sdAb A38-053 | 36 |
| Ang-2 sdAb A41-121 | 37 |
| EB-105BIcl heavy chain | 38 |
| EB-105BIcl light chain | 39 |
| EB-105BIc2 heavy chain | 40 |
| EB-105BIc2 light chain | 41 |
| IL-6R sdAb N_PM 119 | 42 |
| IL-6R sdAb N PM 123 | 43 |
| IL-6R sdAb N PM 180 | 44 |
| IL-6R sdAb N_PM_184 | 45 |
| IL-6R sdAb H PM A5 108 008 | 46 |
| IL-6R sdAb H_PM_A5_108_031 | 47 |
| IL-6R sdAb H PM A5 108 040 | 48 |
| IL-6R sdAb H_PM_A5_108_046 | 49 |
| IL-6R sdAb H PM A5 108 058 | 50 |
| IL-6R sdAb C82-111-Fc-011 | 51 |
| IL-6R sdAb C82-111-Fc-013 | 52 |
| IL-6R sdAb C82-111-Fc-020 | 53 |
| IL-6R sdAb C82-111-Fc-033 | 54 |
| IL-6R sdAb C82-111-Fc-043 | 55 |
| IL-6R sdAb C82-111-Fc-059 | 56 |
| EB-105BId1 | 57 |
| EB-105BId3 | 58 |
| EB-105BId2 | 59 |
| EB-105BId4 | 60 |
| EB-105BIel | 61 |
| EB-105BIe2 | 62 |
| Tocilizumab heavy chain CDR1 | 63 |
| Tocilizumab heavy chain CDR2 | 64 |
| Tocilizumab heavy chain CDR3 | 65 |
| Tocilizumab light chain CDR1 | 66 |
| Tocilizumab light chain CDR2 | 67 |
| Tocilizumab light chain CDR3 | 68 |
| Sarilumab heavy chain | 69 |
| Sarilumab light chain | 70 |
| Satralizumab heavy chain | 71 |
| Satralizumab light chain | 72 |

In another aspect, the nucleic acid sequences encoding the amino acid sequences of Table 1 are listed in Table 2.

TABLE 2
Nucleic Acid Sequences

| Portion or Embodiment of a | SEQ ID |
|---|---|
| Trispecific Antibody Fusion Protein | NO: |
| Human VEGFR-1-D2 | 73 |
| Human VEGFR-2-D3 | 74 |
| Human IgG1 Fc domain | 75 |
| Tocilizumab heavy chain | 76 |
| Tocilizumab light chain | 77 |
| Ang-2-binding Peptide 1 | 78 |
| Ang-2-binding Peptide 2 | 79 |
| Ang-2-binding Peptide 3 | 80 |
| Ang-2-binding Peptide 4 | 81 |
| Ang-2-binding Peptide 5 | 82 |
| Ang-2-binding Peptide 6 | 83 |
| EB-105BI-7CA heavy chain | 84 |
| EB-105BI-7CA light chain | 85 |
| EB-105BI-7CB heavy chain | 86 |
| EB-105BI-7CB light chain | 87 |
| EB-105BI-10C heavy chain | 88 |
| EB-105BI-10C light chain | 89 |
| EB-105BI-15CA heavy chain | 90 |
| EB-105BI-15CA light chain | 91 |
| EB-105BI-15CB heavy chain | 92 |
| EB-105BI-15CB light chain | 93 |
| EB-105BI-21C heavy chain | 94 |
| EB-105BI-21C light chain | 95 |
| EB-105BI-Con4C heavy chain | 96 |
| EB-105BI-Con4C light chain | 97 |
| EB-105BI-Con4C-40CA heavy chain | 98 |
| EB-105BI-Con4C-40CA light chain | 99 |
| EB-105BI-Con4C-40CB heavy chain | 100 |
| EB-105BI-Con4C-40CB light chain | 101 |
| Ang-2 sdAb A38-051 | 102 |
| Ang-2 sdAb A38-196 | 103 |
| Ang-2 sdAb A41-089 | 104 |
| Ang-2 sdAb A41-093 | 105 |
| Ang-2 sdAb A16-022 | 106 |
| Ang-2 sdAb A16-030 | 107 |
| Ang-2 sdAb A38-053 | 108 |
| Ang-2 sdAb A41-121 | 109 |
| EB-105BIcl heavy chain | 110 |
| EB-105BIcl light chain | 111 |
| EB-105BIc2 heavy chain | 112 |
| EB-105BIc2 light chain | 113 |
| IL-6R sdAb N PM 119 | 114 |
| IL-6R sdAb N_PM 123 | 115 |
| IL-6R sdAb N PM 180 | 116 |
| IL-6R sdAb N PM 184 | 117 |
| IL-6R sdAb H PM_A5_108_008 | 118 |
| IL-6R sdAb H_PM_A5_108_031 | 119 |
| IL-6R sdAb H PM A5 108 040 | 120 |
| IL-6R sdAb H PM A5 108 046 | 121 |
| IL-6R sdAb H PM A5 108 058 | 122 |
| IL-6R sdAb C82-111-Fc-011 | 123 |
| IL-6R sdAb C82-111-Fc-013 | 124 |
| IL-6R sdAb C82-111-Fc-020 | 125 |
| IL-6R sdAb C82-111-Fc-033 | 126 |
| IL-6R sdAb C82-111-Fc-043 | 127 |
| IL-6R sdAb C82-111-Fc-059 | 128 |
| EB-105BId1 | 129 |
| EB-105BId3 | 130 |
| EB-105BId2 | 131 |
| EB-105BId4 | 132 |
| EB-105BIel | 133 |
| EB-105BIe2 | 134 |
| Tocilizumab heavy chain CDR1 | 135 |
| Tocilizumab heavy chain CDR2 | 136 |
| Tocilizumab heavy chain CDR3 | 137 |
| Tocilizumab light chain CDR1 | 138 |
| Tocilizumab light chain CDR2 | 139 |
| Tocilizumab light chain CDR3 | 140 |

In yet another aspect, a trispecific antibody fusion or chimeric protein of the present invention comprises an amino acid sequence that is at least 90% identical to any one of SEQ ID NO: 1 through SEQ ID NO: 72.

In still another aspect, a trispecific antibody fusion or chimeric protein of the present invention comprises an amino acid sequence that is at least 95% identical to any one of SEQ ID NO: 1 through SEQ ID NO: 72.

In still another aspect, the present invention provides a trispecific fusion protein comprising polypeptides having a pair of amino acid sequences selected from the group consisting of SEQ ID NOs: 12 and 13, 14 and 15, 16 and 17, 18 and 19, 20 and 21, 22 and 23, 24 and 25, 26 and 27, 28 and 29, 38 and 39, and 40 and 41.

In still another aspect, the present invention provides a trispecific fusion protein comprising a polypeptide having an amino acid sequence selected from the group consisting of SEQ ID NOs: 57-62.

In still another aspect, one or more amino acid substitutions can be made in any one of the above-described amino acid sequences. Preferably, such substitution is a conservative substitution, wherein an amino acid in one of the following groups is substituted with another in the same group: (1) A, G; (2) D, E; (3) N, Q; (4)R, K; (5) I, L, M, V; (6) F, Y, W; (7) S, T; and (8) C, M; and such substitution is selected so as to preserve substantially the binding activity of the fusion protein. In one embodiment, a trispecific antibody fusion protein of the present invention having a conservative substitution has a K_{D} value for Ang-2, IL-6R, VEGF-A, VEGF-B, or PIGF ligand less than about 120% of that before such substitution. Preferably, the K_{D} value is less than about 110% of that before such substitution. More preferably, the K_{D} value is less than about 105% of that before such substitution. Still more preferably, the K_{D} value is less than about 100% of that before such substitution.

In addition, the amino acid sequences disclosed or claimed herein also encompass their “conservatively modified variants,” which are the results of a substitution, deletion, or addition of a single amino acid or a small percentage of amino acids (such as ≤5, ≤4, ≤3, ≤2, or ≤1%) in the original sequence of a peptide, polypeptide, or protein sequence that does not substantially alter the biological activity of the original peptide, polypeptide, or protein. For example, such conservatively modified variants can retain about ≥95, ≥96, ≥97, ≥98, ≥99, or 100% of the biological activity of the original peptide, polypeptide, or protein.

Most conservative substitutions are not expected to produce radical changes in the characteristics of the Ig-like domain or other domains of the fusion polypeptide. However, when it is difficult to predict the exact effect of the substitution in advance of doing so, one skilled in the art will appreciate that the effect can be evaluated by routine screening assays. For example, an Ig-like domain or other domain variant typically is made by site-specific mutagenesis of the nucleic acid encoding the intact fusion polypeptide, expression of the variant nucleic acid in recombinant cell culture, purification of the variant fusion polypeptide from the cell culture, and detecting the ability of the variant fusion polypeptide to specifically bind to Ang-2, IL-6R, or an aforementioned VEGF ligand. An exemplary binding assay which can be employed to determine if a particular substitution or substitutions in an Ig-like domain or other domains affect the capability of the fusion polypeptide to bind to and inhibit the activity of Ang-2, IL-6R, or an aforementioned VEGF family member is described in the article by Park et al., J. Biol. Chem., 269:25646-25654 (1994).

The VEGFR-1-D2 binding unit of the fusion protein is capable of binding free VEGF-A, VEGF-B, and PIGF with high affinity (Davis-Smyth et al., EMBO J., 15(18): 4919 (1996)). The VEGFR-2-D3 binding unit of the fusion protein is capable of binding free VEGF-A, VEGF-C, and VEGF-D with high affinity (Stuttfeld et al., Life, 61(9): 915 (2009)). The Ang-2 and IL-6R binding units are capable of inhibiting the activation of Tie-2 by Ang-2, and IL-6R by IL-6, respectively. Thus, a fusion protein of the present invention is capable of substantially inhibiting the angiogenic activity of these growth factors on endothelial cells at the site of the disease.

In still another aspect, the present invention provides isolated nucleic acid molecules encoding said trispecific antibody fusion proteins.

In yet another aspect, the present invention provides isolated nucleic acid molecules encoding a trispecific antibody fusion protein; wherein said isolated nucleic acid molecules comprise: (a) a nucleic acid sequence encoding an IL-6R-binding unit comprising an IL-6R antibody; (b) a nucleic acid sequence encoding a VEGF-binding unit comprising VEGFR-1-D2-VEGFR-2-D3 operatively linked to said nucleic acid sequence encoding said IL-6R binding unit; and (c) a nucleic acid sequence encoding an Ang-2-binding polypeptide operatively linked to said nucleic acid sequence encoding said IL-6R-binding unit.

In yet another aspect, the present invention provides isolated nucleic acid molecules encoding a trispecific antibody fusion protein of the first embodiment disclosed herein above.

In still another aspect, the present invention provides isolated nucleic acid molecules encoding said trispecific antibody fusion protein; wherein said isolated nucleic acid molecules comprise: (a) nucleic acid sequences encoding the heavy chain and light chain of tocilizumab, having a sequence listed in SEQ ID NO: 76 and SEQ ID NO: 77; (b) a nucleic acid sequence encoding a VEGF-binding unit comprising VEGFR-1-D2-VEGFR-2-D3, having sequences listed in SEQ ID NO: 73 and SEQ ID NO: 74, which is operatively linked to the 5′ end of the nucleic acid sequence encoding the light chain of tocilizumab; and (c) a nucleic acid sequence encoding an Ang-2-binding peptide, having a sequence selected from the group consisting of SEQ ID NOs: 78-83, which is operatively linked to the 3′ end of the nucleic acid sequence encoding the heavy chain of tocilizumab.

In yet another aspect, the present invention provides isolated nucleic acid molecules encoding a trispecific antibody fusion protein; wherein said isolated nucleic acid molecules comprise: (a) a nucleic acid sequence encoding an IL-6R-binding unit comprising an IL-6R antibody; (b) a nucleic acid sequence encoding a VEGF-binding unit comprising VEGFR-1-D2-VEGFR-2-D3 operatively linked to said nucleic acid sequence encoding said IL-6R binding unit; and (c) a nucleic acid sequence encoding an Ang-2 sdAb operatively linked to said nucleic acid sequence encoding said IL-6R-binding unit. In one embodiment, the 3′ end of the nucleic acid sequence encoding the VEGF-binding unit is linked to the 5′ end of the nucleic acid sequence encoding the light chain of the IL-6R antibody, and the 5′ end of the nucleic acid sequence encoding the Ang-2 sdAb is linked to the 3′ end of the nucleic acid sequence encoding the heavy chain of the IL-6R antibody.

In yet another aspect, the present invention provides isolated nucleic acid molecules encoding a trispecific antibody fusion protein of the second embodiment disclosed herein above.

In still another aspect, the present invention provides isolated nucleic acid molecules encoding said trispecific antibody fusion protein; wherein said isolated nucleic acid molecules comprise: (a) nucleic acid sequences encoding the heavy chain and light chain of tocilizumab, having a sequence listed in SEQ ID NO: 76 and SEQ ID NO: 77; (b) a nucleic acid sequence encoding a VEGF-binding unit comprising VEGFR-1-D2-VEGFR-2-D3, having sequences listed in SEQ ID NO: 73 and SEQ ID NO: 74, which is operatively linked to the 5′ end of the nucleic acid sequence encoding the light chain of tocilizumab; and (c) a nucleic acid sequence encoding an Ang-2 sdAb, having a sequence selected from the group consisting of SEQ ID NOs: 102-109, which is operatively linked to 3′ end of the nucleic acid sequence encoding the heavy chain of tocilizumab.

In still another aspect, the present invention provides isolated nucleic acid molecules encoding a trispecific antibody fusion protein of the third embodiment; wherein said isolated nucleic acid molecules comprise: (a) a nucleic acid sequence encoding an IL-6R sdAb, having a sequence selected from the group consisting of SEQ ID NOs: 114-128; (b) a nucleic acid sequence encoding a VEGF-binding unit comprising VEGFR-1-D2 and VEGFR-2-D3, linked in series, having sequences listed in SEQ ID NOs: 73 and 74; (c) a nucleic acid sequence encoding an Ang-2-binding polypeptide, having a sequence selected from the group consisting of SEQ ID NOs: 78-83; and (d) a nucleic acid sequence encoding an Fc domain of IgG1; wherein the 3′ end of the nucleic acid sequence encoding the VEGF-binding unit is operatively linked to the N-terminus of the nucleic acid of the Fc domain, the 5′ end of the nucleic acid sequence encoding the VEGF-binding unit is linked operatively to the 3′ end of the nucleic acid sequence encoding the IL-6R sdAb, and the 5′ end of the nucleic acid sequence encoding the Ang-2-binding polypeptide is linked operatively to the 3′ end of the nucleic acid sequence encoding the Fc domain.

In still another aspect, the present invention provides isolated nucleic acid molecules encoding another trispecific antibody fusion protein of the third embodiment; wherein said isolated nucleic acid molecules comprise: (a) a nucleic acid sequence encoding an IL-6R sdAb, selected from the group consisting of SEQ ID NOs: 114-128; (b) a nucleic acid sequence encoding a VEGF-binding unit comprising VEGFR-1-D2 and VEGFR-2-D3, linked in series, having sequences listed in SEQ ID NOs: 73 and 74; (c) a nucleic acid sequence encoding an Ang-2-binding polypeptide, selected from the group consisting of SEQ ID NOs: 78-83; and (d) a nucleic acid sequence encoding an Fc domain of IgG1 listed in SEQ ID NO: 75; wherein the 3′ end of the nucleic acid sequence encoding the VEGF-binding unit is operatively linked to the 5′ end of the nucleic acid encoding the Fc domain, the 5′ end of the nucleic acid sequence encoding the VEGF-binding unit is linked operatively to the 3′ end of the nucleic acid sequence encoding the Ang-2-binding polypeptide, and the 5′ end of the nucleic acid sequence encoding the IL-6R sdAb is linked operatively to the 3′ end of the nucleic acid sequence encoding the Fc domain.

In another aspect, the present invention provides an isolated nucleic acid molecule encoding one of two chains of a trispecific antibody fusion protein of the fourth embodiment; wherein said isolated nucleic acid molecule comprises: (a) a nucleic acid sequence encoding an IL-6R sdAb selected from the group consisting of SEQ ID NOs: 114-128; (b) a nucleic acid sequence encoding a VEGF-binding unit comprising VEGFR-1-D2 and VEGFR-2-D3, linked in series, having sequences listed in SEQ ID NOs: 73 and 74; (c) a nucleic acid sequence encoding an Ang-2 sdAb, selected from the group consisting of SEQ ID NOs: 102-109; and (d) a nucleic acid sequence encoding an Fc domain of IgG1 listed in SEQ ID NO: 75; wherein the 3′ end of the nucleic acid sequence encoding the VEGF-binding unit is operatively linked to the 5′ end of the nucleic acid sequence encoding the Fc domain, the 5′ end of the nucleic acid sequence encoding the VEGF-binding unit is operatively linked to the 3′ end of the nucleic acid encoding the Ang-2 sdAb, and the 5′ end of the nucleic acid sequence encoding of the IL-6R sdAb is operatively linked to the 3′ end of the nucleic acid encoding the Fc domain.

In still another aspect, the present invention provides an isolated nucleic acid molecule encoding one of two chains of another trispecific antibody fusion protein of the fourth embodiment; wherein said isolated nucleic acid molecule comprises: (a) a nucleic acid sequence encoding an IL-6R sdAb selected from the group consisting of SEQ ID NOs: 114-128; (b) a nucleic acid sequence encoding a VEGF-binding unit comprising VEGFR-1-D2 and VEGFR-2-D3, linked in series, having sequences listed in SEQ ID NOs: 73 and 74; (c) a nucleic acid sequence encoding an Ang-2 sdAb, selected from the group consisting of SEQ ID NOs: 102-109; and (d) a nucleic acid sequence encoding an Fc domain of IgG1 listed in SEQ ID NO: 75; wherein the 3′ end of the nucleic acid sequence encoding the VEGF-binding unit is operatively linked to the 5′ end of the nucleic acid sequence encoding the Fc domain, the 5′ end of the nucleic acid sequence encoding the VEGF-binding unit is operatively linked to the 3′ end of the nucleic acid encoding the IL-6R sdAb, and the 5′ end of the nucleic acid sequence encoding of the Ang-2 sdAb is operatively linked to the 3′ end of the nucleic acid encoding the Fc domain.

In another aspect, the present invention provides isolated nucleic acid molecules encoding a trispecific antibody fusion or chimeric protein of the present invention; wherein said isolated nucleic acid molecules comprise a nucleic acid sequence that, as a result of the degeneracy of the genetic code, differs in one or more codons from a nucleic acid sequence listed in this disclosure. Such different nucleic acid sequence is within the scope of the present invention.

In still another aspect, the present invention provides a vector that comprises any of the nucleic acid molecules herein disclosed, including an expression vector comprising any of said nucleic acid molecules operatively linked to an expression control sequence.

In yet another aspect, a vector comprises a nucleic acid sequence encoding a trispecific antibody fusion protein, as listed in SEQ ID NOs: 84-101, 110-113, and 129-134.

In still another aspect, the present invention provides a host-vector system for the production of any of said trispecific antibody fusion or chimeric proteins, which host-vector system comprises the expression vector in a suitable host cell.

In one aspect, the present invention provides for the construction of a nucleic acid molecule encoding a trispecific antibody fusion protein disclosed herein, which nucleic acid molecule is inserted into a vector that is able to express the antibody fusion protein when introduced into an appropriate host cell. Appropriate host cells include, but are not limited to, bacterial cells, yeast cells, insect cells, and mammalian cells. Any of the methods known to one skilled in the art for the insertion of DNA fragments into a vector may be used to construct expression vectors encoding chimeric polypeptide molecules under control of transcriptional/translational control signals. These methods may include in vitro recombinant DNA and synthetic techniques and in vivo recombination (genetic recombination) (See; e.g., Sambrook, et al., Molecular Cloning, A Laboratory Manual, Cold Spring Harbor Laboratory; Current Protocols in Molecular Biology, Eds. Ausubel, et al., Greene Publ. Assoc., Wiley-Interscience, NY).

Expression of nucleic acid molecules encoding an antibody fusion protein of the present invention may be regulated by a second nucleic acid sequence (a promoter) so that the antibody fusion protein is expressed in a host transformed with the nucleic acid molecules. For example, expression of an antibody fusion protein described herein may be controlled by any promoter/enhancer element known in the art.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is typically transformed using pBR322, a plasmid derived from an E. coli species (see; e.g., Bolivar et al., Gene, 2:95 (1977)). The plasmid pBR322 contains genes for ampicillin and tetracycline resistance and thus provides easy means for identifying transformed cells. The pBR322 plasmid, or other microbial plasmid or phage, must also contain, or be modified to contain, promoters that can be used by the microbial organism for expression of proteins.

Those promoters most commonly used in recombinant DNA construction include the β-lactamase (penicillinase) and lactose promoter systems or a tryptophan (trp) promoter system (Goeddel et al., Nucleic Acids Res., 8:4057 (1980)). While these are the most commonly used, other microbial promoters have been discovered and utilized. For example, the tac promoter is a synthetically produced DNA promoter produced from the combination of promoters from the trp and lac operons (de Boer et al., PNAS, (1983-01-80 (1): 21-25 (1983)). It is commonly used for protein production in Escherichia coli. (Amann et al., Gene, 25:167 (1983)). Any of these promoters may be used in connection with a method of producing an antibody fusion protein of the present invention.

In addition to prokaryotes, eukaryotic microbes, such as yeast cultures, may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979)) is commonly used. Other exemplary plasmids are disclosed in U.S. Pat. No. 4,615,974; Struhl et al., PNAS, 76 (3): 1035 (1979). The plasmid YRp7 contains the trp1 gene that provides a selection marker for a mutant strain of yeast lacking the ability to grow without tryptophan, for example, ATCC No. 44,076 or RH218 (Jones, Genetics, 85:23 (1977)). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzeman et al., J. Biol. Chem., 255:2073 (1980)) or other glycolytic enzymes, such as glyceraldehyde-3-phosphate dehydrogenase, hexokinase, pyruvate decarboxylas, and glucokinase (Romanos et al., Yeast, 8:423 (1992); Weinhandl et al., Microb. Cell Factories, 13:5 (2014)). In constructing suitable expression plasmids, the termination sequences associated with these genes are also ligated into the expression vector 3′ of the sequence desired to be expressed to provide polyadenylation of the mRNA and termination. Other promoters, which have the additional advantage of transcription controlled by growth conditions, such as the promoter region for alcohol dehydrogenase 2, and enzymes responsible for maltose and galactose utilization (Romanos et al., Weinhandl et al., supra), may be used for the vector construction. Any plasmid vector containing yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, much interest has been in vertebrate cells, and propagation of vertebrate cells in culture (tissue culture) has become a routine procedure in recent years. Examples of such useful host cell lines are VERO and HeLa cells, Chinese hamster ovary (CHO) cell lines, and W138, BHK, COS-7, HEK293, and MDCK cell lines. Expression vectors for such cells ordinarily include (if necessary) an origin of replication, a promoter located in front of the gene to be expressed, along with any necessary ribosome binding sites, RNA splice sites, polyadenylation sites, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40). The early and late promoters of SV40 virus are particularly useful because both are obtained easily from the virus as a fragment that also contains the SV40 viral origin of replication (Fiers et al., Nature, 273:113 (1978)). Smaller or larger SV40 fragments may also be used, provided there is included the approximately 250-bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

Thus, according to the invention, expression vectors capable of being replicated in a bacterial, a yeast cell, an insect cell, or a mammalian cell host, comprising an antibody fusion protein-encoding nucleic acid as described herein, are used to transfect the host and thereby direct expression of such nucleic acids to produce the fusion polypeptide, which may then be recovered in a biologically active form. As used herein, a biologically active form includes a form capable of binding to at least a VEGF family member.

In some embodiments, the host cell can be E. coli, a COS cell, a HEK 293 cell (also known simply as 293 cells), or a Chinese hamster ovary (“CHO”) cell. Preferably, the host cell is a HEK 293 or CHO cell.

A non-limiting example is the plasmid pcDNA3.4, which is suitable to be used in a mammalian host cell, such as the CHO cell. The plasmid pcDNA3.4 contains genes for ampicillin resistance and genes for SV40 and CMV promoters.

### Vector Construction

Construction of suitable vectors containing the desired coding and control sequences employ standard ligation techniques. Isolated plasmids or DNA fragments are cleaved, tailored, and ligated in the form desired to form the plasmids required. The methods employed are not dependent on the DNA source or intended host. Cleavage is performed by treating with restriction enzyme (or enzymes) in suitable buffer.

A nucleic acid sequence substantially encoding one or more Ig-like domains of VEGFR-1 or VEGFR-2 can be produced according to the method disclosed in U.S. Pat. No. 6,897,294.

In a first embodiment, a nucleic acid sequence substantially encoding the Ig-like domain 2 of VEGFR-1 and the Ig-like domain 3 of VEGFR-2 is ligated in tandem in the desired order. This construct is then ligated to the 5′ end of the nucleic acid sequence encoding the light chain of an IL-6R antibody (e.g., tocilizumab). A nucleic acid sequence encoding an Ang-2-binding polypeptide is ligated to the 3′ end of the nucleic acid sequence encoding the heavy chain of the IL-6R antibody. Such entire nucleic acid sequences are referred to as a chimeric nucleic acid sequence.

The entire chimeric nucleic acid sequences are then positioned in a vector which contains a promoter in the reading frame with the gene and compatible with the proposed host cell. A number of plasmids, such as those described in U.S. Pat. Nos. 4,456,748; 5,460,811; 5,888,808; and 6,333,147 may be used in a production of an antibody fusion protein of the present invention.

In one aspect, the vector system pcDNA3.4 is suitable to express an antibody fusion protein of the present invention in a mammalian cell.

In one embodiment, an antibody fusion protein of the present invention may be produced according to the method described in U.S. Pat. No. 7,070,959. For example, the chimeric nucleic acid sequences of SEQ ID NOs: 84 and 85 are inserted into the expression vector pcDNA3.4 having the CMV promoter.

In one embodiment, CHO cells are transfected with pcDNA3.4/SEQ ID NOs: 84 and 85. The antibody fusion protein obtained from the CHO cells may be purified and characterized by binding assay, as described in U.S. Pat. No. 7,070,959.

Similarly, a nucleic acid molecule encoding another trispecific antibody fusion protein described herein above may be produced by ligating nucleic acid sequences encoding the various desired ligand-binding units in a desired order and then inserting into the expression vector pcDNA3.4. CHO cells are transfected with such vector and grown. Antibody fusion proteins obtained from these CHO cells may be similarly purified and characterized.

In one embodiment, an antibody fusion protein of the present invention can bind to human Ang-2 (“hAng-2”), human IL-6R (“hIL-6R”), and VEGF family members with K_{D}≤10⁻⁹M. In another embodiment, an antibody fusion protein of the present invention can bind to hAng-2, hIL-6R, and VEGF family members with K_{D}≤5×10⁻¹⁰ M. In still another embodiment, an antibody fusion protein of the present invention can bind to hAng-2, hIL-6R, and VEGF family members with Kp≤10-10 M.

In one aspect, the present invention provides compounds, compositions, and methods for treating or controlling a disease, condition, or disorder having etiology in aberrant angiogenesis and inflammation.

In another aspect, the present invention provides a method for treating or controlling at least an ocular or systemic disease, condition, or disorder, in a subject, which has etiology in aberrant angiogenesis and inflammation. The method comprises administering to a subject in need of such treating or controlling a composition comprising a trispecific antibody fusion protein herein disclosed. Non-limiting embodiments of such an antibody fusion protein have amino acid sequences listed in SEQ ID NOs: 12-29, 38-41, and 57-62.

In still another aspect, the present invention provides a composition for use to treat or control at least an ocular or systemic disease, condition, or disorder, in a subject, which has etiology in aberrant angiogenesis and inflammation, wherein composition comprises a trispecific antibody fusion protein herein disclosed. Non-limiting embodiments of such an antibody fusion protein have amino acid sequences listed in SEQ ID NOs: 12-29, 38-41, and 57-62.

In still another aspect, the present invention provides a use of a trispecific antibody fusion protein herein disclosed for the preparation of a pharmaceutical composition or a medicament for the treatment or control of at least an ocular or systemic disease, condition, or disorder, in a subject, which has etiology in aberrant angiogenesis and inflammation. Non-limiting embodiments of such an antibody fusion protein have amino acid sequences listed in SEQ ID NOs: 12-29, 38-41, and 57-62

In still another aspect, said ocular disease, condition, or disorder is selected from the group consisting of: macular edema resulting from diabetes, uveitis, central and branch retinal vein occlusion, choroidal neovascularization, neovascular age-related macular degeneration (wet age-related macular degeneration), polypoidal choroidal vasculopathy (“PCV”), myopic choroidal neovascularization, vascular leak, non-proliferative and proliferative diabetic retinopathy, retinopathy of prematurity, corneal neovascularization, corneal inflammation, and neovascular glaucoma.

In one embodiment, the subject is administered with a dose of about 25-4000 micrograms of the fusion protein. In another embodiment, the subject is administered with a dose of about 50-8000, about 100-8000, about 500-8000, about 1000-8000, about 2000-8000, about 50-6000, about 50-5000, about 50-4000, about 50-3000, about 50-2000, or about 50-1000 micrograms of the fusion protein.

In still another aspect, the composition comprising the fusion protein is in the form of an eye drop or an ocular injection (such as intravitreal, intracameral, peri-orbital, subtenon, subretinal, or suprachoroidal injection). Such a composition comprises an ophthalmic composition. An antibody fusion protein of the present invention may also be incorporated in a medical device that is implantable into or near a diseased tissue.

In one embodiment, the present invention provides a method for treating or controlling, or a composition for use to treat or control, an anterior-segment disease, condition, or disorder; such as corneal neovascularization, corneal inflammation, or neovascular glaucoma. The composition comprising the fusion protein may be in the form of an eye drop or intracameral or subconjunctival injection. In another embodiment, the present invention provides a method or composition for treating or controlling a posterior-segment disease, condition, or disorder; such as choroidal neovascularization, neovascular age-related macular degeneration (wet age-related macular degeneration), polypoidal choroidal vasculopathy (“PCV”), myopic choroidal neovascularization, vascular leak, macular edema resulting from diabetes, uveitis, central and branch retinal vein occlusion, non-proliferative and proliferative diabetic retinopathy, retinopathy of prematurity. In this case, the composition comprising the fusion protein may be administered in the form of an intravitreal injection.

In yet another aspect, an eye drop is administered to the subject at least once per day, at least once per week, or at least once per month until the disease, condition, or disorder is substantially treated or controlled.

In yet another aspect, the composition is administered via sustained drug release to the subject for a period of at least one month, at least two months, at least three months, or at least six months.

In still another aspect, an intravitreal injection or an injection into, or near, a diseased tissue is administered to the subject according to a regimen recommended by a medical practitioner for a particular patient. For example, an injection may be administered at least once per month, at least once every two months, at least once every three months, at least once every four months or at least once every six months until the disease, condition, or disorder is substantially treated or controlled. In one embodiment, treatment may be administered more frequently at the beginning, and then less frequently after a period of time. Such period of time may be determined by a medical practitioner. The concentration of an antibody fusion protein of the present invention in such an ophthalmic composition can be in the range from about 0.1 to about 200 mg/ml (or, alternatively, from about 0.25 to about 200 mg/ml, or from about 0.25 to about 160 mg/ml, or from about 0.5 to about 100 mg/ml, or from about 0.25 to about 80 mg/ml, or from about 0.5 to about 200 mg/ml, or from about 0.5 to about 160 mg/ml, or from about 0.5 to about 100 mg/ml, or from about 0.5 to about 80 mg/ml, or from about 1 to about 200 mg/ml, or from 1 to about 160 mg/ml, or from about 0.5 to about 100 mg/ml, or from about 1 to about 80 mg/ml).

In still another aspect, a method for preparing a composition of the present invention comprises combining: (a) an amount of a trispecific antibody fusion protein of the present invention; and (b) a physiologically acceptable carrier.

In one embodiment, such a physiologically acceptable carrier can be a sterile saline solution or a physiologically acceptable buffer. In another embodiment, such a carrier comprises a hydrophobic medium, such as a pharmaceutically acceptable oil. In still another embodiment, such a carrier comprises an emulsion of a hydrophobic material and water. In yet another embodiment, an antibody fusion protein of the present invention may be associated or linked with a high-molecular weight material to provide a long circulation time.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution. Non-limiting examples of buffers used for injectable compositions comprising biologics include phosphate, citric acid, acetic acid, tromethamine, histidine, arginine, gluconic acid, lactic acid, tartaric acid, aspartic acid, and glutamic acid.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of 6.8-8.2; BES (N,N-bis {2-hydroxyethyl}2-aminoethanesulfonic acid) having pKₐ of 7.1 at 25° C. and pH in the range of 6.4-7.8; MOPS (3-{N-morpholino}propanesulfonic acid) having pKₐ of 7.2 at 25° C. and pH in the range from of 6.5-7.9; TES (N-tris {hydroxymethyl}-methyl-2-aminoethanesulfonic acid) having pKₐ of 7.4 at 25° C. and pH in the range of 6.8-8.2; MOBS (4-{N-morpholino}butanesulfonic acid) having pKₐ of 7.6 at 25° C. and pH in the range of 6.9-8.3; DIPSO (3-(N,N-bis {2-hydroxyethyl}amino)-2-hydroxypropane)) having pKₐ of 7.52 at 25° C. and pH in the range of 7-8.2; TAPSO (2-hydroxy-3 {tris(hydroxymethyl)methylamino}-1-propanesulfonic acid)) having pKₐ of 7.61 at 25° C. and pH in the range of 7-8.2.

In certain embodiments, a composition of the present invention is formulated in a buffer having an acidic pH, such as from about 4 to about 6.8, or alternatively, from about 5 to about 6.8. In such embodiments, the buffer capacity of the composition desirably allows the composition to come rapidly to a physiological pH after being administered into the patient.

In addition to a buffer, a composition of the present invention can comprise a material selected from the group consisting of surfactants, stabilizers, preservatives, co-solvent, humectants, emollients, chelating agents, tonicity-adjusting agents, and antioxidants.

In one aspect, any of these materials that may be used in a composition of the present invention is a physiologically acceptable material. In certain embodiments, any of these materials that may be used in a composition of the present invention is an ophthalmically or systemically acceptable material.

Water-soluble preservatives that may be employed include quaternary ammonium compounds such as benzalkonium chloride and various polyquaternium compounds. These agents may be present in individual amounts of from about 0.001 to about 2% by weight (preferably, about 0.01% to about 0.05% by weight).

Non-limiting examples of surfactants include, but are not limited to, non-ionic surfactants, for example, polysorbates (such as polysorbate 20, polysorbate 80), 4-(1,1,3,3-tetramethylbutyl) phenol/poly(oxyethylene) polymers (such as the polymer sold under the trademark Tyloxapol), poly(oxyethylene)-poly(oxypropylene) block copolymers, glycolic esters of fatty acids and the like, and mixtures thereof.

In one aspect, the pH of the composition is in the range from about 4 to about 8. Alternatively, the pH of the composition is in the range from about 6 to about 8, or from about 6.5 to about 8, or from about 6.5 to about 7.5.

In another aspect, the composition has a pH of about 7. Alternatively, the composition has a pH in a range from about 7 to about 7.5.

In still another aspect, the composition has a pH of about 7.4.

In yet another aspect, a composition also can comprise a viscosity-modifying compound designed to facilitate the administration of the composition into the subject or to promote the bioavailability in the subject. In still another aspect, the viscosity-modifying compound may be chosen so that the composition is not readily dispersed after being administered into an environment of an eye. Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cps”) or mPa·s.

Non-limiting examples of chelating agents include ethylenediaminetetraacetic acid (“EDTA”), diethylenetriaminepentakis(methylphosphonic acid), etidronic acid, tetrasodium salt of etidronic acid (also known as “HAP”).

While the buffer itself is a “tonicity-adjusting agent” and a “pH-adjusting agent” that broadly maintains the ophthalmic solution at a particular ion concentration and pH, additional “tonicity-adjusting agents” can be added to adjust the final tonicity of the solution. Such tonicity-adjusting agents are well known to those of skill in the art and include, but are not limited to, mannitol, sorbitol, dextrose, sucrose, urea, propylene glycol, and glycerin. Also, various salts, including halide salts of a monovalent cation (e.g., NaCl or KCl) can be utilized. Typically, the tonicity of a formulation of the present invention is in the range from about 200 to 400 mOsm/kg. Alternatively, the tonicity of a formulation of the present invention is in the range from about 220 to 400 mOsm/kg, or from about 220 to 350 mOsm/kg, or from about 220 to 300 mOsm/kg, or from about 250 to 350 mOsm/kg.

Non-limiting examples of anti-oxidants include ascorbic acid (vitamin C) and its salts and esters; tocopherols (such as α-tocopherol) and tocotrienols (vitamin E), and their salts and esters (such as vitamin E TGPS (D-α-tocopheryl polyethylene glycol 1000 succinate)); glutathione; lipoic acid; uric acid; butylated hydroxyanisole (“BHA”); butylated hydroxytoluene (“BHT”); tertiary butylhydroquinone (“TBHQ”); and polyphenolic anti-oxidants (such as gallic acid, cinnanmic acid, flavonoids, and their salts, esters, and derivatives).

Non-limiting examples of stabilizers includes sucrose, mannitol, sorbitol, and trehalose.

It should be understood that the proportions of the various components or mixtures may be adjusted for the appropriate circumstances.

In another aspect, an antibody fusion protein of the present invention and appropriate amounts of one or more desired excipients are incorporated into a formulation for topical administration or injection to a portion of the eye, such as the anterior or posterior segment, or the vitreous humor. An injectable formulation can desirably comprise a carrier that provides a sustained-release of the active ingredients, such as for a period longer than about 1 week (or longer than about 1, 2, 3, 4, 5, or 6 months). In certain embodiments, an antibody fusion protein of the present invention is included in a delivery device for sustained release of the active ingredients over a long period of time, such as 4, 5, 6 months or longer. An example of such delivery device is described in U.S. Pat. Nos. 8,399,006 and 9,417,238.

In still another aspect, a composition comprising an antibody fusion protein of the present invention and desired excipients is lyophilized and is reconstituted with a physiologically acceptable liquid carrier substantially immediately before administration to a subject.

In one embodiment, a compound or composition of the present invention can be injected with a fine-gauge needle, such as 25-35 gauge. Typically, an amount from about 25 μl to about 100 μl of a composition comprising about 25-4000 μg of an antibody fusion protein of the present invention is administered into a patient. In one aspect, the antibody fusion protein has an amino acid sequence selected from the group consisting of SEQ ID NOs: 12-29, 38-41, and 57-62, and conservatively modified variants thereof. A concentration of such antibody fusion protein is selected from the ranges disclosed above. Other antibody fusion proteins comprising various Ang-2-, VEGF-, and IL-6R-binding units as disclosed herein can also be incorporated into compositions disclosed herein.

In still another aspect, an antibody fusion protein of the present invention is incorporated into an ophthalmic device that comprises a biodegradable material, and the device is implanted into a posterior-segment tissue of a subject to provide a long-term (e.g., longer than about 1 week, or longer than about 1, 2, 3, 4, 5, or 6 months) treatment or control of an angiogenic disease, condition, or disorder. Such a device may be implanted by a skilled physician in the subject's ocular or periocular tissue. Non-limiting examples of ophthalmic implant systems or devices for the sustained-release of an active ingredient are disclosed in U.S. Pat. Nos. 5,378,475; 5,773,019; 5,902,598; 6,001,386; 6,051,576; and 6,726,918.

In still another aspect, a method for treating or controlling an ophthalmic angiogenic disease, condition, or disorder comprises administering a composition comprising an antibody fusion protein of the present invention to a subject in need thereof.

In still another aspect, a method for treating or controlling an ophthalmic angiogenic disease, condition, or disorder comprises administering a composition comprising an antibody fusion protein having an amino acid sequence selected from the group consisting of SEQ ID NOs: 12-29, 38-41, and 57-62, and conservatively modified variants thereof to a subject in need of such treatment or control. Other antibody fusion proteins comprising various Ang-2-, VEGF-, and IL-6R-binding units as disclosed herein can also be used in such method.

In still another aspect, a method for treating or controlling an ophthalmic angiogenic disease, condition, or disorder having an etiology in aberrant angiogenesis of in the posterior segment of an eye comprises intravitreally injecting a composition comprising an antibody fusion protein having an amino acid sequence selected from the group consisting of SEQ ID NOs: 12-29, 38-41, and 57-62, and conservatively modified variants thereof.

In another embodiment, such disease, condition, or disorder is selected from the group consisting of: macular edema resulting from diabetes, uveitis, central and branch retinal vein occlusion, choroidal neovascularization including neovascular age-related macular degeneration (wet age-related macular degeneration), polypoidal choroidal vasculopathy (PCV) and myopic choroidal neovacular degeneration, vascular leak, non-proliferative and proliferative diabetic retinopathy, retinopathy of prematurity, corneal neovascularization, corneal inflammation, and neovascular glaucoma.

In yet another aspect, a composition of the present invention is administered once a week, once a month, once a year, twice a year, three times a year, four times a year, or at a suitable frequency that is determined to be appropriate for treating or controlling an anterior-segment inflammatory disease, condition, or disorder.

In still another aspect, an antibody fusion protein of the present invention can also be used for the treatment or control of tumors, systemic inflammatory diseases or conditions, or autoimmune diseases such as arthritis. Such treatment or control may be effected by, for example, systemic administration. Dosages and regimens for treating such diseases or conditions may be determined or recommended for the particular disease or condition by medical practitioners.

### Example 1: Expression and Purification of Fusion Proteins of the Present Invention

Fusion proteins of the present invention were successfully expressed in Chinese hamster ovarian (“CHO”) cells. With one round of purification by affinity chromatography, purity of >95% was achieved for most proteins produced.

CDNAs encoding the amino acid sequences of fusion proteins identified in Table 3 were synthetized, and an expressing vector based on the circular pcDNA3.4 vector system was constructed for each cDNA. The expression vector was used to transiently transfect CHO cells with chemically defined culture media. The produced protein was purified by Protein-A-affinity-column ultrafiltration and then subjected to 0.2 μm sterile filtration to get the bulk of high purity. Purity ranged from 82.9% to 100% after one round purification by affinity chromatography and analyzed by size exclusion chromatography (SEC-HPLC).

TABLE 3
Protein Yield and Purity

| | | | Purity | Purity |
|---|---|---|---|---|
| | | Total | (SEC | (SEC |
| | | Protein | 214 nm) | 280 nm) |
| Molecule ID | Alternate ID | (mg) | (%) | (%) |
| EB-105BI-7CA | B21138001 | 4.5 | 98.8 | 98.8 |
| EB-105BI-7CB | B21138002 | 5.0 | 95.6 | 95.8 |
| EB-105BI-10C | B21138003 | 16.9 | 94.1 | 94.4 |
| EB-105BI- | B21138004 | 8.5 | 97.3 | 97.4 |
| 15CA | | | | |
| EB-105BI- | B21138005 | 0.8 | 96.2 | 96.3 |
| 15CB | | | | |
| EB-105BI-21C | B21138006 | 8.7 | 84.1 | 84.6 |
| EB-105BI- | B21138007 | 7.8 | 82.9 | 83.3 |
| Con4C | | | | |
| EB-105BI- | B21138008 | 14.9 | 100 | 100 |
| Con4-40CA | | | | |
| EB-105BI- | B21138009 | 15.0 | 96.7 | 96.8 |
| Con4-40CB | | | | |
| EB-105BIc1 | MFDDHD001 | 15.75 | 100 | 100 |
| EB-105BIc2 | MFDDHD002 | 25.87 | 95.9 | 96.6 |
| EB-105BId1 | MFDDHD003 | 3.32 | 95.1 | 95.9 |
| EB-105BId2 | MFDDHD004 | 1.90 | 100 | 100 |
| EB-105BId3 | MFDDHD005 | 24.25 | 89.97 | 90.4 |
| EB-105BId4 | MFDDHD006 | 9.03 | 99.5 | 99.6 |
| EB-105BIel | MFDDHD007 | 22.14 | 95.1 | 95.5 |
| EB-105BIe2 | MFDDHD008 | 12.80 | 98.5 | 98.5 |

### Example 2: ELISA Binding Affinities for Human VEGF-A165

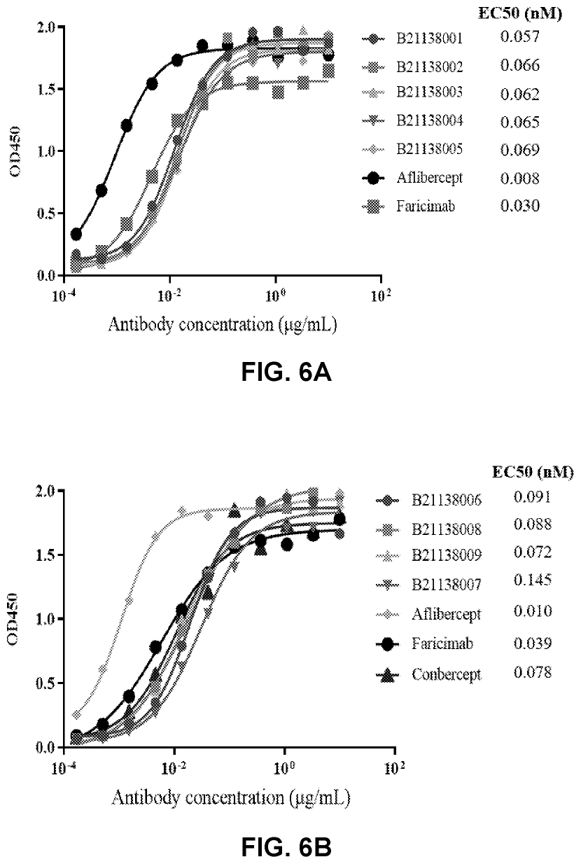

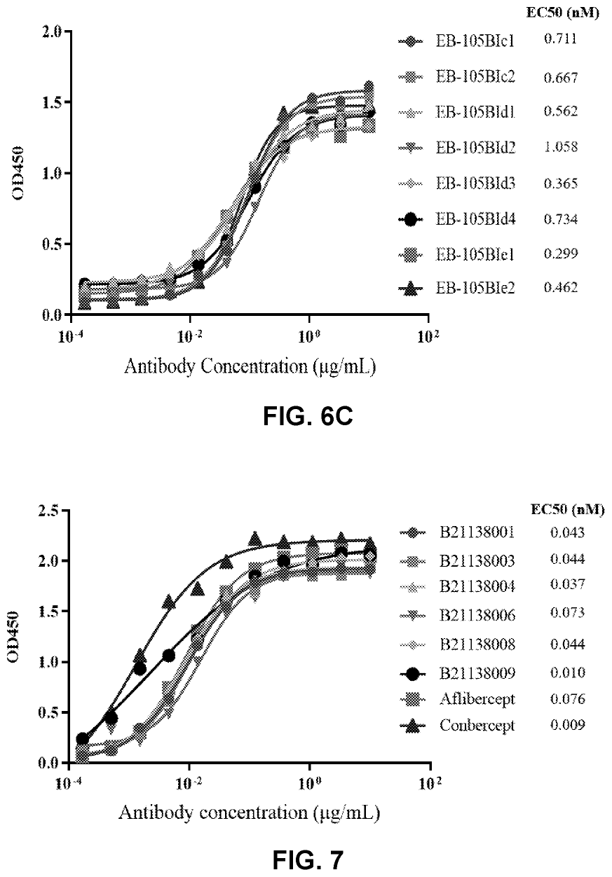

ELISA assay was performed using 96-well plates coated with recombinant human VEGF-A165 (4 μg/ml, 50 μl/well) at +4° C. for 16 hours. After non-specific blocking using 1% BSA at 25° C. for 1 hour, a series of dilutions of test antibodies (“Abs”) were added into the coated wells and incubated at 25° C. for 1 hour. The bound Abs were detected using a secondary Ab (goat anti-human IgG1-Fc) conjugated with HRP followed by OD₄₅₀ reading. The binding affinities of EB-105 series of molecules for human VEGF-A165 are at sub-nanomolar scales and comparable to aflibercept, faricimab and conbercept. See FIGS. 6A-6C. Aflibercept and conbercept are two human Fc fusion proteins that bind to VEGF-A, B and PIGF. Faricimab is a bi-specific antibody that binds to VEGF-A and Ang-2.

### Example 3: ELISA Binding Affinities of B21138001-B21138009 for Human VEGF-B167

The same ELISA procedure was performed with recombinant human VEGF-B167. The binding affinities of EB-105 series of molecules for human VEGF-B167 are at sub-nanomolar scales and comparable to aflibercept and conbercept. See FIG. 7.

### Example 4: ELISA Binding Affinities of B21138001-B21138009 for Human PIGF

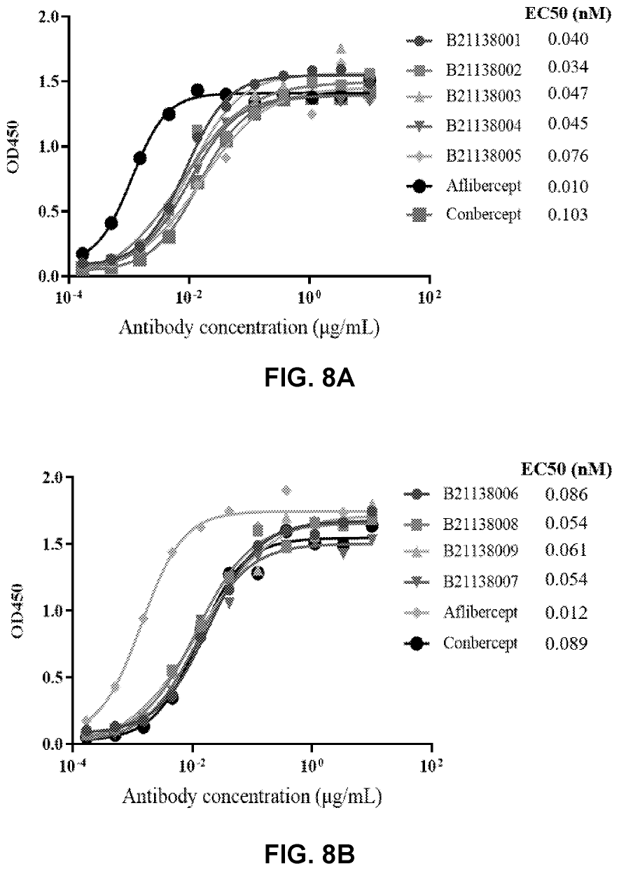

The same ELISA procedure was performed with recombinant human PIGF. The binding affinities of EB-105 series of molecules for human PIGF are at sub-nanomolar scales and comparable to aflibercept and conbercept. See FIG. 8A and FIG. 8B.

### Example 5: ELISA Binding Affinities for Human IL-6R

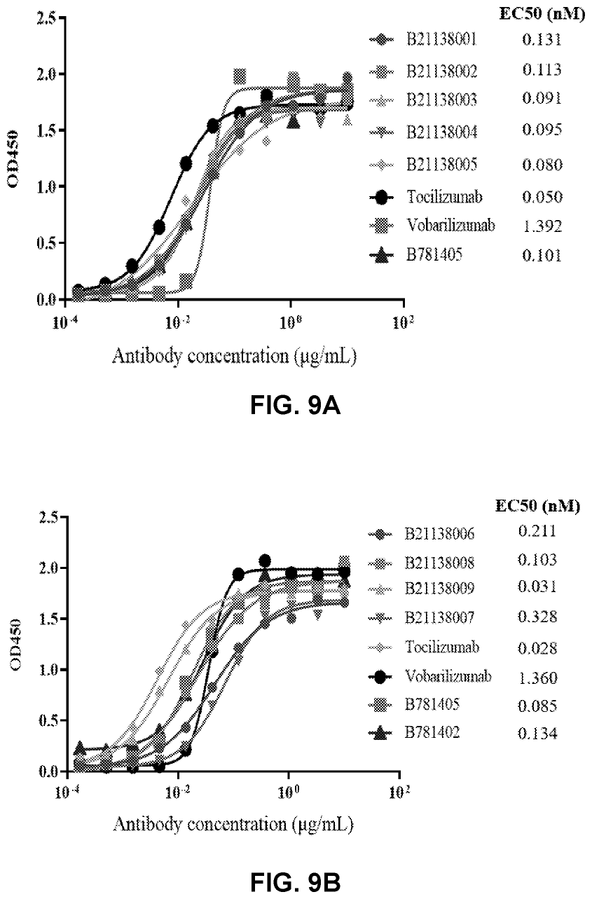

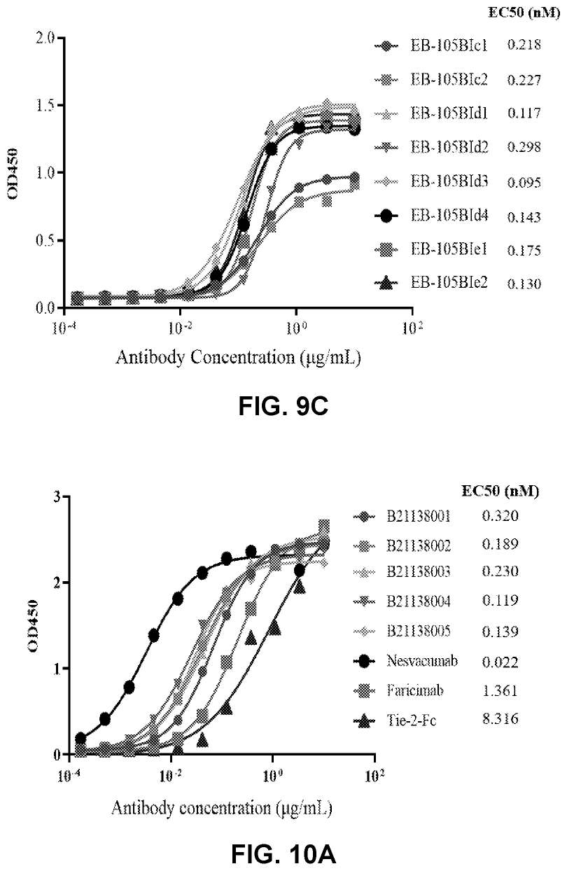

The same ELISA procedure was performed with recombinant human IL-6R. The binding affinities of EB-105 series of molecules for human IL-6R are at sub-nanomolar scales and comparable to tocilizumab and vobarilizumab. Tocilizumab is a clinical stage mAb against IL-6R. Vobarilizumab is a single domain antibody against IL-6R. B781402 and B781405 are bispecific fusion antibodies that inhibit VEGF-A, B, PIGF and IL-6R. See FIGS. 9A-9C.

### Example 6: ELISA Binding Affinities for Human Ang-2

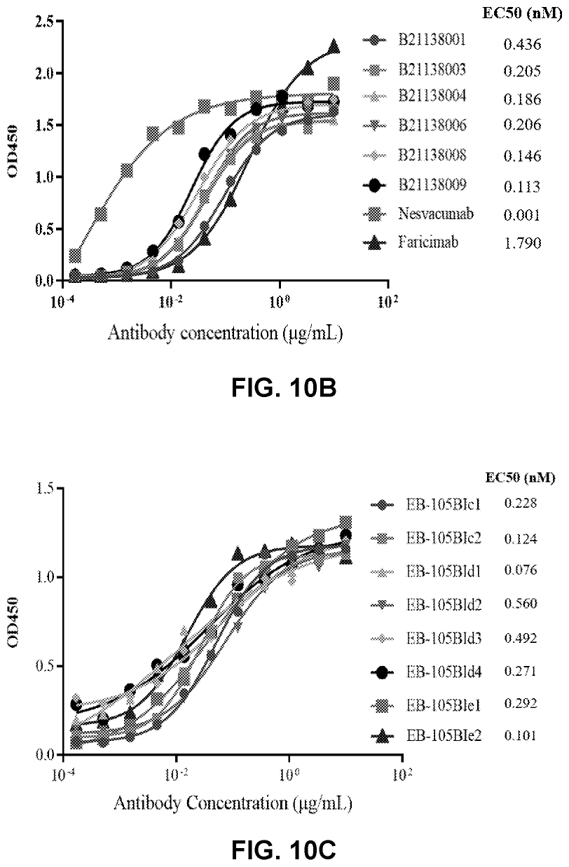

The same ELISA procedure was performed with recombinant human Ang-2. The binding affinities of EB-105 series of molecules for human Ang-2 are at sub-nanomolar scales and comparable to nesvacumab and faricimab. Nevascumab is an mAb against human Ang-2. Faricimab is a clinical-stage bispecific antibody for inhibition of VEGF-A and Ang-2. See FIGS. 10A-10C.

### Example 7: ELISA Binding Affinities of B21138001, B21138003, B21138004, B21138006, B21138008, and B21138009 for Human Ang-1

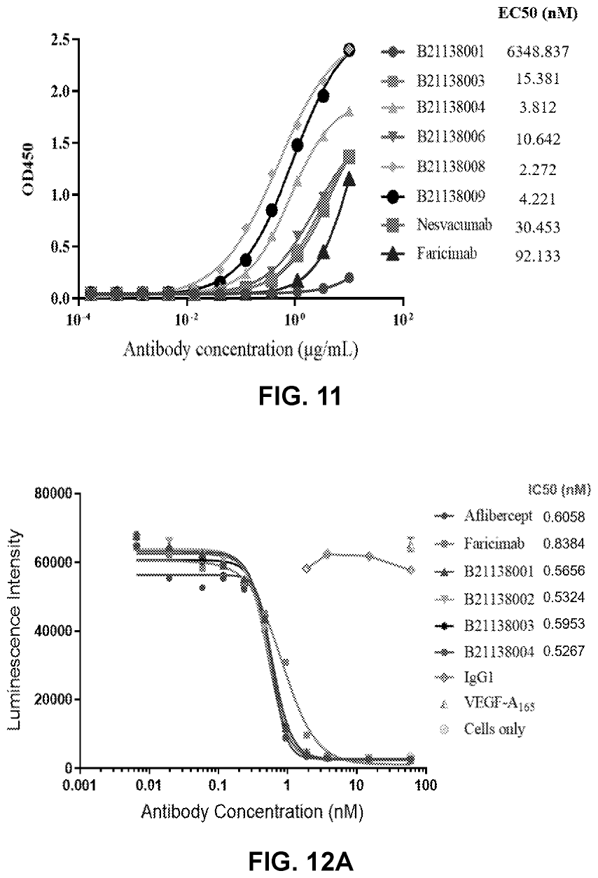

The same ELISA procedure was performed with recombinant human Ang-1. B21138001 showed very low binding affinities for human Ang-1. Affinities of B21138003 and B21138006 for human Ang-1 are comparable to that of nesvacumab but somewhat higher than that of faricimab. See FIG. 11.

### Example 8: SPR Biacore Binding Affinity Assays

SPR Biacore assay was performed at 25° C. with HBS-EP+ as a running buffer. The assay was performed by immobilization of the anti-human IgG (Fc) antibody onto the CM5 sensor chip surface and determined the level of ligand immobilization. The amount of anti-Fc antibody coupling onto the CM5 sensor chip was about 7,000~14,000 response unit (RU). Test antibodies were injected over the surface of Series S Sensor Chip CM5 as Capture. The analytes (target proteins) were diluted with running buffer into different concentrations and injected over the sensor surface as the association phase for affinity and kinetics measurements. The 1:1 binding model was used to measure the binding affinity and/or kinetics.

SPR Biacore assays indicate that EB-105 molecules bind to target proteins at picomolar or sub-nanomolar scales (Tables 4 and 5). B21138001 and B21138002 potently bind to human VEGF-A165, VEGF-B167 and PIGF, with binding affinities comparable to aflibercept. They also bind to human IL-6R and Ang-2, with binding affinities comparable to tocilizumab and faricimab (Tables 4 and 5). Tocilizumab is a clinical stage mAb against human IL-6R. Faricimab is the first FDA approved bispecific antibody that binds to VEGF-A and Ang-2 for the treatment of DME.

TABLE 4
Binding Affinities of B21138001 and Its Comparators for Target Proteins Measured by
SPR Biacore Assays

| | | Chi² | kₐ | k_{d} | K_{D} | Rₘₐₓ |
|---|---|---|---|---|---|---|
| Ligand | Analyte | (RU²) | (1/Ms) | (1/s) | (M) | (RU) |
| B21138001 | VEGF-A₁₆₅ | 5.54E−02 | 3.22E+07 | 4.24E−04 | 1.32E−11 | 30.2 |
| Aflibercept | | 1.06E−01 | 2.17E+07 | 5.59E−05 | 2.57E−12 | 42.2 |
| B21138001 | VEGF-B₁₆₇ | 1.64E+00 | 4.21E+05 | 4.41E−04 | 1.05E−09 | 73.3 |
| Aflibercept | | 9.36E−01 | 4.61E+05 | 8.54E−04 | 1.85E−09 | 52.2 |
| B21138001 | PlGF | 7.03E−02 | 2.00E+07 | 8.41E−03 | 4.20E−10 | 22.5 |
| Aflibercept | | 1.02E−01 | 6.13E+06 | 9.08E−04 | 1.48E−10 | 47.6 |
| B21138001 | IL-6R | 2.87E−01 | 1.41E+06 | 7.76E−04 | 5.51E−10 | 66.5 |
| Tocilizumab | | 2.01E−01 | 4.20E+05 | 4.60E−04 | 1.10E−09 | 56.1 |
| B21138001 | Angiopoietin-2 | 1.36E−01 | 1.19E+06 | 3.66E−04 | 3.07E−10 | 36 |
| Faricimab | | 3.90E−01 | 8.03E+05 | 9.94E−05 | 1.24E−10 | 80.4 |
| B21138001 | Angiopoietin 1 | | | | No binding | |

Chi² (RU²): Goodness of fit reflected by resonance units (RU).
kₐ: kinetic binding associate rate.
k_{d}: kinetic binding dissociate rate.
K_{D}: equilibrium binding constant calculated by k_{d}/kₐ.
Rₘₐₓ (RU): Max response for analyte binding.

TABLE 5
Binding Affinities of B21138002 and Its Comparators for Target Proteins Measured by
SPR Biacore Assays

| | | Chi² | kₐ | k_{d} | K_{D} | Rₘₐₓ |
|---|---|---|---|---|---|---|
| Ligand | Analyte | (RU²) | (1/Ms) | (1/s) | (M) | (RU) |
| B21138002 | VEGF-A₁₆₅ | 5.72E−02 | 2.71E+07 | 6.79E−04 | 2.50E−11 | 60.9 |
| Aflibercept | | 1.54E−01 | 1.74E+07 | 6.00E−05 | 3.44E−12 | 63.2 |
| B21138002 | VEGF-B₁₆₇ | 2.33E+00 | 5.78E+05 | 5.42E−04 | 9.38E−10 | 74.9 |
| Aflibercept | | 1.10E+00 | 5.62E+05 | 7.72E−04 | 1.37E−09 | 51.0 |
| B21138002 | PlGF | 2.22E−01 | 1.58E+07 | 6.31E−03 | 4.00E−10 | 62.4 |
| Aflibercept | | 7.59E−02 | 6.21E+06 | 6.61E−04 | 1.06E−10 | 48.2 |
| B21138002 | IL-6R | 1.55E−01 | 1.47E+06 | 6.98E−04 | 4.74E−10 | 69.8 |
| Tocilizumab | | 2.87E−02 | 3.73E+05 | 5.21E−04 | 1.40E−09 | 99.2 |
| B21138002 | Angiopoietin-2 | 2.54E−01 | 8.78E+05 | 2.84E−04 | 3.23E−10 | 43.1 |
| Faricimab | | 3.11E−01 | 7.42E+05 | 1.37E−04 | 1.84E−10 | 66.8 |
| B21138002 | Angiopoietin 1 | | | | No binding | |

Chi² (RU²): Goodness of fit reflected by resonance units (RU).
kₐ: kinetic binding associate rate.
k_{d}: kinetic binding dissociate rate.
K_{D}: equilibrium binding constant calculated by kd/ka.
Rₘₐₓ (RU): Max response for analyte binding.

### Evaluation of In Vitro Cell-Based Functions of Antibody Fusion Proteins of the Present Invention

### Example 9: Inhibition of VEGF-A165-Mediated VEGFR-2 Signaling

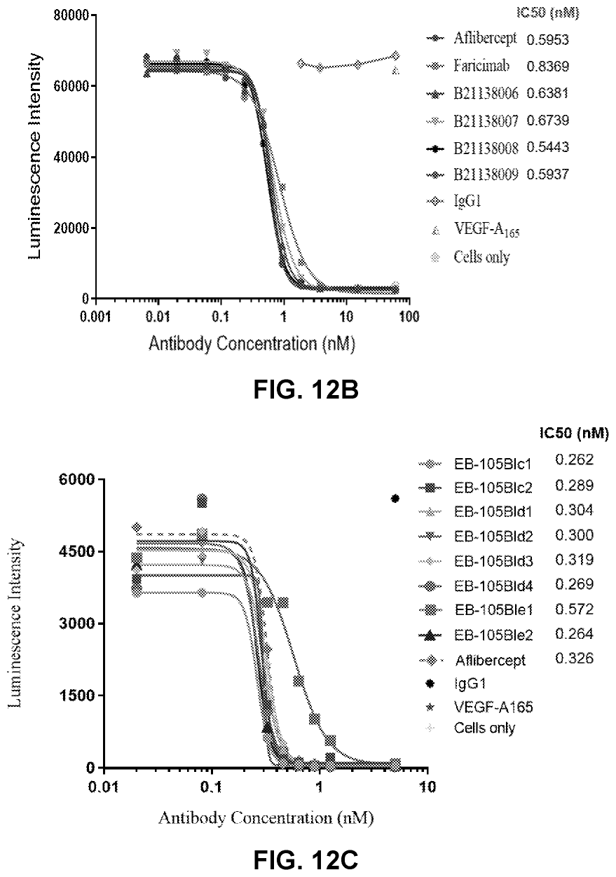

The effect of EB-105 molecules on inhibiting VEGF-A165-mediated VEGFR-2 signaling was studied using an engineered HEK-293 cell line, in which the firefly luciferase gene is expressed under the control of nuclear factor activated T cell response elements (VEGFR-2-NF-AT). Aflibercept and faricimab were used as two comparators. The results indicate that EB-105 molecules are comparable to aflibercept and faricimab in dose-dependent inhibition of VEGF-A165-stimulated VEGFR-2 signaling. See FIGS. 12A-12C.

Serial dilutions of EB-105 molecules, aflibercept and faricimab were incubated with 60 ng/ml of human VEGF-A165 at room temperature for 30 minutes, then VEGFR-2 luciferase reporter cells were added into each well to study the effects of test materials on inhibiting VEGF-A165 mediated VEGFR-2 signaling. EB-105 molecules are comparable to aflibercept and faricimab in inhibiting VEGF-A165-stimulated VEGFR-2 signaling in a dose response manner as indicated by comparable IC50 values. Aflibercept is a human Fc fusion protein containing VEGFR-1-D2 and VEGFR-2-D3. Faricimab is a bispecific antibody that binds to VEGF-A and Ang-2.

### Example 10: Inhibition of Ang-2/Tie-2 Interaction

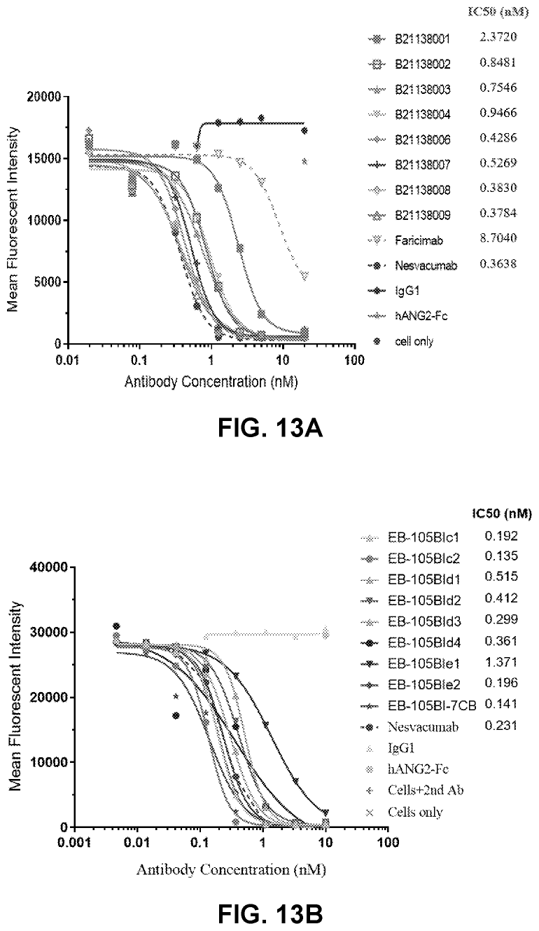

The effect of EB-105 molecules on inhibiting Ang-2/Tie-2 interaction was studied by fluorescence-activated cell sorting (FACS) analysis using engineered HEK-293 cells that overexpress human Tie-2 receptor, with faricimab and nesvacumab as two comparators. Nesvascumab is an mAb against Ang-2. Serial dilutions of EB-105 molecules, faricimab, and nesvascumab were incubated with 100 ng/ml of human Ang-2 at 4° C. for 60 minutes, then Tie-2-expressing cells were added into each well to study the effects of test antibodies on blocking Ang-2 binding to Tie-2 receptor via FACS analysis. Our results indicate that EB-105 molecules, in a dose response manner, showed a similar effect to nesvacumab and were more potent than faricimab in blocking Ang-2 binding to Tie-2 receptor. See FIG. 13A and FIG. 13B.

### Example 11: Inhibition of Ang-1/Tie-2 Interaction

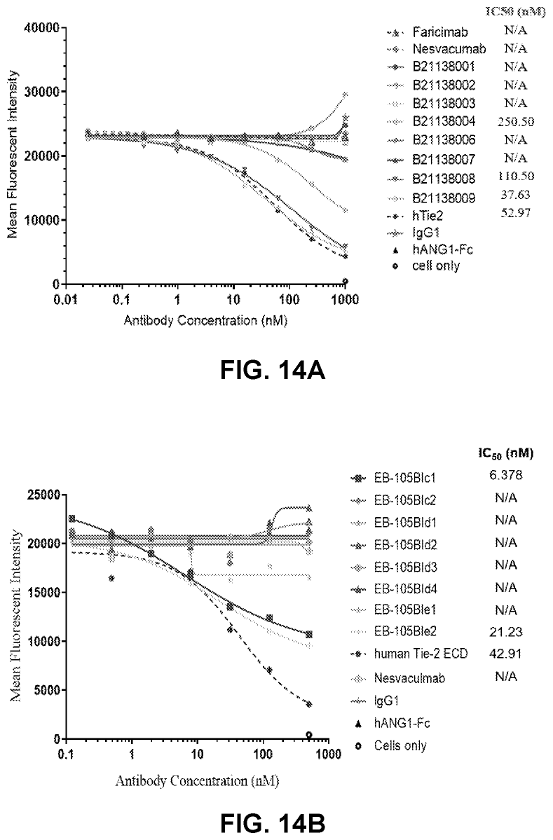

The effect of EB-105 molecules on Ang-1/Tie-2 interaction was studied by FACS analysis using engineered HEK-293 cells that overexpress human Tie-2 (hTie-2) receptor, with faricimab and nesvascumab as two comparators. Serial dilutions of EB-105, faricimab, nesvascumab, and hTie-2 were incubated with 2 μg/ml of human Ang-1-Fc at 4° C. for 60 minutes, then Tie-2-expressing cells were added into each well to study the effects of test materials on blocking Ang-1 binding to Tie-2 receptor. Recombinant hTie-2 was used as a positive control. Similar to faricimab and nesvascumab, most EB-105 molecules including B21138001, B21138002, B21138003, B21138006 and B21138007, do not affect the binding of Ang-1 to Tie-2 receptor. B21138004, B21138008 and B21138009 showed some effects on inhibiting Ang-1 binding to Tie-2 receptor. See FIG. 14A and FIG. 14B.

### Example 12: Inhibition of IL-6 Binding to IL-6R

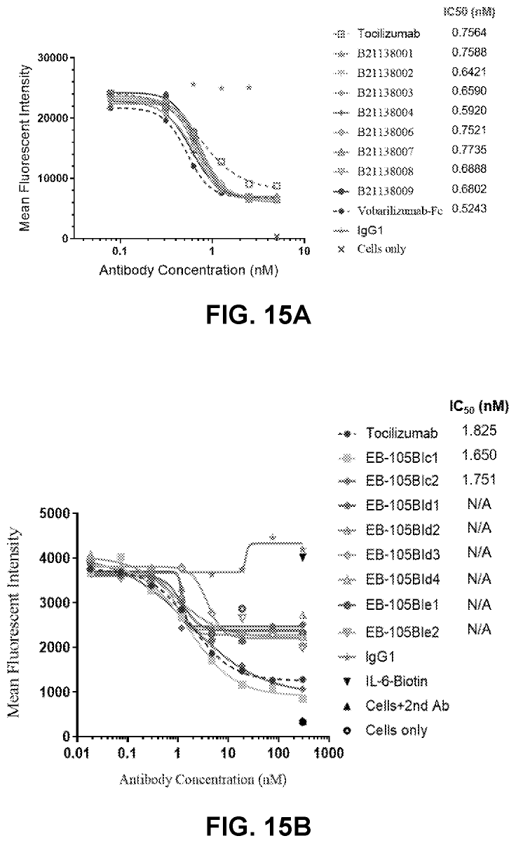

The effect of EB-105 molecules on blocking IL-6 binding to IL-6R was studied by FACS analysis using engineered CHO—S cells which overexpress human IL-6R, with tocilizumab and vobarilizumab as two comparators. Vobarilizumab is a single domain antibody (also termed nanobody) against human IL-6R. Serial dilutions of EB-105 molecules, tocilizumab, and vobarilizumab were incubated with biotinylated human IL-6 at 4° C. for 60 minutes, then IL-6R-expressing CHO—S cells were added into each well to study the effects of test materials on blocking biotinylated IL-6 binding to IL-6R. Our results indicate that EB-105 molecules are comparable to tocilizumab and vobarilizumab in blocking IL-6 binding to IL-6R in a dose response manner. See FIG. 15A and FIG. 15B.

### Example 13: Cross Species Activities of Lead Molecule of EB-105 (B21138002) for Target Proteins in Humans, Monkeys, Rabbits, and Rats

Enzyme-linked immunosorbent assay (ELISA) was performed to assess the cross species binding affinities of the lead molecule B21138002 for VEGF-A, IL-6R, and Ang-2 in humans, Rhesus monkeys, rabbits, and rats. B21138002 potently binds to human, Rhesus monkey, and rabbit VEGF-A165, Ang-2, and IL-6R at sub-nanomolar scales. B21138002 binds to rat VEGF-A165 and Ang-2 but not IL-6R. See Table 6.

TABLE 6

| Cross Species Binding Affinities of B21138002 | | | |
|---|---|---|---|
| for Target Proteins Measured by ELISA | | | |
| Species | VEGF-A165 (nM) | Ang-2 (nM) | IL-6R (nM) |
| Human | 0.0621 | 0.0643 | 0.0359 |
| Monkey | 0.0621 | 0.0729 | 0.0498 |
| Rabbit | 0.0775 | 0.1726 | 0.0630 |
| Rat | 0.0207 | 0.5077 | No binding |

### Pharmacology Studies in Two Animal Models

B21138002, a lead antibody fusion protein of the present invention, was formulated in a phosphate buffer solution for intravitreal (IVT) injection to study its efficacy and exploratory safety in two animal models: pre-retinal neovascularization (PRN) in Dutch belted rabbits and laser-induced choroidal neovascularization (CNV) in cynomolgus monkeys as described below.

### Example 14: Effect of a Single Dose IVT Injection of B21138002 on Vascular Leakage from PRN in Rabbits

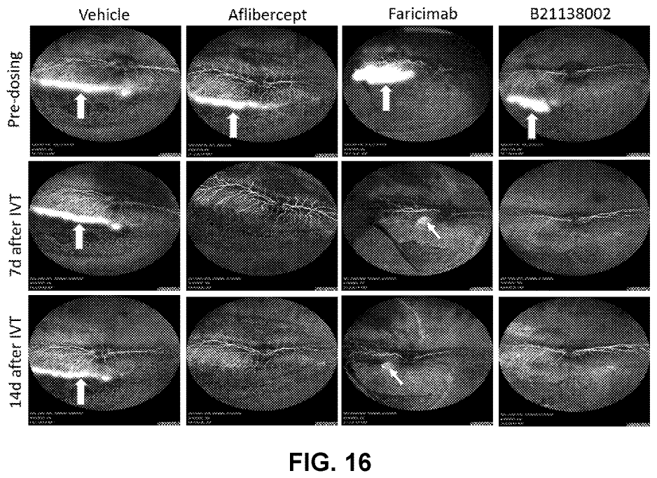

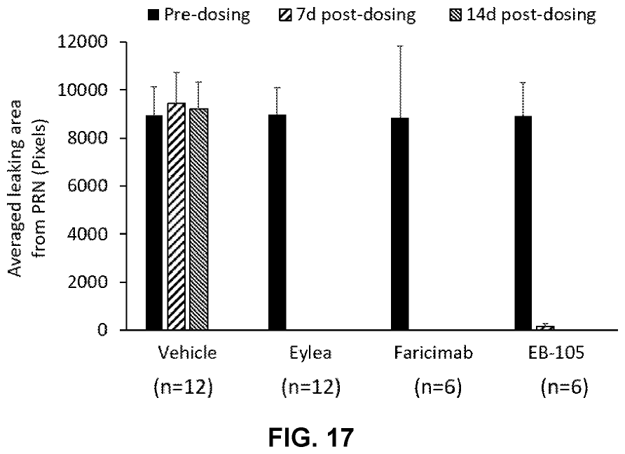

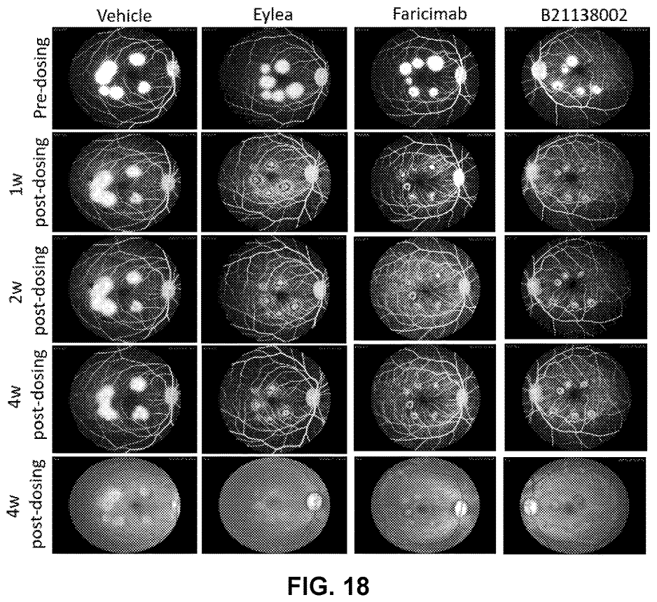

PRN was induced by IVT injection of DL-α-aminoadipic acid (DL-AAA, 80 mM, 50 μl/eye) in Dutch belted rabbits. After confirmation of the development of PRN eight weeks post DL-AAA injection (FIG. 15, pointed by arrows in the top/first row of the composite fundus fluorescein angiography images), each eye received a single IVT injection of 50 μl of vehicle, equivalent molar amounts of aflibercept (EYLEA®, 0.28 mg/eye), faricimab (VABYSMOR, 0.36 mg/eye), or B21138002 (0.5 mg/eye) to study their effects on inhibiting vascular leakage from existing PRN. Fundus fluorescein angiography (FFA) was performed to monitor changes of vascular leakage overtime. As demonstrated, B21138002 effectively inhibited vascular leakage, with an apparent efficacy comparable to aflibercept and faricimab (FIGS. 16 and 17). Aflibercept and faricimab are FDA-approved treatment for DME.

Persistent vascular leakage in vehicle treated group before, 7 and 14 days after IVT injection (n=12). Eyes receiving aflibercept (n=12), faricimab (n=6), or B21138002 (n=6) showed highly effective inhibition of vascular leakage 7 and 14 days after treatment. The larger arrows in FIG. 16 point to extensive leakage from well-developed PRN, while the smaller arrows point to residual fluorescein leakage from one area of an eye treated by faricimab. FIG. 17 presents the results of the quantitative analysis of vascular leakage area seven and fourteen days after IVT injections of the vehicle and test materials. Aflibercept, faricimab, and B21138002 effectively inhibited vascular leakage seven and fourteen days after IVT injection, as compared to pre-dosing leakage, at p value <0.01.

### Example 15: Effect of a Single-Dose IVT Injection of B21138002 on Vascular Leakage in Laser-Induced Choroidal Neovascularization (CNV) in Monkeys

The effect of B21138002 on inhibition of vascular leakage was studied in laser-induced CNV in cynomolgus monkeys, with aflibercept and faricimab as two clinical comparators. The development of CNV was confirmed by FFA after twelve days of intensive laser photocoagulation. Two days later (fourteen days after laser photocoagulation), each eye received a single IVT injection of 50 μl of vehicle, 0.5 mg/eye of aflibercept, faricimab, or B21138002. Color fundus photography and FFA were performed to monitor changes in the fundus and vascular leakage 1, 2, and 4 weeks after treatment. Similar to our findings in DL-AAA induced PRN rabbit study, B21138002 effectively inhibited leakage from CNV lesions, with an apparent efficacy comparable to aflibercept and faricimab (FIGS. 18-20). Persistent vascular leakage from CNV lesions in vehicle treated group pre-dosing and 1, 2, and 4 weeks after dosing (n=6). Eyes receiving aflibercept, faricimab, or B21138002 (n=3/group) showed nearly complete inhibition of vascular leakage 1, 2 and 4 weeks after treatment. Fundus photography showed no signs of apparent retinal inflammation by 4 weeks after IVT injection of test materials, including vehicle, aflibercept, faricimab, and B21138002 (FIG. 18, last row of fundus images).

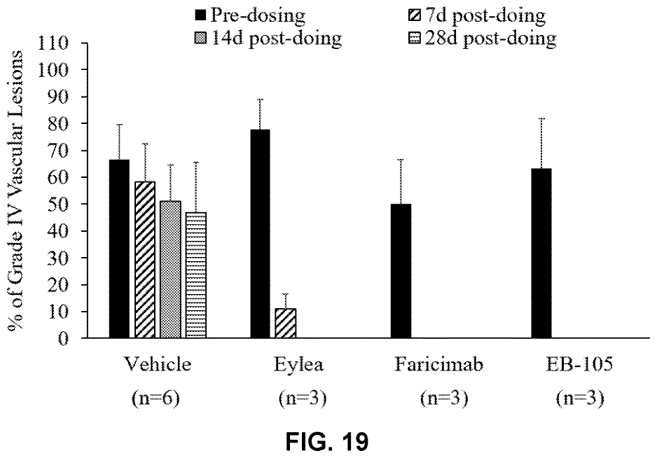

FIG. 19 shows that the percentage of grade-IV CNV lesions in vehicle treated group tended to slightly decrease with time, although the changes were not statistically significant. Aflibercept (Eylea®) dramatically reduced the number of grade-IV CNV lesions. Faricimab and B21138002 completely inhibited the development of grade-IV CNV lesions 1, 2, and 4 weeks after treatment. (Grading of CNV lesions: Grade I-no hyperfluorescence, Grade II-hyperfluorescent staining without fluorescein leakage, Grade III-early hyperfluorescence with mild fluorescein leakage confined to the border of the laser burn at late-stage of FFA, Grade IV-early hyperfluorescence with late-stage severe fluorescein leakage beyond the border of the laser burn. Grade IV lesions are considered highly clinical relevance.)

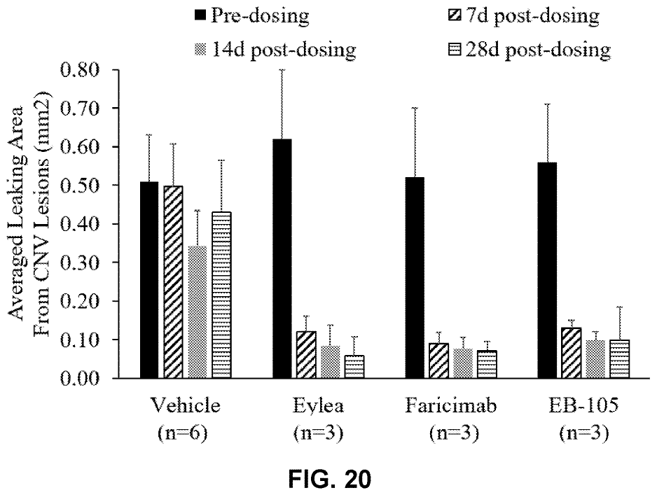

FIG. 20 shows the results of the quantitative analysis of vascular leakage in laser-induced CNV in monkeys. The vehicle treated group showed relatively persistent leakage from CNV lesions pre-dosing, and 1, 2 and 4 weeks after treatment. B21138002 dramatically inhibited CNV leakage, with an efficacy comparable to aflibercept (Eylea®) and faricimab 1, 2 and 4 weeks after treatment.

### NUCLEIC ACID AND AMINO ACID SEQUENCE LISTING

SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTII (SEQ ID
NO: 1)
DVVLSPSHGIELSVGEKLVLNCTARTELNVGIDENWEYPSSKHQHKK
LVNRDLKTQSGSEMKKFLSTLTIDGVTRSDQGLYTCAASSGLMTKKNSTFVRVH
EK (SEQ ID NO: 2)
VEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFS
CSVMHEALHNHYTQKSLSLSP (SEQ ID NO: 3)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSP (SEQ ID NO: 4)
DIQMTQSPSSLSASVGDRVTITCRASQDISSYLNWYQQKPGKAPKLLI
YYTSRLHSGVPSRFSGSGSGTDFTFTISSLQPEDIATYYCQQGNTLPYTFGQGTKV
EIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
(SEQ ID NO: 5)
AQTNFMPMDDLEQRLYEQFILQQGLE (SEQ ID NO: 6)
MGAQQKFQPLDELEQTLYEQFMLQQALE (SEQ ID NO: 7)
MGAQQKYQPLDELDKTLYDQFMLQQGLE (SEQ ID NO: 8)
MGAQHTFQPLDELEETLYYQWLYDQLLE (SEQ ID NO: 9)
AQQEECEWDPWTCEHMLE (SEQ ID NO: 10)
AQTNIQEECEWDPWTCDHMPGKLE (SEQ ID NO: 11)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSGGGGSAQTNFMPMDDLEQRLYEQF
ILQQGLE (SEQ ID NO: 12)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 13)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSAQTNFMPMDDLEQRLYEQFILQQG
LE (SEQ ID NO: 14)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 15)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSGGGGSMGAQQKFQPLDELEQTLYE
QFMLQQALE (SEQ ID NO: 16)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 17)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSGGGGSMGAQQKYQPLDELDKTLY
DQFMLQQGLE (SEQ ID NO: 18)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDFNWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 19)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSMGAQQKYQPLDELDKTLYDQFML
QQGLE (SEQ ID NO: 20)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 21)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSGGGGSMGAQHTFQPLDELEETLYY
QWLYDQLLE (SEQ ID NO: 22)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO 23)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSGGGGSAQQEECEWDPWTCEHMLE
(SEQ ID NO: 24)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 25)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSGGGGSAQTNIQEECEWDPWTCDHM
PGKLE (SEQ ID NO: 26)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 27)
QVQLQESGPGLVRPSQTLSLTCTVSGYSITSDHAWSWVRQPPGRGLE
WIGYISYSGITTYNPSLKSRVTMLRDTSKNQFSLRLSSVTAADTAVYYCARSLAR
TTAMDYWGQGSLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCS
VMHEALHNHYTQKSLSLSPGGGGSGGGGSAQTNIQEECEWDPWTCDHMPGKLE
(SEQ ID NO: 28)
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTIIDVVLSP
SHGIELSVGEKLVLNCTARTELNVGIDENWEYPSSKHQHKKLVNRDLKTQSGSE
MKKFLSTLTIDGVTRSDQGLYTCAASSGLMTKKNSTFVRVHEKDIQMTQSPSSLS
ASVGDRVTITCRASQDISSYLNWYQQKPGKAPKLLIYYTSRLHSGVPSRFSGSGSG
TDFTFTISSLQPEDIATYYCQQGNTLPYTFGQGTKVEIKRTVAAPSVFIFPPSDEQL
KSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 29)
EVQLVESGGGLVQPGGSLRLSCAASGRTFSSYDFVWVRQAPGKGLEF
VSAISVEGEHTYYADSVKGRFTISRDNAKNTLYLQMNSLRAEDTAVYYCNAVK
WSDYGPDNDYWGQGTLVTVSS (SEQ ID NO: 30)
EVQLVESGGGLVQPGGSLRLSCAASGLYFSSYDIAWVRQAPGKGLEF
VSAIDVSGEHTYYADSVKGRFTISRDNAKNTLYLQMNSLRAEDTAVYYCNAVK
WSDYGPDNDYWGQGTLVTVSS (SEQ ID NO: 31)
EVQLQESGGGLVQPGGSLRLSCAASGFTLDAYAIGWFRQAPGKEREG
VSTIGKSDGSTCYADSVKGRFTISRDNAKNTVWLQMNSLRAEDTAVYYCAARP
WWVGDAPSIAAEYEYDLWGQGTQVTVSS (SEQ ID NO: 32)
EVQLQESGGGLVQPGGSLRLSCAASGFTLDEYAIGWFRQAPGKEREG
VSCIGKADGSTCYADSVKGRFTISRDNAKNTVWLQMNSLRAEDTAVYYCAARP
WWVGDPPSIGAEYEYDLWGQGTQVTVSS (SEQ ID NO: 33)
QVQLVESGGGLVQPGGSLRLSCAASGFRFSSYAMSWVRQAPGKGLE
WVSKINSGGGITYYADSVKGRFTISRDNAKNTLYLQMNSLRAEDTAVYYCRDEG
PFGSWGQGTQVTVSS (SEQ ID NO: 34)
QVQLVESGGGLVQPGGSLRLSCAASGFTFSSYAMSWVRQAPGKGLE
WVSDINSGGSETYYADSVKGRFTISRDNAKNTLYLQMNSLRAEDTAVYYCRDK
GPFGSWGQGTQVTVSS (SEQ ID NO: 35)
EVQLVESGGGLVQPGGSLRLSCAASGRTFSSYDIIWVRQAPGKGLEFV
SAISTSGEHTYYADSVKGRFTISRDNAKNTLYLQMNSLRAEDTAVYYCNAVKWS
DYGPQNDYWGQGTLVTVSS (SEQ ID NO: 36)
EVQLQESGGGLVQPGGSLRLSCAASGFTLDYYAIGWFRQAPGKEREG
VSVIGKSDGSTCYADSVKGRFTISRDNAKNTVWLQMNSLRAEDTAVYYCAARP
WWVGDRPSIAAEYEYDLWGQGTQVTVSS (SEQ ID NO: 37)
MHSSALLCCLVLLTGVRAQVQLQESGPGLVRPSQTLSLTCTVSGYSIT
SDHAWSWVRQPPGRGLEWIGYISYSGITTYNPSLKSRVTMLRDTSKNQFSLRLSS
VTAADTAVYYCARSLARTTAMDYWGQGSLVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGSGGGGSEVQLVESGG
GLVQPGGSLRLSCAASGLYFSSYDIAWVRQAPGKGLEFVSAIDVSGEHTYYADSV
KGRFTISRDNAKNTLYLQMNSLRAEDTAVYYCNAVKWSDYGPDNDYWGQGTL
VTVSS (SEQ ID NO: 38)
MHSSALLCCLVLLTGVRASDTGRPFVEMYSEIPEIIHMTEGRELVIPCR
VTSPNITVTLKKFPLDTLIPDGKRIIWDSRKGFIISNATYKEIGLLTCEATVNGHLY
KTNYLTHRQTNTIIDVVLSPSHGIELSVGEKLVLNCTARTELNVGIDENWEYPSSK
HQHKKLVNRDLKTQSGSEMKKFLSTLTIDGVTRSDQGLYTCAASSGLMTKKNST
FVRVHEKDIQMTQSPSSLSASVGDRVTITCRASQDISSYLNWYQQKPGKAPKLLI
YYTSRLHSGVPSRFSGSGSGTDFTFTISSLQPEDIATYYCQQGNTLPYTFGQGTKV
EIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
(SEQ ID NO: 39)
MHSSALLCCLVLLTGVRAQVQLQESGPGLVRPSQTLSLTCTVSGYSIT
SDHAWSWVRQPPGRGLEWIGYISYSGITTYNPSLKSRVTMLRDTSKNQFSLRLSS
VTAADTAVYYCARSLARTTAMDYWGQGSLVTVSSASTKGPSVFPLAPSSKSTSG
GTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGSGGGGSEVQLQESGG
GLVQPGGSLRLSCAASGFTLDAYAIGWFRQAPGKEREGVSTIGKSDGSTCYADSV
KGRFTISRDNAKNTVWLQMNSLRAEDTAVYYCAARPWWVGDAPSIAAEYEYDL
WGQGTQVTVSS (SEQ ID NO: 40)
MHSSALLCCLVLLTGVRASDTGRPFVEMYSEIPEIIHMTEGREL VIPCR
VTSPNITVTLKKFPLDTLIPDGKRIIWDSRKGFIISNATYKEIGLLTCEATVNGHLY
KTNYLTHRQTNTIIDVVLSPSHGIELSVGEKLVLNCTARTELNVGIDENWEYPSSK
HQHKKLVNRDLKTQSGSEMKKFLSTLTIDGVTRSDQGLYTCAASSGLMTKKNST
FVRVHEKDIQMTQSPSSLSASVGDRVTITCRASQDISSYLNWYQQKPGKAPKLLI
YYTSRLHSGVPSRFSGSGSGTDFTFTISSLQPEDIATYYCQQGNTLPYTFGQGTKV
EIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
(SEQ ID NO: 41)
EVQLVESGGGLVQAGGSLRLSCAASGDDLSSVDMGWYRQAPGKGLE
FVGVISRGGGSTYYVDSVQGRFTISRDNAKNTVYLQMNSLRAEDTAVYYCKTEIP
YFDDYWGQGTLVTVSS (SEQ ID NO: 42)
EVQLVESGGGLVQAGGSLRLSCAASGESLSSVDMGWYRQAPGKGLE
FVGVISRGGESTYYVDSVQGRFTISRDNAKNTVYLQMNSLRAEDTAVYYCKTEIP
FGDDYWGQGTLVTVSS (SEQ ID NO: 43)
EVQLVESGGGLVQAGGSLRLSCAASGGDLSSVDMGWYRQAPGKGLE
FVGVISRTGGSTYYVDSVQGRFTISRDNAKNTVYLQMNSLRAEDTAVYYCKTEIP
GGDDYWGQGTLVTVSS (SEQ ID NO: 44)
EVQLVESGGGLVQAGGSLRLSCAASGESLSSVDMGWYRQAPGKGLE
FVGVISRTGGSTYYVDSVQGRFTISRDNAKNTVYLQMNSLRAEDTAVYYCKTEIP
GGDDVWGQGTLVTVSS (SEQ ID NO: 45)
EVQLVESGGGLVQPGGSLRLSCAASGGTFIDLDMGWYRQAPGNERQ
FVGVISRTGGSTYYADSVQGRFTISRDNAKNTLYLQMNSLRAEDTATYYCKTEIP
GGDDYWGQGTLVTVSS (SEQ ID NO: 46)
EVQLVESGGGLVQPGGSLRLSCAASGGTFHLYDMGWYRQAPGNERQ
FVGVISRGGGSTYYADSVQGRFTISRDNAKNTLYLQMNSLRAEDTATYYCKTEIP
GFQDYWGQGTLVTVSS (SEQ ID NO: 47)
EVQLVESGGGLVQPGGSLRLSCAASGPEFIDYDMGWYRQAPGNERQ
FVGVISRGGGSTYYADSVQGRFTISRDNAKNTLYLQMNSLRAEDTATYYCKTEIP
FFDDYWGQGTLVTVSS (SEQ ID NO: 48)
EVQLVESGGGLVQPGGSLRLSCAASGGTFIDLDMGWYRQAPGNERQ
FVGVISRGGGSVYYADSVQGRFTISRDNAKNTLYLQMNSLRAEDTATYYCKTEIP
GGPDYWGQGTLVTVSS (SEQ ID NO: 49)
EVQLVESGGGLVQPGGSLRLSCAASGGTFIDYDMGWYRQAPGNERQ
FVGVISRGGGAVYYADSVQGRFTISRDNAKNTLYLQMNSLRAEDTATYYCKTEI
PFFDDYWGQGTLVTVSS (SEQ ID NO: 50)
QVQLVESGGGLVQPGGSLRLSCAASGPTFSNLDMGWYRQAPGKGLE
LVGVISRTGGSTYYADSVKGRFTISRDNAKNTLYLQMNSLRAEDTAVYYCKTEV
PFSDDYWGQGTLVTVSS (SEQ ID NO: 51)
QVQLVESGGGLVQPGGSLRLSCAASVPTFGDLDMGWYRQAPGKGLE
LVGVISRTGGSTYY ADSVKGRFTISRDNAKNTLYLQMNSLRAEDTAVYYCKTEV
PFGDDYWGQGTLVTVSS (SEQ ID NO: 52)
QVQLVESGGGLVQPGGSLRLSCAASGDTFSNLDMGWYRQAPGKGLE
LVGVISRTGGSTYY ADSVKGRFTISRDNAKNTLYLQMNSLRAEDTAVYYCKTEV
PWADDYWGQGTLVTVSS (SEQ ID NO: 53)
QVQLVESGGGLVQPGGSLRLSCAASVPTFSDVDMGWYRQAPGKGLE
LVGVISRTGGSTYYADSVKGRFTISRDNAKNTLYLQMNSLRAEDTAVYYCKTEV
PFADDYWGQGTLVTVSS (SEQ ID NO: 54)
QVQLVESGGGLVQPGGSLRLSCAASVPWFSNLDMGWYRQAPGKGLE
LVGVISRTGGMTYYADSVKGRFTISRDNAKNTLYLQMNSLRAEDTAVYYCKTEV
PGSDDYWGQGTLVTVSS (SEQ ID NO: 55)
QVQLVESGGGLVQPGGSLRLSCAASGATFSNLDMGWYRQAPGKGLE
LVGVISRTGGSTYY ADSVKGRFTISRDNAKNTLYLQMNSLRAEDTAVYYCKTEV
PFDDDYWGQGTLVTVSS (SEQ ID NO: 56)
MHSSALLCCLVLLTGVRAEVQLVESGGGLVQAGGSLRLSCAASGDD
LSSVDMGWYRQAPGKGLEFVGVISRGGGSTYYVDSVQGRFTISRDNAKNTVYLQ
MNSLRAEDTAVYYCKTEIPYFDDYWGQGTLVTVSSGGSGGGSGGGGSGGGGGS
GGGGSDTGRPFVEMYSEIPEIIHMTEGRELVIPCRVTSPNITVTLKKFPLDTLIPDG
KRIIWDSRKGFIISNATYKEIGLLTCEATVNGHLYKTNYLTHRQTNTIIDVVLSPSH
GIELSVGEKLVLNCTARTELNVGIDENWEYPSSKHQHKKLVNRDLKTQSGSEMK
KFLSTLTIDGVTRSDQGLYTCAASSGLMTKKNSTFVRVHEKDKTHTCPPCPAPEL
LGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGGGGGS
GGGGSAQTNFMPMDDLEQRLYEQFILQQGLE (SEQ ID NO: 57)
MHSSALLCCLVLLTGVRAEVQLVESGGGLVQAGGSLRLSCAASGGD
LSSVDMGWYRQAPGKGLEFVGVISRTGGSTYYVDSVQGRFTISRDNAKNTVYLQ
MNSLRAEDTAVYYCKTEIPGGDDYWGQGTLVTVSSGGSGGGSGGGGSGGGGGS
GGGGSDTGRPFVEMYSEIPEIIHMTEGRELVIPCRVTSPNITVTLKKFPLDTLIPDG
KRIIWDSRKGFIISNATYKEIGLLTCEATVNGHLYKTNYLTHRQTNTIIDVVLSPSH
GIELSVGEKLVLNCTARTELNVGIDENWEYPSSKHQHKKLVNRDLKTQSGSEMK
KFLSTLTIDGVTRSDQGLYTCAASSGLMTKKNSTFVRVHEKDKTHTCPPCPAPEL
LGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGGGGGS
GGGGSAQTNFMPMDDLEQRLYEQFILQQGLE (SEQ ID NO: 58)
MHSSALLCCLVLLTGVRAAQTNFMPMDDLEQRLYEQFILQQGLEGG
GGSGGGGSGGGGSSDTGRPFVEMYSEIPEIIHMTEGRELVIPCRVTSPNITVTLKKF
PLDTLIPDGKRIIWDSRKGFIISNATYKEIGLLTCEATVNGHLYKTNYLTHRQTNTI
IDVVLSPSHGIELSVGEKLVLNCTARTELNVGIDENWEYPSSKHQHKKLVNRDLK
TQSGSEMKKFLSTLTIDGVTRSDQGLYTCAASSGLMTKKNSTFVRVHEKDKTHT
CPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLS
LSPGGGGGSGGGGSGGGGSEVQLVESGGGLVQAGGSLRLSCAASGDDLSSVDM
GWYRQAPGKGLEFVGVISRGGGSTYYVDSVQGRFTISRDNAKNTVYLQMNSLR
AEDTAVYYCKTEIPYFDDYWGQGTLVTVSS (SEQ ID NO: 59)
MHSSALLCCLVLLTGVRAAQTNFMPMDDLEQRLYEQFILQQGLEGG
GGSGGGGSGGGGSSDTGRPFVEMYSEIPEIIHMTEGRELVIPCRVTSPNITVTLKKF
PLDTLIPDGKRIIWDSRKGFIISNATYKEIGLLTCEATVNGHLYKTNYLTHRQTNTI
IDVVLSPSHGIELSVGEKLVLNCTARTELNVGIDENWEYPSSKHQHKKLVNRDLK
TQSGSEMKKFLSTLTIDGVTRSDQGLYTCAASSGLMTKKNSTFVRVHEKDKTHT
CPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLS
LSPGGGGGSGGGGSGGGGSEVQLVESGGGLVQAGGSLRLSCAASGGDLSSVDM
GWYRQAPGKGLEFVGVISRTGGSTYYVDSVQGRFTISRDNAKNTVYLQMNSLRA
EDTAVYYCKTEIPGGDDYWGQGTLVTVSS (SEQ ID NO: 60)
MHSSALLCCLVLLTGVRAEVQLVESGGGLVQPGGSLRLSCAASGLYF
SSYDIAWVRQAPGKGLEFVSAIDVSGEHTYYADSVKGRFTISRDNAKNTLYLQM
NSLRAEDTAVYYCNAVKWSDYGPDNDYWGQGTLVTVSSGGGGSGGGGSGGGG
SSDTGRPFVEMYSEIPEIIHMTEGRELVIPCRVTSPNITVTLKKFPLDTLIPDGKRIIW
DSRKGFIISNATYKEIGLLTCEATVNGHLYKTNYLTHRQTNTIIDVVLSPSHGIELS
VGEKLVLNCTARTELNVGIDENWEYPSSKHQHKKLVNRDLKTQSGSEMKKFLST
LTIDGVTRSDQGLYTCAASSGLMTKKNSTFVRVHEKDKTHTCPPCPAPELLGGPS
VFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGGGGGSGGGGS
GGGGSEVQLVESGGGLVQAGGSLRLSCAASGDDLSSVDMGWYRQAPGKGLEFV
GVISRGGGSTYYVDSVQGRFTISRDNAKNTVYLQMNSLRAEDTAVYYCKTEIPYF
DDYWGQGTLVTVSS (SEQ ID NO: 61)
MHSSALLCCLVLLTGVRAEVQLVESGGGLVQAGGSLRLSCAASGDD
LSSVDMGWYRQAPGKGLEFVGVISRGGGSTYYVDSVQGRFTISRDNAKNTVYLQ
MNSLRAEDTAVYYCKTEIPYFDDYWGQGTLVTVSSGGGGSGGGGSGGGGSSDT
GRPFVEMYSEIPEIIHMTEGRELVIPCRVTSPNITVTLKKFPLDTLIPDGKRIIWDSR
KGFIISNATYKEIGLLTCEATVNGHLYKTNYLTHRQTNTIIDVVLSPSHGIELSVGE
KLVLNCTARTELNVGIDFNWEYPSSKHQHKKLVNRDLKTQSGSEMKKFLSTLTI
DGVTRSDQGLYTCAASSGLMTKKNSTFVRVHEKDKTHTCPPCPAPELLGGPSVF
LFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGGGGGSGGGGSGG
GGSEVQLVESGGGLVQPGGSLRLSCAASGLYFSSYDIAWVRQAPGKGLEFVSAID
VSGEHTYYADSVKGRFTISRDNAKNTLYLQMNSLRAEDTAVYYCNAVKWSDYG
PDNDYWGQGTLVTVSS (SEQ ID NO: 62)
SDHAWS (SEQ ID NO: 63)
YISYSGITTYNPSLKS (SEQ ID NO: 64)
ARTTAMDY (SEQ ID NO: 65)
RASQDISSYLN (SEQ ID NO: 66)
YTSRLHS (SEQ ID NO: 67)
QQGNTLPYT (SEQ ID NO: 68)
EVQLVESGGGLVQPGRSLRLSCAASRFTFDDYAMHWVRQAPGKGLE
WVSGISWNSGRIGYADSVKGRFTISRDNAENSLFLQMNGLRAEDTALYYCAKGR
DSFDIWGQGTMVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEY
KCKVSNKALPAPIEKTISKAKGQPREPQVTYLPPSRDELTKNQVSLTCLVKGFYPS
DIAVEWESNGQPENNYKTTPPVLDSDGSFFLYSKLTVDKSRWQQGNVFSCSVMH
EALHNHYTQKSLSLSPGK (SEQ ID NO: 69)
DIQMTQSPSSVSASVGDRVTITCRASQGISSWLAWYQQKPGKAPKLLI
YGASSLESGVPSRFSGSGSGTDFTLTISSLQPEDFASYYCQQANSFPYTFGQGTKL
EIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
(SEQ ID NO: 70)
QVQLQESGPGLVKPSETLSLTCAVSGHSISHDHAWSWVRQPPGEGLE
WIGFISYSGITNYNPSLQGRVTISRDNSKNTLYLQMNSLRAEDTAVYYCARSLAR
TTAMDYWGEGTLVTVSSASTKGPSVFPLAPSSKSTSGGTAALGCLVKDYFPEPVT
VSWNSGALTSGVHTFPAVLQSSGLYSLSSVVTVPSSNFGTQTYTCNVDHKPSNTK
VDKTVERKSCVECPPCPAPPVAGPSVFLFPPKPKDTLMISRTPEVTCVVVDVSQED
PEVQFNWYVDGVEVHNAKTKPREEQFNSTFRVVSVLTVVHQDWLNGKEYKCK
VSNKGLPAPIEKTISKTKGQPREPQVYTLPPSQEEMTKNQVSLTCLVKGFYPSDIA
VEWESNGQPENNYKTTPPMLDSDGSFFLYSKLTVDKSRWQEGNVFSCSVMHEA
LHAHYTQKSLSLSP (SEQ ID NO: 71)
DIQMTQSPSSLSASVGDSVTITCQASTDISSHLNWYQQKPGKAPELLIY
YGSHLLSGVPSRFSGSGSGTDFTFTISSLEAEDAATYYCGQGNRLPYTFGQGTKVE
IERTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
(SEQ ID NO: 72)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
(SEQ ID NO: 73)
GACGTGGTGCTGAGCCCCAGCCACGGCATCGAGCTGAGCGTGGGC
GAGAAGCTGGTGCTGAACTGCACCGCCAGAACCGAGCTGAACGTGGGCATCG
ACTTCAACTGGGAGTACCCCAGCAGCAAGCACCAGCACAAGAAGCTGGTGAA
CAGAGACCTGAAGACCCAGAGCGGCAGCGAGATGAAGAAGTTCCTGAGCAC
CCTGACCATCGACGGCGTGACCAGAAGCGACCAGGGCCTGTACACCTGCGCC
GCCAGCAGCGGCCTGATGACCAAGAAGAACAGCACCTTCGTGAGAGTGCACG
AGAAG (SEQ ID NO: 74)
GTTGAGCCCAAATCTTGTGACAAAACTCACACATGCCCACCGTGC
CCAGCACCTGAACTCCTGGGGGGACCGTCAGTCTTCCTCTTCCCCCCAAAACC
CAAGGACACCCTCATGATCTCCCGGACCCCCGAGGTCACATGCGTGGTGGTG
GACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGA
AAACCATCTCCAAAGCCAAAGGGCAGCCCCGAGAACCACAGGTGTACACCCT
GCCCCCATCCCGGGAGGAGATGACCAAGAACCAGGTCAGCCTGACCTGCCTG
GTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTC
CTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCA
GAAGAGCCTCTCCCTGTCTCCG (SEQ ID 75)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCG (SEQ ID NO:
76)
GACATTCAGATGACCCAGAGCCCCAGCAGCCTGAGCGCCAGCGTG
GGAGACAGAGTGACCATCACCTGCAGAGCCAGCCAGGACATCTCCAGCTACC
TGAACTGGTATCAGCAGAAACCCGGCAAAGCCCCAAAACTGCTGATCTACTA
CACCAGTAGACTGCACAGCGGCGTGCCCAGCAGATTCTCAGGAAGCGGCTCC
GGAACCGACTTCACCTTCACTATCAGCAGCCTGCAGCCCGAAGATATTGCTAC
TTACTACTGCCAGCAGGGGAACACCCTGCCCTATACCTTCGGCCAGGGCACC
AAGGTGGAGATCAAACGTACGGTGGCTGCACCATCTGTCTTCATCTTCCCGCC
ATCTGATGAGCAGTTGAAATCTGGAACTGCCTCTGTTGTGTGCCTGCTGAATA
ACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACAC
AAAGTCTACGCCTGCGAAGTCACCCATCAGGGCCTGAGTTCGCCCGTCACAA
AGAGCTTCAACAGGGGAGAGTGTTGATTCTAGA (SEQ ID NO: 77)
AGTGCTCAGACCAATTTTATGCCTATGGATGATCTGGAACAGAGA
CTGTACGAACAGTTCATCCTCCAACAGGGACTG (SEQ ID NO: 78)
ATGGGCGCTCAGCAGAAGTTCCAGCCCCTGGACGAGCTGGAGCAG
ACCCTGTACGAGCAGTTCATGCTCCAGCAGGCCCTGGAG (SEQ ID NO: 79)
ATGGGCGCTCAGCAGAAGTACCAGCCCCTGGACGAGCTGGACAAG
ACCCTGTACGACCAGTTCATGCTCCAGCAGGGGCTGGAA (SEQ ID NO: 80)
ATGGGCGCTCAGCACACCTTCCAGCCCCTGGACGAGCTGGAGGAG
ACACTGTACTACCAGTGGCTCTACGACCAGCTGCTGGAG (SEQ ID NO: 81)
GCTCAGCAAGAAGAGTGTGAATGGGACCCTTGGACCTGTGAACAC
ATGCTGGAA (SEQ ID NO: 82)
GCTCAGACCAATATTCAAGAAGAATGTGAATGGGACCCTTGGACC
TGTGATCACATGCCCGGCAAACTGGAA (SEQ ID NO: 83)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGTGGCG
GATCAGGTGGGGGAGGCTCTGGTGGAGGCGGTAGTGCTCAGACCAATTTTAT
GCCTATGGATGATCTGGAACAGAGACTGTACGAACAGTTCATCCTCCAACAG
GGACTGGAA (SEQ ID NO: 84)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 85)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGAGGA
GGTTCTGGAGGTGGTGGATCAGCTCAGACCAATTTTATGCCTATGGATGATCT
GGAACAGAGACTGTACGAACAGTTCATCCTCCAACAGGGACTGGAA (SEQ ID
NO: 86)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 87)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGTGGCG
GATCAGGTGGGGGAGGCTCTGGTGGAGGCGGTAGTATGGGCGCTCAGCAGAA
GTTCCAGCCCCTGGACGAGCTGGAGCAGACCCTGTACGAGCAGTTCATGCTC
CAGCAGGCCCTGGAG (SEQ ID NO: 88)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 89)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGTGGCG
GATCAGGTGGGGGAGGCTCTGGTGGAGGCGGTAGTATGGGCGCTCAGCAGAA
GTACCAGCCCCTGGACGAGCTGGACAAGACCCTGTACGACCAGTTCATGCTC
CAGCAGGGGCTGGAA (SEQ ID NO: 90)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 91)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGAGGA
GGTTCTGGAGGTGGTGGATCAATGGGCGCTCAGCAGAAGTACCAGCCCCTGG
ACGAGCTGGACAAGACCCTGTACGACCAGTTCATGCTCCAGCAGGGGCTGGA
A (SEQ ID NO: 92)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 93)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGTGGCG
GATCAGGTGGGGGAGGCTCTGGTGGAGGCGGTAGTATGGGCGCTCAGCACAC
CTTCCAGCCCCTGGACGAGCTGGAGGAGACACTGTACTACCAGTGGCTCTAC
GACCAGCTGCTGGAG (SEQ ID NO: 94)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 95)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGTGGCG
GATCAGGTGGGGGAGGCTCTGGTGGAGGCGGTAGTGCTCAGCAAGAAGAGT
GTGAATGGGACCCTTGGACCTGTGAACACATGCTGGAA (SEQ ID NO: 96)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 97)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGTGGCG
GATCAGGTGGGGGAGGCTCTGGTGGAGGCGGTAGTGCTCAGACCAATATTCA
AGAAGAATGTGAATGGGACCCTTGGACCTGTGATCACATGCCCGGCAAACTG
GAA (SEQ ID NO: 98)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 99)
CAGGTGCAGCTGCAGGAGAGCGGCCCCGGACTGGTGAGGCCTTCC
CAGACCCTGAGCCTGACATGCACAGTGAGCGGCTACAGCATCACAAGCGACC
ACGCCTGGAGCTGGGTGAGACAGCCCCCCGGAAGAGGCCTGGAATGGATCGG
ATATATCTCCTACTCAGGCATTACCACCTACAACCCCTCTCTGAAAAGCAGAG
TGACCATGCTGAGAGATACTAGCAAGAACCAGTTTAGCCTGAGACTGTCTAG
CGTGACTGCCGCCGACACCGCCGTGTACTACTGCGCCAGATCCCTGGCCCGG
ACCACAGCCATGGATTACTGGGGCCAGGGAAGCCTGGTGACAGTGTCCTCCG
CTAGCACCAAGGGCCCATCGGTCTTCCCCCTGGCACCCTCCTCCAAGAGCACC
TCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCC
GAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCG
GGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCG
AGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGAGGA
GGTTCTGGAGGTGGTGGATCAGCTCAGACCAATATTCAAGAAGAATGTGAAT
GGGACCCTTGGACCTGTGATCACATGCCCGGCAAACTGGAA (SEQ ID NO: 100)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 101)
GAGGTGCAGCTGGTGGAGAGCGGCGGCGGACTGGTGCAGCCTGG
AGGATCTCTGAGGCTGTCCTGCGCCGCTTCTGGCCGGACCTTCTCCAGCTACG
ACTTTGTTTGGGTGCGGCAGGCCCCTGGCAAGGGACTGGAGTTCGTGTCCGCC
ATCTCCGTTGAGGGCGAGCACACCTACTATGCTGATAGCGTGAAGGGCCGGT
TCACCATCAGCAGGGATAACGCCAAGAATACCCTGTATCTGCAGATGAATAG
CCTGCGGGCTGAGGACACCGCCGTGTATTACTGTAACGCCGTGAAGTGGAGC
GACTACGGCCCCGACAACGATTACTGGGGCCAGGGCACCCTGGTGACCGTGT
CCTCC (SEQ ID NO: 102)
GAGGTGCAGCTGGTGGAGAGCGGCGGCGGACTGGTGCAGCCTGG
AGGATCTCTGAGGCTGTCCTGCGCCGCTTCTGGCCTTTATTTCTCCAGCTACG
ACATCGCTTGGGTGCGGCAGGCCCCTGGCAAGGGACTGGAGTTCGTGTCCGC
CATCGATGTGTCCGGCGAGCACACCTACTATGCTGATAGCGTGAAGGGCCGG
TTCACCATCAGCAGGGATAACGCCAAGAATACCCTGTATCTGCAGATGAATA
GCCTGCGGGCTGAGGACACCGCCGTGTATTACTGTAACGCCGTGAAGTGGAG
CGACTACGGCCCCGACAACGATTACTGGGGCCAGGGCACCCTGGTGACCGTG
TCCTCC (SEQ ID NO: 103)
GAGGTGCAGCTGCAGGAGAGCGGCGGCGGACTGGTGCAGCCAGG
AGGAAGCCTGCGGCTGTCCTGTGCCGCTTCCGGCTTTACCCTGGATGCGTATG
CTATCGGCTGGTTTAGGCAGGCCCCTGGCAAGGAGAGGGAGGGCGTGTCCAC
GATCGGCAAGTCCGACGGCAGCACCTGTTACGCCGACAGCGTGAAGGGCAGG
TTCACCATCAGCAGGGACAACGCTAAGAATACCGTGTGGCTGCAGATGAACT
CCCTGCGGGCTGAGGACACCGCCGTGTACTATTGCGCCGCTCGGCCCTGGTGG
GTGGGCGACGCGCCTTCTATCGCTGCCGAGTACGAGTACGATCTGTGGGGCC
AGGGCACCCAGGTGACCGTGAGCTCC (SEQ ID NO: 104)
GAGGTGCAGCTGCAGGAGAGCGGCGGCGGACTGGTGCAGCCAGG
AGGAAGCCTGCGGCTGTCCTGTGCCGCTTCTGGCTTTACCCTGGATGAGTATG
CTATCGGCTGGTTTAGGCAGGCCCCTGGCAAGGAGAGGGAGGGCGTGTCCTG
TATCGGCAAGGCGGACGGCAGCACCTGTTACGCCGACAGCGTGAAGGGCAGG
TTCACCATCAGCAGGGACAACGCTAAGAATACCGTGTGGCTGCAGATGAACT
CCCTGCGGGCTGAGGACACCGCCGTGTACTATTGCGCCGCTCGGCCCTGGTGG
GTGGGCGACCCTCCTTCTATCGGTGCCGAGTACGAGTACGATCTGTGGGGCCA
GGGCACCCAGGTGACCGTGAGCTCC (SEQ ID NO: 105)
CAGGTGCAGCTGGTGGAGAGCGGCGGCGGACTGGTGCAGCCTGGA
GGAAGCCTGCGGCTGTCCTGTGCCGCCAGCGGATTCAGGTTTAGCAGCTACG
CCATGTCCTGGGTGAGGCAGGCTCCTGGCAAGGGCCTGGAGTGGGTGAGCAA
GATCAATAGCGGCGGCGGCATCACCTACTACGCTGACTCCGTGAAGGGCAGG
TTTACCATCTCCAGGGATAACGCCAAGAATACCCTGTACCTGCAGATGAATA
GCCTGCGGGCTGAGGACACCGCCGTGTATTACTGTCGGGACGAGGGCCCTTTT
GGCAGCTGGGGCCAGGGCACCCAGGTGACAGTGAGCTCC (SEQ ID NO: 106)
CAGGTGCAGCTGGTGGAGAGCGGCGGCGGACTGGTGCAGCCTGGA
GGAAGCCTGCGGCTGTCCTGTGCCGCCAGCGGATTCACCTTTAGCAGCTACGC
CATGTCCTGGGTGAGGCAGGCTCCTGGCAAGGGCCTGGAGTGGGTGAGCGAC
ATCAATAGCGGCGGCAGTGAGACCTACTACGCTGACTCCGTGAAGGGCAGGT
TTACCATCTCCAGGGATAACGCCAAGAATACCCTGTACCTGCAGATGAATAG
CCTGCGGGCTGAGGACACCGCCGTGTATTACTGTCGGGACAAGGGCCCTTTTG
GCAGCTGGGGCCAGGGCACCCAGGTGACAGTGAGCTCC (SEQ ID NO: 107)
GAGGTGCAGCTGGTGGAGAGCGGCGGCGGACTGGTGCAGCCTGG
AGGATCTCTGAGGCTGTCCTGCGCCGCTTCTGGCCGGACCTTCTCCAGCTACG
ACATCATTTGGGTGCGGCAGGCCCCTGGCAAGGGACTGGAGTTCGTGTCCGC
CATCTCCACGTCCGGCGAGCACACCTACTATGCTGATAGCGTGAAGGGCCGG
TTCACCATCAGCAGGGATAACGCCAAGAATACCCTGTATCTGCAGATGAATA
GCCTGCGGGCTGAGGACACCGCCGTGTATTACTGTAACGCCGTGAAGTGGAG
CGACTACGGCCCCCAGAACGATTACTGGGGCCAGGGCACCCTGGTGACCGTG
TCCTCC (SEQ ID NO: 108)
GAGGTGCAGCTGCAGGAGAGCGGCGGCGGACTGGTGCAGCCAGG
AGGAAGCCTGCGGCTGTCCTGTGCCGCTTCCGGCTTTACCCTGGATTACTATG
CTATCGGCTGGTTTAGGCAGGCCCCTGGCAAGGAGAGGGAGGGCGTGTCCGT
GATCGGCAAGTCCGACGGCAGCACCTGTTACGCCGACAGCGTGAAGGGCAGG
TTCACCATCAGCAGGGACAACGCTAAGAATACCGTGTGGCTGCAGATGAACT
CCCTGCGGGCTGAGGACACCGCCGTGTACTATTGCGCCGCTCGGCCCTGGTGG
GTGGGCGACAGGCCTTCTATCGCTGCCGAGTACGAGTACGATCTGTGGGGCC
AGGGCACCCAGGTGACCGTGAGCTCC (SEQ ID NO: 109)
AGGTTCAGCTCCAGGAGTCAGGCCCAGGACTCGTTAGGCCATCC
CAGACACTCTCCCTCACATGCACAGTCTCCGGCTACTCCATCACATCAGACCA
CGCCTGGTCCTGGGTCAGGCAGCCTCCTGGAAGAGGACTCGAATGGATCGGA
TATATCTCCTACTCCGGCATTACCACCTACAACCCCTCACTGAAGTCCCGCGT
GACCATGTTGAGAGATACTAGCAAGAACCAGTTCTCCCTGAGACTGAGCTCC
GTGACTGCCGCTGACACCGCTGTTTACTATTGCGCCAGATCCCTGGCCCGGAC
CACAGCTATGGATTACTGGGGCCAGGGAAGCCTGGTGACCGTTTCTTCCGCTA
GCACCAAAGGTCCCAGCGTGTTCCCACTCGCCCCGAGTTCAAAATCAACTTCT
GGAGGCACCGCCGCCCTGGGTTGCCTGGTAAAGGACTACTTCCCAGAGCCCG
TGACCGTGAGCTGGAACTCCGGGGCACTGACATCTGGCGTTCATACTTTCCCG
GCCGTGCTCCAGTCTTCAGGTCTGTATAGTCTCTCCTCTGTGGTCACTGTCCCA
TCTAGCTCTCTGGGCACCCAAACCTACATATGCAACGTTAATCACAAGCCGAG
CAATACTAAAGTTGACAAAAAGGTGGAACCCAAGTCTTGTGACAAGACCCAC
ACGTGTCCCCCCTGCCCGGCTCCTGAGCTGCTTGGCGGCCCCAGCGTCTTTCT
CTTTCCCCCAAAGCCAAAAGATACCTTGATGATCAGCAGAACTCCCGAGGTG
ACATGCGTCGTCGTGGACGTAAGCCATGAAGATCCCGAGGTTAAGTTCAACT
GGTATGTCGATGGCGTGGAAGTCCATAATGCTAAGACTAAACCTCGCGAAGA
GCAGTACAATTCAACTTACCGGGTCGTTTCCGTTCTGACCGTGCTGCATCAGG
ACTGGCTGAATGGTAAAGAGTACAAGTGCAAAGTGTCTAACAAGGCACTCCC
CGCCCCAATTGAGAAGACTATCTCCAAAGCTAAAGGGCAACCAAGAGAGCCC
CAGGTCTACACCCTGCCCCCCTCAAGGGAAGAGATGACTAAGAACCAGGTTA
GTCTCACCTGCTTGGTTAAAGGATTTTATCCAAGCGATATTGCTGTGGAGTGG
GAGTCCAACGGCCAGCCTGAGAACAATTATAAAACCACCCCCCCTGTTCTTG
ACAGTGACGGTAGTTTCTTCCTGTATTCCAAACTGACCGTCGATAAGAGCAGA
TGGCAACAGGGAAATGTGTTCAGCTGCTCCGTGATGCATGAGGCGCTCCATA
ATCATTACACACAAAAAAGTTTGTCCCTGAGCCCAGGCGGAGGAGGAAGTGG
AGGAGGAGGAAGCGAGGTTCAGTTGGTGGAGTCCGGGGGAGGATTGGTGCA
GCCTGGAGGAAGTTTGAGGTTGAGCTGTGCCGCATCCGGGTTGTACTTTAGCA
GCTACGACATTGCATGGGTGAGGCAGGCACCTGGCAAGGGATTGGAGTTTGT
GAGCGCCATCGACGTGAGCGGAGAGCATACATACTACGCTGACAGTGTGAAG
GGCCGGTTTACCATCTCCAGGGACAACGCTAAGAACACACTGTACCTGCAGA
TGAACAGCCTGAGGGCAGAGGACACAGCAGTGTACTACTGCAACGCAGTGAA
ATGGAGCGATTACGGACCAGACAACGACTACTGGGGACAGGGAACCCTCGTG
ACCGTGTCTAGC (SEQ ID NO: 110)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID No: 111)
CAGGTTCAGCTCCAGGAGTCAGGCCCAGGACTCGTTAGGCCATCC
CAGACACTCTCCCTCACATGCACAGTCTCCGGCTACTCCATCACATCAGACCA
CGCCTGGTCCTGGGTCAGGCAGCCTCCTGGAAGAGGACTCGAATGGATCGGA
TATATCTCCTACTCCGGCATTACCACCTACAACCCCTCACTGAAGTCCCGCGT
GACCATGTTGAGAGATACTAGCAAGAACCAGTTCTCCCTGAGACTGAGCTCC
GTGACTGCCGCTGACACCGCTGTTTACTATTGCGCCAGATCCCTGGCCCGGAC
CACAGCTATGGATTACTGGGGCCAGGGAAGCCTGGTGACCGTTTCTTCCGCTA
GCACCAAAGGTCCCAGCGTGTTCCCACTCGCCCCGAGTTCAAAATCAACTTCT
GGAGGCACCGCCGCCCTGGGTTGCCTGGTAAAGGACTACTTCCCAGAGCCCG
TGACCGTGAGCTGGAACTCCGGGGCACTGACATCTGGCGTTCATACTTTCCCG
GCCGTGCTCCAGTCTTCAGGTCTGTATAGTCTCTCCTCTGTGGTCACTGTCCCA
TCTAGCTCTCTGGGCACCCAAACCTACATATGCAACGTTAATCACAAGCCGAG
CAATACTAAAGTTGACAAAAAGGTGGAACCCAAGTCTTGTGACAAGACCCAC
ACGTGTCCCCCCTGCCCGGCTCCTGAGCTGCTTGGCGGCCCCAGCGTCTTTCT
CTTTCCCCCAAAGCCAAAAGATACCTTGATGATCAGCAGAACTCCCGAGGTG
ACATGCGTCGTCGTGGACGTAAGCCATGAAGATCCCGAGGTTAAGTTCAACT
GGTATGTCGATGGCGTGGAAGTCCATAATGCTAAGACTAAACCTCGCGAAGA
GCAGTACAATTCAACTTACCGGGTCGTTTCCGTTCTGACCGTGCTGCATCAGG
ACTGGCTGAATGGTAAAGAGTACAAGTGCAAAGTGTCTAACAAGGCACTCCC
CGCCCCAATTGAGAAGACTATCTCCAAAGCTAAAGGGCAACCAAGAGAGCCC
CAGGTCTACACCCTGCCCCCCTCAAGGGAAGAGATGACTAAGAACCAGGTTA
GTCTCACCTGCTTGGTTAAAGGATTTTATCCAAGCGATATTGCTGTGGAGTGG
GAGTCCAACGGCCAGCCTGAGAACAATTATAAAACCACCCCCCCTGTTCTTG
ACAGTGACGGTAGTTTCTTCCTGTATTCCAAACTGACCGTCGATAAGAGCAGA
TGGCAACAGGGAAATGTGTTCAGCTGCTCCGTGATGCATGAGGCGCTCCATA
ATCATTACACACAAAAAAGTTTGTCCCTGAGCCCAGGCGGAGGAGGAAGTGG
AGGAGGAGGAAGCGAGGTTCAGTTGCAGGAGAGCGGGGGAGGATTGGTGCA
GCCTGGAGGAAGTTTGAGGTTGAGCTGTGCCGCATCCGGGTTTACATTGGAC
GCATACGCTATTGGATGGTTCAGGCAGGCACCTGGCAAGGAGAGGGAGGGA
GTTAGCACTATCGGCAAGAGTGACGGCAGCACATGCTACGCCGACTCCGTTA
AGGGCAGGTTTACCATTTCCCGGGACAACGCCAAGAACACCGTGTGGCTGCA
GATGAACAGCCTGAGGGCCGAGGACACAGCAGTGTATTATTGCGCCGCCAGG
CCCTGGTGGGTTGGAGACGCTCCTTCTATCGCCGCCGAGTACGAGTACGACCT
GTGGGGACAGGGCACCCAGGTTACTGTGTCCAGC (SEQ ID NO: 112)
AGCGACACCGGCAGACCCTTCGTGGAGATGTACAGCGAGATCCCC
GAGATCATCCACATGACCGAGGGCAGAGAGCTGGTGATCCCCTGCAGAGTGA
CCAGCCCCAACATCACCGTGACCCTGAAGAAGTTCCCCCTGGACACCCTGATC
CCCGACGGCAAGAGAATCATCTGGGACAGCAGAAAGGGCTTCATCATCAGCA
ACGCCACCTACAAGGAGATCGGCCTGCTGACCTGCGAGGCCACCGTGAACGG
CCACCTGTACAAGACCAACTACCTGACCCACAGACAGACCAACACCATCATC
GACGTGGTGCTGAGCCCCAGCCACGGCATCGAGCTGAGCGTGGGCGAGAAGC
TGGTGCTGAACTGCACCGCCAGAACCGAGCTGAACGTGGGCATCGACTTCAA
CTGGGAGTACCCCAGCAGCAAGCACCAGCACAAGAAGCTGGTGAACAGAGA
CCTGAAGACCCAGAGCGGCAGCGAGATGAAGAAGTTCCTGAGCACCCTGACC
ATCGACGGCGTGACCAGAAGCGACCAGGGCCTGTACACCTGCGCCGCCAGCA
GCGGCCTGATGACCAAGAAGAACAGCACCTTCGTGAGAGTGCACGAGAAGG
ACATTCAGATGACCCAGAGCCCCAGCAGCCTGAGCGCCAGCGTGGGAGACAG
AGTGACCATCACCTGCAGAGCCAGCCAGGACATCTCCAGCTACCTGAACTGG
TATCAGCAGAAACCCGGCAAAGCCCCAAAACTGCTGATCTACTACACCAGTA
GACTGCACAGCGGCGTGCCCAGCAGATTCTCAGGAAGCGGCTCCGGAACCGA
CTTCACCTTCACTATCAGCAGCCTGCAGCCCGAAGATATTGCTACTTACTACT
GCCAGCAGGGGAACACCCTGCCCTATACCTTCGGCCAGGGCACCAAGGTGGA
GATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTA
ACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTAC
GCCTGCGAAGTCACCCATCAGGGCCTGAGTTCGCCCGTCACAAAGAGCTTCA
ACAGGGGAGAGTGT (SEQ ID NO: 113)
GAGGTGCAGTTGGTGGAGTCTGGGGGAGGATTGGTGCAGGCTGGG
GGCTCACTGAGACTCTCCTGTGCAGCCTCTGGAGATGATTTAAGTAGCGTAGA
CATGGGCTGGTACCGCCAGGCTCCAGGGAAGGGCCTGGAGTTTGTCGGCGTT
ATTAGCCGTGGTGGTGGTAGCACATACTATGTAGACTCCGTGCAGGGCCGATT
CACCATCTCCAGAGACAACGCCAAGAACACGGTGTATCTGCAAATGAACAGC
CTGCGGGCCGAGGACACGGCCGTGTATTACTGTAAGACAGAAATTCCATATT
TTGATGACTACTGGGGCCAGGGGACCCTGGTCACCGTCTCCTCA (SEQ ID NO:
114)
GAGGTGCAGTTGGTGGAGTCTGGGGGAGGATTGGTGCAGGCTGGG
GGCTCACTGAGACTCTCCTGTGCAGCCTCTGGAGAGTCCTTAAGTAGCGTAGA
CATGGGCTGGTACCGCCAGGCTCCAGGGAAGGGCCTGGAGTTTGTCGGCGTT
ATTAGCCGTGGTGGTGAGAGCACATACTATGTAGACTCCGTGCAGGGCCGAT
TCACCATCTCCAGAGACAACGCCAAGAACACGGTGTATCTGCAAATGAACAG
CCTGCGGGCCGAGGACACGGCCGTGTATTACTGTAAGACAGAAATTCCATTT
GGGGATGACTACTGGGGCCAGGGGACCCTGGTCACCGTCTCCTCA (SEQ ID
NO: 115)
GAGGTGCAGTTGGTGGAGTCTGGGGGAGGATTGGTGCAGGCTGGG
GGCTCACTGAGACTCTCCTGTGCAGCCTCTGGAGGTGATTTAAGTAGCGTAGA
CATGGGCTGGTACCGCCAGGCTCCAGGGAAGGGCCTGGAGTTTGTCGGCGTT
ATTAGCAGGACGGGTGGTAGCACATACTATGTAGACTCCGTGCAGGGCCGAT
TCACCATCTCCAGAGACAACGCCAAGAACACGGTGTATCTGCAAATGAACAG
CCTGCGGGCCGAGGACACGGCCGTGTATTACTGTAAGACAGAAATTCCAGGG
GGGGATGACTACTGGGGCCAGGGGACCCTGGTCACCGTCTCCTCA (SEQ ID
NO: 116)
GAGGTGCAGTTGGTGGAGTCTGGGGGAGGATTGGTGCAGGCTGGG
GGCTCACTGAGACTCTCCTGTGCAGCCTCTGGAGAGTCCTTAAGTAGCGTAGA
CATGGGCTGGTACCGCCAGGCTCCAGGGAAGGGCCTGGAGTTTGTCGGCGTT
ATTAGCCGTACGGGTGGTAGCACATACTATGTAGACTCCGTGCAGGGCCGAT
TCACCATCTCCAGAGACAACGCCAAGAACACGGTGTATCTGCAAATGAACAG
CCTGCGGGCCGAGGACACGGCCGTGTATTACTGTAAGACAGAAATTCCAGGG
GGGGATGACGTTTGGGGCCAGGGGACCCTGGTCACCGTCTCCTCA (SEQ ID
NO: 117)
GAGGTGCAGTTGGTGGAGAGCGGGGGGGGGCTGGTGCAGCCTGG
AGGAAGTTTGAGGTTGAGCTGTGCCGCAAGCGGGGGGACATTCATTGATTTG
GACATGGGATGGTACAGGCAGGCACCTGGAAACGAGAGGCAGTTTGTGGGG
GTGATTTCCCGGACTGGAGGATCCACATACTACGCTGACAGCGTGCAGGGGA
GGTTCACCATCTCCCGGGACAACGCCAAGAACACCCTGTACCTGCAGATGAA
CTCCCTGAGGGCTGAGGACACTGCTACCTATTACTGCAAGACCGAGATTCCCG
GAGGAGACGACTACTGGGGCCAGGGCACACTGGTGACCGTGAGCTCC (SEQ
ID NO: 118)
GAGGTGCAGTTGGTGGAGAGCGGGGGGGGGCTGGTGCAGCCTGG
AGGAAGTTTGAGGTTGAGCTGTGCCGCAAGCGGGGGGACATTCCATCTGTAC
GACATGGGATGGTACAGGCAGGCACCTGGAAACGAGAGGCAGTTTGTGGGG
GTGATTTCCCGCGGCGGAGGATCCACATACTACGCTGACAGCGTGCAGGGGA
GGTTCACCATCTCCCGGGACAACGCCAAGAACACCCTGTACCTGCAGATGAA
CTCCCTGAGGGCTGAGGACACTGCTACCTATTACTGCAAGACCGAGATTCCCG
GATTTCAGGACTACTGGGGCCAGGGCACACTGGTGACCGTGAGCTCC (SEQ ID
NO: 119)
GAGGTGCAGTTGGTGGAGAGCGGGGGGGGGCTGGTGCAGCCTGG
AGGAAGTTTGAGGTTGAGCTGTGCCGCAAGCGGGCCGGAGTTCATTGATTAC
GACATGGGATGGTACAGGCAGGCACCTGGAAACGAGAGGCAGTTTGTGGGG
GTGATTTCCCGCGGCGGAGGATCCACATACTACGCTGACAGCGTGCAGGGGA
GGTTCACCATCTCCCGGGACAACGCCAAGAACACCCTGTACCTGCAGATGAA
CTCCCTGAGGGCTGAGGACACTGCTACCTATTACTGCAAGACCGAGATTCCCT
TTTTTGACGACTACTGGGGCCAGGGCACACTGGTGACCGTGAGCTCC (SEQ ID
NO: 120)
GAGGTGCAGTTGGTGGAGAGCGGGGGGGGGCTGGTGCAGCCTGG
AGGAAGTTTGAGGTTGAGCTGTGCCGCAAGCGGGGGGACATTCATTGATCTG
GACATGGGATGGTACAGGCAGGCACCTGGAAACGAGAGGCAGTTTGTGGGG
GTGATTTCCCGCGGCGGAGGATCCGTTTACTACGCTGACAGCGTGCAGGGGA
GGTTCACCATCTCCCGGGACAACGCCAAGAACACCCTGTACCTGCAGATGAA
CTCCCTGAGGGCTGAGGACACTGCTACCTATTACTGCAAGACCGAGATTCCCG
GAGGACCTGACTACTGGGGCCAGGGCACACTGGTGACCGTGAGCTCC (SEQ
ID NO: 121)
GAGGTGCAGTTGGTGGAGAGCGGGGGGGGGCTGGTGCAGCCTGG
AGGAAGTTTGAGGTTGAGCTGTGCCGCAAGCGGGGGGACATTCATTGATTAC
GACATGGGATGGTACAGGCAGGCACCTGGAAACGAGAGGCAGTTTGTGGGG
GTGATTTCCCGCGGCGGAGGAGCTGTTTACTACGCTGACAGCGTGCAGGGGA
GGTTCACCATCTCCCGGGACAACGCCAAGAACACCCTGTACCTGCAGATGAA
CTCCCTGAGGGCTGAGGACACTGCTACCTATTACTGCAAGACCGAGATTCCCT
TTTTTGACGACTACTGGGGCCAGGGCACACTGGTGACCGTGAGCTCC (SEQ ID
NO: 122)
CAGGTCCAGCTCGTCGAGTCAGGCGGCGGCCTCGTGCAGCCTGGA
GGAAGTTTGAGGTTGAGTTGTGCAGCCAGCGGTCCTACATTTTCCAACCTCGA
CATGGGCTGGTACAGGCAGGCACCCGGCAAAGGGCTGGAGCTGGTGGGCGTT
ATTTCTCGGACAGGAGGGTCCACATACTACGCCGATTCCGTGAAGGGCAGAT
TCACCATCAGCAGGGACAATGCTAAGAACACCCTGTACCTGCAGATGAACTC
CTTGAGGGCTGAGGACACCGCAGTGTACTACTGCAAGACCGAGGTGCCCTTT
TCTGACGATTACTGGGGACAGGGCACACTGGTGACCGTGAGCTCC (SEQ ID
NO: 123)
CAGGTCCAGCTCGTCGAGTCAGGCGGCGGCCTCGTGCAGCCTGGA
GGAAGTTTGAGGTTGAGTTGTGCAGCCAGCGTGCCGACATTTGGGGATCTCG
ACATGGGCTGGTACAGGCAGGCACCCGGCAAAGGGCTGGAGCTGGTGGGCGT
TATTTCTCGGACAGGAGGGTCCACATACTACGCCGATTCCGTGAAGGGCAGA
TTCACCATCAGCAGGGACAATGCTAAGAACACCCTGTACCTGCAGATGAACT
CCTTGAGGGCTGAGGACACCGCAGTGTACTACTGCAAGACCGAGGTGCCCTT
TGGGGACGATTACTGGGGACAGGGCACACTGGTGACCGTGAGCTCC (SEQ ID
NO: 124)
CAGGTCCAGCTCGTCGAGTCAGGCGGCGGCCTCGTGCAGCCTGGA
GGAAGTTTGAGGTTGAGTTGTGCAGCCAGCGGGGATACATTTTCCAACCTCG
ACATGGGCTGGTACAGGCAGGCACCCGGCAAAGGGCTGGAGCTGGTGGGCGT
TATTTCTCGGACAGGAGGGTCCACATACTACGCCGATTCCGTGAAGGGCAGA
TTCACCATCAGCAGGGACAATGCTAAGAACACCCTGTACCTGCAGATGAACT
CCTTGAGGGCTGAGGACACCGCAGTGTACTACTGCAAGACCGAGGTGCCCTG
GGCGGACGATTACTGGGGACAGGGCACACTGGTGACCGTGAGCTCC (SEQ ID
NO: 125)
CAGGTCCAGCTCGTCGAGTCAGGCGGCGGCCTCGTGCAGCCTGGA
GGAAGTTTGAGGTTGAGTTGTGCAGCCAGCGTGCCGACATTTTCCGATGTGGA
CATGGGCTGGTACAGGCAGGCACCCGGCAAAGGGCTGGAGCTGGTGGGCGTT
ATTTCTCGGACAGGAGGGTCCACATACTACGCCGATTCCGTGAAGGGCAGAT
TCACCATCAGCAGGGACAATGCTAAGAACACCCTGTACCTGCAGATGAACTC
CTTGAGGGCTGAGGACACCGCAGTGTACTACTGCAAGACCGAGGTGCCCTTT
GCTGACGATTACTGGGGACAGGGCACACTGGTGACCGTGAGCTCC (SEQ ID
NO: 126)
CAGGTCCAGCTCGTCGAGTCAGGCGGCGGCCTCGTGCAGCCTGGA
GGAAGTTTGAGGTTGAGTTGTGCAGCCAGCGTGCCGTGGTTTTCCAACCTCGA
CATGGGCTGGTACAGGCAGGCACCCGGCAAAGGGCTGGAGCTGGTGGGCGTT
ATTTCTCGGACAGGAGGGATGACATACTACGCCGATTCCGTGAAGGGCAGAT
TCACCATCAGCAGGGACAATGCTAAGAACACCCTGTACCTGCAGATGAACTC
CTTGAGGGCTGAGGACACCGCAGTGTACTACTGCAAGACCGAGGTGCCCGGC
TCGGACGATTACTGGGGACAGGGCACACTGGTGACCGTGAGCTCC (SEQ ID
NO: 127)
CAGGTCCAGCTCGTCGAGTCAGGCGGCGGCCTCGTGCAGCCTGGA
GGAAGTTTGAGGTTGAGTTGTGCAGCCAGCGGGGCGACATTTTCCAACCTCG
ACATGGGCTGGTACAGGCAGGCACCCGGCAAAGGGCTGGAGCTGGTGGGCGT
TATTTCTCGGACAGGAGGGTCCACATACTACGCCGATTCCGTGAAGGGCAGA
TTCACCATCAGCAGGGACAATGCTAAGAACACCCTGTACCTGCAGATGAACT
CCTTGAGGGCTGAGGACACCGCAGTGTACTACTGCAAGACCGAGGTGCCCTT
TGATGACGATTACTGGGGACAGGGCACACTGGTGACCGTGAGCTCC (SEQ ID
NO: 128)
GAGGTTCAGTTGGTGGAGTCCGGGGGAGGATTGGTGCAGGCTGGA
GGAAGTTTGAGGTTGTCCTGTGCCGCCAGCGGGGATGATTTGAGCTCCGTTGA
CATGGGATGGTACAGGCAGGCACCCGGAAAGGGACTGGAGTTCGTGGGAGT
GATTAGCAGGGGGGGAGGAAGCACATACTACGTGGACTCCGTGCAGGGAAG
GTTTACAATCTCCCGGGACAACGCTAAGAACACAGTGTACCTGCAGATGAAC
AGCCTCAGGGCCGAGGACACAGCAGTGTACTACTGCAAAACCGAGATTCCCT
ATTTTGACGATTACTGGGGCCAGGGCACACTGGTGACCGTTAGTTCCGGAGG
ATCTGGAGGAGGAAGCGGAGGAGGAGGAAGTGGAGGTGGAGGAGGTTCTGG
AGGTGGTGGATCAGACACAGGAAGGCCTTTTGTTGAGATGTATAGCGAGATT
CCCGAGATCATTCACATGACCGAAGGGCGGGAGCTGGTGATTCCCTGTAGGG
TTACCAGCCCCAACATCACCGTGACCCTGAAGAAGTTTCCCCTGGACACACTG
ATCCCCGACGGCAAAAGAATCATCTGGGACTCCCGGAAGGGATTTATCATTT
CCAACGCCACCTACAAGGAGATCGGCCTCCTGACCTGCGAAGCAACCGTGAA
CGGCCACCTCTATAAGACCAACTACCTGACACACCGGCAGACCAACACAATC
ATTGACGTGGTGCTCTCCCCCTCCCACGGAATTGAGCTGTCCGTTGGCGAGAA
ACTGGTGCTGAACTGCACCGCCAGGACCGAACTGAACGTGGGAATCGACTTC
AACTGGGAATACCCCTCCAGCAAGCACCAGCACAAGAAGCTGGTGAACAGG
GACCTGAAAACCCAGAGCGGCTCCGAAATGAAAAAGTTCCTGTCCACCCTGA
CCATCGACGGCGTGACCAGGTCCGACCAGGGCCTGTACACCTGCGCCGCCTC
CTCCGGCCTGATGACCAAGAAGAACTCCACCTTCGTGAGGGTGCACGAGAAG
GACAAAACTCACACATGCCCACCGTGCCCAGCACCTGAACTCCTGGGGGGAC
CGTCAGTCTTCCTCTTCCCCCCAAAACCCAAGGACACCCTCATGATCTCCCGG
ACCCCCGAGGTCACATGCGTGGTGGTGGACGTGAGCCACGAAGACCCTGAGG
TCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACC
GTCCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGGCA
GCCCCGAGAACCACAGGTGTACACCCTGCCCCCATCCCGGGACGAGCTGACC
AAGAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACCA
CGCCTCCCGTGCTGGACTCCGACGGCTCCTTCTTCCTCTACAGCAAGCTCACC
GTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGT
GGTGGAGGAGGTTCTGGAGGTGGTGGATCAGCTCAGACAAATTTCATGCCTA
TGGATGATTTGGAGCAGAGGCTGTACGAACAGTTCATCCTGCAGCAGGGCCT
GGAA (SEQ ID NO: 129)
GAGGTTCAGTTGGTGGAGTCCGGGGGAGGATTGGTGCAGGCTGGA
GGAAGTTTGAGGTTGTCCTGTGCCGCCAGCGGGGGAGACTTGTCCTCCGTGG
ACATGGGATGGTACAGGCAGGCCCCTGGAAAGGGATTGGAGTTTGTGGGGGT
TATTTCCCGGACTGGAGGGTCCACCTATTATGTTGACAGTGTGCAGGGACGGT
TTACCATTTCTAGGGACAACGCTAAGAACACAGTTTACCTGCAGATGAATAG
CTTGAGGGCTGAGGACACTGCTGTGTATTATTGCAAGACCGAGATTCCAGGA
GGAGACGACTACTGGGGCCAGGGAACACTGGTGACCGTGAGTTCCGGAGGAT
CTGGAGGAGGAAGCGGAGGAGGAGGAAGTGGAGGTGGAGGAGGTTCTGGAG
GTGGTGGATCAGACACAGGAAGGCCTTTTGTTGAGATGTATAGCGAGATTCC
CGAGATCATTCACATGACCGAAGGGCGGGAGCTGGTGATTCCCTGTAGGGTT
ACCAGCCCCAACATCACCGTGACCCTGAAGAAGTTTCCCCTGGACACACTGA
TCCCCGACGGCAAAAGAATCATCTGGGACTCCCGGAAGGGATTTATCATTTCC
AACGCCACCTACAAGGAGATCGGCCTCCTGACCTGCGAAGCAACCGTGAACG
GCCACCTCTATAAGACCAACTACCTGACACACCGGCAGACCAACACAATCAT
TGACGTGGTGCTCTCCCCCTCCCACGGAATTGAGCTGTCCGTTGGCGAGAAAC
TGGTGCTGAACTGCACCGCCAGGACCGAACTGAACGTGGGAATCGACTTCAA
CTGGGAATACCCCTCCAGCAAGCACCAGCACAAGAAGCTGGTGAACAGGGAC
CTGAAAACCCAGAGCGGCTCCGAAATGAAAAAGTTCCTGTCCACCCTGACCA
TCGACGGCGTGACCAGGTCCGACCAGGGCCTGTACACCTGCGCCGCCTCCTCC
GGCCTGATGACCAAGAAGAACTCCACCTTCGTGAGGGTGCACGAGAAGGACA
AAACTCACACATGCCCACCGTGCCCAGCACCTGAACTCCTGGGGGGACCGTC
AGTCTTCCTCTTCCCCCCAAAACCCAAGGACACCCTCATGATCTCCCGGACCC
CCGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCG
CGGGAGGAGCAGTACAACAGCACGTACCGTGTGGTCAGCGTCCTCACCGTCC
TGCACCAGGACTGGCTGAATGGCAAGGAGTACAAGTGCAAGGTCTCCAACAA
AGCCCTCCCAGCCCCCATCGAGAAAACCATCTCCAAAGCCAAAGGGCAGCCC
CGAGAACCACAGGTGTACACCCTGCCCCCATCCCGGGACGAGCTGACCAAGA
ACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCT
CCCGTGCTGGACTCCGACGGCTCCTTCTTCCTCTACAGCAAGCTCACCGTGGA
CAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTGGTGG
AGGAGGTTCTGGAGGTGGTGGATCAGCTCAGACAAATTTCATGCCTATGGAT
GATTTGGAGCAGAGGCTGTACGAACAGTTCATCCTGCAGCAGGGCCTGGAA
(SEQ ID NO: 130)
GCTCAGACAAATTTCATGCCTATGGATGATTTGGAGCAGAGGCTG
TACGAACAGTTCATCCTGCAGCAGGGCCTGGAAGGTGGTGGCGGATCAGGTG
GGGGAGGCTCTGGTGGAGGCGGTAGTTCCGATACCGGTCGTCCTTTTGTCGAG
ATGTACTCCGAGATCCCCGAGATTATTCACATGACCGAGGGCCGCGAGCTGG
TGATTCCTTGTCGTGTGACCTCCCCTAACATCACCGTTACCCTGAAGAAGTTT
CCCCTGGATACACTGATCCCTGATGGCAAGAGGATTATCTGGGACTCCAGGA
AGGGATTCATTATTTCCAACGCCACCTACAAGGAGATCGGCCTCCTGACCTGC
GAAGCCACAGTTAACGGCCACCTCTACAAGACCAACTACCTGACACACCGGC
AGACCAACACCATCATCGACGTCGTGCTCAGCCCCTCCCACGGAATTGAGCT
GTCCGTCGGAGAAAAGCTGGTGCTGAACTGCACCGCCAGAACCGAACTGAAC
GTGGGCATCGACTTCAACTGGGAGTACCCCTCCTCCAAACACCAGCACAAGA
AACTCGTCAACAGGGACCTGAAAACTCAATCTGGCTCCGAAATGAAAAAGTT
CCTGTCCACCCTGACCATCGACGGCGTGACCAGGTCCGACCAGGGCCTGTAC
ACCTGCGCCGCCTCCTCCGGCCTGATGACCAAGAAGAACTCCACCTTCGTGAG
GGTGCACGAGAAGGACAAAACTCACACATGCCCACCGTGCCCAGCACCTGAA
CTCCTGGGGGGACCGTCAGTCTTCCTCTTCCCCCCAAAACCCAAGGACACCCT
CATGATCTCCCGGACCCCCGAGGTCACATGCGTGGTGGTGGACGTGAGCCAC
GAAGACCCTGAGGTCAAGTTCAACTGGTACGTGGACGGCGTGGAGGTGCATA
ATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCC
GGGACGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCT
CCCTGTCTCCGGGTGGTGGTGGCGGATCAGGTGGGGGAGGCTCTGGTGGAGG
CGGTAGTGAGGTTCAGTTGGTGGAGTCCGGGGGAGGATTGGTGCAGGCTGGA
GGAAGTTTGAGGTTGTCCTGTGCCGCCAGCGGGGATGATTTGAGCTCCGTTGA
CATGGGATGGTACAGGCAGGCACCCGGAAAGGGACTGGAGTTCGTGGGAGT
GATTAGCAGGGGGGGAGGAAGCACATACTACGTGGACTCCGTGCAGGGAAG
GTTTACAATCTCCCGGGACAACGCTAAGAACACAGTGTACCTGCAGATGAAC
AGCCTCAGGGCCGAGGACACAGCAGTGTACTACTGCAAAACCGAGATTCCCT
ATTTTGACGATTACTGGGGCCAGGGCACACTGGTGACCGTTAGTTCC (SEQ ID
NO: 131)
GCTCAGACAAATTTCATGCCTATGGATGATTTGGAGCAGAGGCTG
TACGAACAGTTCATCCTGCAGCAGGGCCTGGAAGGTGGTGGCGGATCAGGTG
GGGGAGGCTCTGGTGGAGGCGGTAGTTCCGATACCGGTCGTCCTTTTGTCGAG
ATGTACTCCGAGATCCCCGAGATTATTCACATGACCGAGGGCCGCGAGCTGG
TGATTCCTTGTCGTGTGACCTCCCCTAACATCACCGTTACCCTGAAGAAGTTT
CCCCTGGATACACTGATCCCTGATGGCAAGAGGATTATCTGGGACTCCAGGA
AGGGATTCATTATTTCCAACGCCACCTACAAGGAGATCGGCCTCCTGACCTGC
GAAGCCACAGTTAACGGCCACCTCTACAAGACCAACTACCTGACACACCGGC
AGACCAACACCATCATCGACGTCGTGCTCAGCCCCTCCCACGGAATTGAGCT
GTCCGTCGGAGAAAAGCTGGTGCTGAACTGCACCGCCAGAACCGAACTGAAC
GTGGGCATCGACTTCAACTGGGAGTACCCCTCCTCCAAACACCAGCACAAGA
AACTCGTCAACAGGGACCTGAAAACTCAATCTGGCTCCGAAATGAAAAAGTT
CCTGTCCACCCTGACCATCGACGGCGTGACCAGGTCCGACCAGGGCCTGTAC
ACCTGCGCCGCCTCCTCCGGCCTGATGACCAAGAAGAACTCCACCTTCGTGAG
GGTGCACGAGAAGGACAAAACTCACACATGCCCACCGTGCCCAGCACCTGAA
CTCCTGGGGGGACCGTCAGTCTTCCTCTTCCCCCCAAAACCCAAGGACACCCT
CATGATCTCCCGGACCCCCGAGGTCACATGCGTGGTGGTGGACGTGAGCCAC
GAAGACCCTGAGGTCAAGTTCAACTGGTACGTGGACGGCGTGGAGGTGCATA
ATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCC
GGGACGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCT
CCCTGTCTCCGGGTGGTGGTGGCGGATCAGGTGGGGGAGGCTCTGGTGGAGG
CGGTAGTGAGGTTCAGTTGGTGGAGTCCGGGGGAGGATTGGTGCAGGCTGGA
GGAAGTTTGAGGTTGTCCTGTGCCGCCAGCGGGGGAGACTTGTCCTCCGTGG
ACATGGGATGGTACAGGCAGGCCCCTGGAAAGGGATTGGAGTTTGTGGGGGT
TATTTCCCGGACTGGAGGGTCCACCTATTATGTTGACAGTGTGCAGGGACGGT
TTACCATTTCTAGGGACAACGCTAAGAACACAGTTTACCTGCAGATGAATAG
CTTGAGGGCTGAGGACACTGCTGTGTATTATTGCAAGACCGAGATTCCAGGA
GGAGACGACTACTGGGGCCAGGGAACACTGGTGACCGTGAGTTCC (SEQ ID
NO: 132)
GAGGTTCAGTTGGTGGAGTCCGGGGGAGGATTGGTGCAGCCTGGA
GGAAGTTTGAGGTTGAGCTGTGCCGCATCCGGGTTGTACTTTAGCAGCTACGA
CATTGCATGGGTGAGGCAGGCACCTGGCAAGGGATTGGAGTTTGTGAGCGCC
ATCGACGTGAGCGGAGAGCATACATACTACGCTGACAGTGTGAAGGGCCGGT
TTACCATCTCCAGGGACAACGCTAAGAACACACTGTACCTGCAGATGAACAG
CCTGAGGGCAGAGGACACAGCAGTGTACTACTGCAACGCAGTGAAATGGAGC
GATTACGGACCAGACAACGACTACTGGGGACAGGGAACCCTCGTGACCGTGT
CTAGCGGTGGTGGCGGATCAGGTGGGGGAGGCTCTGGTGGAGGCGGTAGTTC
CGATACCGGTCGTCCTTTTGTCGAGATGTACTCCGAGATCCCCGAGATTATTC
ACATGACCGAGGGCCGCGAGCTGGTGATTCCTTGTCGTGTGACCTCCCCTAAC
ATCACCGTTACCCTGAAGAAGTTTCCCCTGGATACACTGATCCCTGATGGCAA
GAGGATTATCTGGGACTCCAGGAAGGGATTCATTATTTCCAACGCCACCTACA
AGGAGATCGGCCTCCTGACCTGCGAAGCCACAGTTAACGGCCACCTCTACAA
GACCAACTACCTGACACACCGGCAGACCAACACCATCATCGACGTCGTGCTC
AGCCCCTCCCACGGAATTGAGCTGTCCGTCGGAGAAAAGCTGGTGCTGAACT
GCACCGCCAGAACCGAACTGAACGTGGGCATCGACTTCAACTGGGAGTACCC
CTCCTCCAAACACCAGCACAAGAAACTCGTCAACAGGGACCTGAAAACTCAA
TCTGGCTCCGAAATGAAAAAGTTCCTGTCCACCCTGACCATCGACGGCGTGAC
CAGGTCCGACCAGGGCCTGTACACCTGCGCCGCCTCCTCCGGCCTGATGACCA
AGAAGAACTCCACCTTCGTGAGGGTGCACGAGAAGGACAAAACTCACACATG
CCCACCGTGCCCAGCACCTGAACTCCTGGGGGGACCGTCAGTCTTCCTCTTCC
CCCCAAAACCCAAGGACACCCTCATGATCTCCCGGACCCCCGAGGTCACATG
CGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCC
CCCATCGAGAAAACCATCTCCAAAGCCAAAGGGCAGCCCCGAGAACCACAG
GTGTACACCCTGCCCCCATCCCGGGACGAGCTGACCAAGAACCAGGTCAGCC
TGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTG
GCAGCAGGGGAACGTCTTCTCATGCTCCGTGATGCATGAGGCTCTGCACAAC
CACTACACGCAGAAGAGCCTCTCCCTGTCTCCGGGTGGTGGTGGCGGATCAG
GTGGGGGAGGCTCTGGTGGAGGCGGTAGTGAGGTTCAGTTGGTGGAGTCCGG
GGGAGGATTGGTGCAGGCTGGAGGAAGTTTGAGGTTGTCCTGTGCCGCCAGC
GGGGATGATTTGAGCTCCGTTGACATGGGATGGTACAGGCAGGCACCCGGAA
AGGGACTGGAGTTCGTGGGAGTGATTAGCAGGGGGGGAGGAAGCACATACT
ACGTGGACTCCGTGCAGGGAAGGTTTACAATCTCCCGGGACAACGCTAAGAA
CACAGTGTACCTGCAGATGAACAGCCTCAGGGCCGAGGACACAGCAGTGTAC
TACTGCAAAACCGAGATTCCCTATTTTGACGATTACTGGGGCCAGGGCACACT
GGTGACCGTTAGTTCC (SEQ ID NO: 133)
GAGGTTCAGTTGGTGGAGTCCGGGGGAGGATTGGTGCAGGCTGGA
GGAAGTTTGAGGTTGTCCTGTGCCGCCAGCGGGGATGATTTGAGCTCCGTTGA
CATGGGATGGTACAGGCAGGCACCCGGAAAGGGACTGGAGTTCGTGGGAGT
GATTAGCAGGGGGGGAGGAAGCACATACTACGTGGACTCCGTGCAGGGAAG
GTTTACAATCTCCCGGGACAACGCTAAGAACACAGTGTACCTGCAGATGAAC
AGCCTCAGGGCCGAGGACACAGCAGTGTACTACTGCAAAACCGAGATTCCCT
ATTTTGACGATTACTGGGGCCAGGGCACACTGGTGACCGTTAGTTCCGGTGGT
GGCGGATCAGGTGGGGGAGGCTCTGGTGGAGGCGGTAGTTCCGATACCGGTC
GTCCTTTTGTCGAGATGTACTCCGAGATCCCCGAGATTATTCACATGACCGAG
GGCCGCGAGCTGGTGATTCCTTGTCGTGTGACCTCCCCTAACATCACCGTTAC
CCTGAAGAAGTTTCCCCTGGATACACTGATCCCTGATGGCAAGAGGATTATCT
GGGACTCCAGGAAGGGATTCATTATTTCCAACGCCACCTACAAGGAGATCGG
CCTCCTGACCTGCGAAGCCACAGTTAACGGCCACCTCTACAAGACCAACTAC
CTGACACACCGGCAGACCAACACCATCATCGACGTCGTGCTCAGCCCCTCCC
ACGGAATTGAGCTGTCCGTCGGAGAAAAGCTGGTGCTGAACTGCACCGCCAG
AACCGAACTGAACGTGGGCATCGACTTCAACTGGGAGTACCCCTCCTCCAAA
CACCAGCACAAGAAACTCGTCAACAGGGACCTGAAAACTCAATCTGGCTCCG
AAATGAAAAAGTTCCTGTCCACCCTGACCATCGACGGCGTGACCAGGTCCGA
CCAGGGCCTGTACACCTGCGCCGCCTCCTCCGGCCTGATGACCAAGAAGAAC
TCCACCTTCGTGAGGGTGCACGAGAAGGACAAAACTCACACATGCCCACCGT
GCCCAGCACCTGAACTCCTGGGGGGACCGTCAGTCTTCCTCTTCCCCCCAAAA
CCCAAGGACACCCTCATGATCTCCCGGACCCCCGAGGTCACATGCGTGGTGG
TGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACACC
CTGCCCCCATCCCGGGACGAGCTGACCAAGAACCAGGTCAGCCTGACCTGCC
TGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACG
CAGAAGAGCCTCTCCCTGTCTCCGGGTGGTGGTGGCGGATCAGGTGGGGGAG
GCTCTGGTGGAGGCGGTAGTGAGGTTCAGTTGGTGGAGTCCGGGGGAGGATT
GGTGCAGCCTGGAGGAAGTTTGAGGTTGAGCTGTGCCGCATCCGGGTTGTAC
TTTAGCAGCTACGACATTGCATGGGTGAGGCAGGCACCTGGCAAGGGATTGG
AGTTTGTGAGCGCCATCGACGTGAGCGGAGAGCATACATACTACGCTGACAG
TGTGAAGGGCCGGTTTACCATCTCCAGGGACAACGCTAAGAACACACTGTAC
CTGCAGATGAACAGCCTGAGGGCAGAGGACACAGCAGTGTACTACTGCAACG
CAGTGAAATGGAGCGATTACGGACCAGACAACGACTACTGGGGACAGGGAA
CCCTCGTGACCGTGTCTAGC (SEQ ID NO: 134)
AGCGACCACGCCTGGAGC (SEQ ID NO: 135)
TACATCAGCTACAGCGGCATCACCACCTACAACCCCAGCCTGAAG
AGC (SEQ ID NO: 136)
GCCAGGACCACCGCCATGGACTAC (SEQ ID NO: 137)
TACACCAGCAGGCTGCACAGC (SEQ ID NO: 138)
AGGGCCAGCCAGGACATCAGCAGCTACCTGAAC (SEQ ID NO: 139)
CAGCAGGGCAACACCCTGCCCTACACC (SEQ ID NO: 140)

### REFERENCE TO AN ELECTRONIC SEQUENCE LISTING

The contents of the electronic sequence listing (076908-8004WO01.xml; Size: 242 KB; and Date of Creation: Sep. 7, 2023) is herein incorporated by reference in its entirety.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

1-35. (canceled)

36. An antibody fusion protein comprising an Ang-2-binding unit, an IL-6R-binding unit, and a VEGF-binding unit, wherein the VEGF-binding unit comprises a plurality of Ig-like domains of one or more VEGF receptors wherein the fusion protein is capable of binding substantially to Ang-2, IL-6R, and one or more VEGF family members.

37. The antibody fusion protein of claim 36; wherein the Ang-2-binding unit, the IL-6R-binding unit, and the VEGF-binding unit are linked together.

38. The antibody fusion protein of claim 36, wherein the Ang-2-binding unit comprises an antibody or a biologically active polypeptide that is capable of binding substantially to Ang-2.

39. The antibody fusion protein of claim 36, wherein the Ang-2 binding unit comprises SEQ ID NO: 6.

40. The antibody fusion protein of claim 36, wherein the IL-6R-binding unit comprises an antibody against IL-6R.

41. The antibody fusion protein of claim 40, wherein the antibody against IL-6R comprises a heavy chain and a light chain, wherein the heavy chain comprises the following complementarity determining regions (CDRs):
a. a CDR-H1 comprising the amino acid sequence of SEQ ID NO: 63,
b. a CDR-H2 comprising the amino acid sequence of SEQ ID NO: 64, and
c. a CDR-H3 comprising the amino acid sequence of SEQ ID NO: 65; and
the light chain comprises the following CDRs:
d. a CDR-L1 comprising the amino acid sequence of SEQ ID NO: 66,
e. a CDR-L2 comprising the amino acid sequence of SEQ ID NO: 67, and
f. a CDR-L3 comprising the amino acid sequence of SEQ ID NO: 68.

42. The antibody fusion protein of claim 41, wherein the heavy chain comprises SEQ ID NO: 4 and the light chain comprises SEQ ID NO: 5.

43. The antibody fusion protein of claim 36, wherein the VEGF-binding unit comprises VEGFR-1-D2 and VEGFR-2-D3 linked together.

44. The antibody fusion protein of claim 43, wherein VEGFR-1-D2 and VEGFR-2-D3 comprise SEQ ID NO: 1 and SEQ ID NO: 2, respectively.

45. The antibody fusion protein of claim 38, wherein the antibody that is capable of binding substantially to Ang-2 comprises an Ang-2 sdAb.

46. The antibody fusion protein of claim 40, wherein the antibody against IL-6R comprises an IL-6R sdAb.

47. The antibody fusion protein of claim 36, comprising: (a) an IL-6R antibody comprising a heavy chain and a light chain; (b) a VEGF-binding unit comprising VEGFR-1-D2 and VEGFR-2-D3, linked together in series; and (c) an Ang-2-binding polypeptide; wherein a C-terminus of the VEGF-binding unit is linked to an N-terminus of the light chain or the heavy chain of the IL-6R antibody; and the C-terminus of the heavy chain of the IL-6R antibody is linked to the N-terminus of the Ang-2-binding polypeptide.

48. The antibody fusion protein of claim 36, comprising: (a) an IL-6R antibody comprising a heavy chain and a light chain; (b) a VEGF-binding unit comprising VEGFR-1-D2 and VEGFR-2-D3, linked in series; and (c) an Ang-2 sdAb; wherein a C-terminus of the VEGF-binding unit is linked to an N-terminus of the light chain or the heavy chain of the IL-6R antibody; and the C-terminus of the heavy chain of the IL-6R antibody is linked to the N-terminus of the Ang-2 sdAb.

49. The antibody fusion protein of claim 36, comprising two fusion polypeptides, each of which comprises: (a) an IL-6R sdAb; (b) a VEGF-binding unit comprising VEGFR-1-D2 and VEGFR-2-D3, linked in series; (c) an Ang-2-binding polypeptide; and (d) an Fc domain of IgG1; wherein a C-terminus of the VEGF-binding unit is linked to an N-terminus of the Fc domain, the N-terminus of the VEGF-binding unit is linked to the C-terminus of the IL-6R sdAb, and the N-terminus of the Ang-2-binding polypeptide is linked to the C-terminus of the Fc domain.

50. The antibody fusion protein of claim 36, comprising two fusion polypeptides, each of which comprises: (a) IL-6R sdAb; (b) a VEGF-binding unit comprising VEGFR-1-D2 and VEGFR-2-D3, linked in series; (c) an Ang-2-binding polypeptide; and (d) an Fc domain of IgG1; wherein a C-terminus of the VEGF-binding unit is linked to an N-terminus of the Fc domain, the N-terminus of the VEGF-binding unit is linked to the C-terminus of the Ang-2 polypeptide, and the N-terminus of the IL-6R sdAb is linked to the C-terminus of the Fc domain.

51. The antibody fusion protein of claim 36, comprising two fusion polypeptides, each of which comprises: (a) an IL-6R sdAb; (b) a VEGF-binding unit comprising VEGFR-1-D2 and VEGFR-2-D3, linked in series; (c) an Ang-2 sdAb; and (d) an Fc domain of IgG1; wherein a C-terminus of the VEGF-binding unit is linked to an N-terminus of the Fc domain, the N-terminus of the VEGF-binding unit is linked to the C-terminus of the Ang-2 sdAb, and the N-terminus of the IL-6R sdAb is linked to the C-terminus of the Fc domain.

52. The antibody fusion protein of claim 36, comprising two fusion polypeptides, each of which comprises: (a) an IL-6R sdAb; (b) a VEGF-binding unit comprising VEGFR-1-D2 and VEGFR-2-D3, linked in series; (c) an Ang-2 sdAb; and (d) an Fc domain of IgG1; wherein a C-terminus of the VEGF-binding unit is linked to an N-terminus of the Fc domain, the N-terminus of the VEGF-binding unit is linked to the C-terminus of the IL-6R sdAb, and the N-terminus of the Ang-2 sdAb is linked to the C-terminus of the Fc domain.

53. The antibody fusion protein of claim 36, comprising polypeptides having a pair of amino acid sequences selected from the group consisting of SEQ ID NOs: 12 and 13, 14 and 15, 16 and 17, 18 and 19, 20 and 21, 22 and 23, 24 and 25, 26 and 27, 28 and 29, 38 and 39, and 40 and 41.

54. The antibody fusion protein of claim 43, comprising a polypeptide having an amino acid sequence selected from the group consisting of SEQ ID NOs: 57-62.

55. The antibody fusion protein of claim 36, wherein said antibody fusion protein is capable of binding to IL-6R, Ang-2, and at least a VEGF family member with an equilibrium dissociation constant (K_{D}) in the range from about 1×10-6 M to about 1×10-12 M or from about 1×10-8 M to about 1×10-12 M.

56. An isolated nucleic acid molecule encoding an antibody fusion protein of claim 36.

57. The isolated nucleic acid molecule of claim 56, wherein said nucleic acid molecule comprises a sequence selected from the group consisting of SEQ ID NOs: 78-134.

58. A method for treating or controlling at least a disease, condition, or disorder, in a subject in need thereof, which has etiology in aberrant angiogenesis or inflammation; wherein said method comprises administering to said subject an amount of a composition of an antibody fusion protein of claim 36.

59. The method of claim 58; wherein said disease, condition, or disorder is selected from the group consisting of: macular edema resulting from diabetes, uveitis, central and branch retinal vein occlusion, choroidal neovascularization, neovascular age-related macular degeneration, polypoidal choroidal vasculopathy, myopic choroidal neovascularization, vascular leak, non-proliferative and proliferative diabetic retinopathy, corneal neovascularization, corneal inflammation, myopic neovascularization, and neovascular glaucoma.

60. The method of claim 59; wherein the composition is administered to the subject as an eye drop, a punctal plug, intracameral, retrobulbar, subconjunctival, peribulbar, subtenon, juxta-scleral, trans-scleral, intravitreal, subretinal, or suprachoroidal injection.

61. A method for treating or controlling at least a systemic disease, condition, or disorder, in a subject in need thereof, which has etiology in aberrant angiogenesis or inflammation; wherein said method comprises administering to said subject an amount of a composition of an antibody fusion protein of claim 36.

62. The method of claim 61; wherein said systemic disease, condition, or disorder involves tumor growth, tumor metastasis, a combination of tumor growth and metastasis, atherosclerosis, and psoriasis.

63. A pharmaceutical composition for use to treat or control at least a disease, condition, or disorder, which has etiology in aberrant angiogenesis or inflammation; wherein said composition comprises an antibody fusion protein of claim 36.

64. The pharmaceutical composition of claim 63, wherein the disease, condition, or disorder is selected from the group consisting of macular edema resulting from diabetes, uveitis, central and branch retinal vein occlusion, choroidal neovascularization, neovascular age-related macular degeneration, polypoidal choroidal vasculopathy, myopic choroidal neovascularization, vascular leak, non-proliferative and proliferative diabetic retinopathy, corneal neovascularization, corneal inflammation, myopic neovascularization, neovascular glaucoma, tumor growth, tumor metastasis, a combination of tumor growth and metastasis, atherosclerosis, and psoriasis.

65. A pharmaceutical composition comprising an antibody fusion protein of claim 36, and a pharmaceutically acceptable carrier.


SEQUENCE LISTING (140 sequences)

SEQ ID NO: 1 - protein, 102 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTII

SEQ ID NO: 2 - protein, 103 aa, Homo sapiens
DVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 3 - protein, 231 aa, Homo sapiens
VEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 4 - protein, 447 aa, synthetic construct
  REGION 1..447: note = Tocilizumab heavy chain
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 5 - protein, 214 aa, synthetic construct [sequence omitted]

SEQ ID NO: 6 - protein, 26 aa, synthetic construct
  REGION 1..26: note = Artificial sequence
AQTNFMPMDDLEQRLYEQFILQQGLE

SEQ ID NO: 7 - protein, 28 aa, synthetic construct
  REGION 1..28: note = Artificial sequence
MGAQQKFQPLDELEQTLYEQFMLQQALE

SEQ ID NO: 8 - protein, 28 aa, synthetic construct
  REGION 1..28: note = Artificial sequence
MGAQQKYQPLDELDKTLYDQFMLQQGLE

SEQ ID NO: 9 - protein, 28 aa, synthetic construct
  REGION 1..28: note = Artificial sequence
MGAQHTFQPLDELEETLYYQWLYDQLLE

SEQ ID NO: 10 - protein, 18 aa, synthetic construct
  REGION 1..18: note = ARtificial sequence
AQQEECEWDPWTCEHMLE

SEQ ID NO: 11 - protein, 24 aa, synthetic construct
  REGION 1..24: note = Artificial sequence
AQTNIQEECEWDPWTCDHMPGKLE

SEQ ID NO: 12 - protein, 488 aa, synthetic construct
  REGION 1..488: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSAQTNFMPMDDLEQRLYEQFILQQGLE

SEQ ID NO: 13 - protein, 419 aa, synthetic construct
  REGION 1..419: note = Light chain of antibody fusion protein
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 483 aa, synthetic construct
  REGION 1..483: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSAQTNFMPMDDLEQRLYEQFILQQGLE

SEQ ID NO: 15 - protein, 419 aa, synthetic construct; identical to SEQ ID NO: 13
  REGION 1..419: note = Light chain of antibody fusion protein

SEQ ID NO: 16 - protein, 490 aa, synthetic construct
  REGION 1..490: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSMGAQQKFQPLDELEQTLYEQFMLQQALE

SEQ ID NO: 17 - protein, 419 aa, synthetic construct; identical to SEQ ID NO: 13
  REGION 1..419: note = Light chain of antibody fusion protein

SEQ ID NO: 18 - protein, 490 aa, synthetic construct
  REGION 1..490: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSMGAQQKYQPLDELDKTLYDQFMLQQGLE

SEQ ID NO: 19 - protein, 419 aa, synthetic construct; identical to SEQ ID NO: 13
  REGION 1..419: note = Light chain of antibody fusion protein

SEQ ID NO: 20 - protein, 485 aa, synthetic construct
  REGION 1..485: note = Heavy chain of anibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSMGAQQKYQPLDELDKTLYDQFMLQQGLE

SEQ ID NO: 21 - protein, 419 aa, synthetic construct; identical to SEQ ID NO: 13
  REGION 1..419: note = Light chain of antibody fusion protein

SEQ ID NO: 22 - protein, 490 aa, synthetic construct
  REGION 1..490: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSMGAQHTFQPLDELEETLYYQWLYDQLLE

SEQ ID NO: 23 - protein, 419 aa, synthetic construct; identical to SEQ ID NO: 13
  REGION 1..419: note = Light chain of antibody fusion protein

SEQ ID NO: 24 - protein, 480 aa, synthetic construct
  REGION 1..480: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSAQQEECEWDPWTCEHMLE

SEQ ID NO: 25 - protein, 419 aa, synthetic construct; identical to SEQ ID NO: 13
  REGION 1..419: note = Light chain of antibody fusion protein

SEQ ID NO: 26 - protein, 486 aa, synthetic construct
  REGION 1..486: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSAQTNIQEECEWDPWTCDHMPGKLE

SEQ ID NO: 27 - protein, 419 aa, synthetic construct; identical to SEQ ID NO: 13
  REGION 1..419: note = Light chain of antibody fusion protein

SEQ ID NO: 28 - protein, 481 aa, synthetic construct
  REGION 1..481: note = Heavy chain of antibody fusion protein
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSAQTNIQEECEWDPWTCDHMPGKLE

SEQ ID NO: 29 - protein, 419 aa, synthetic construct; identical to SEQ ID NO: 13
  REGION 1..419: note = Light chain of antibody fusion protein

SEQ ID NO: 30 - protein, 121 aa, synthetic construct
  REGION 1..121: note = sdAb A38-051
EVQLVESGGGLVQPGGSLRLSCAASGRTFSSYDFVWVRQAPGKGLEFVSAISVEGEHTYYADSVKGRFTISRDNAKNTLYLQMNSLRAEDTAVYYCNAVKWSDYGPDNDYWGQGTLVTVSS

SEQ ID NO: 31 - protein, 121 aa, synthetic construct
  REGION 1..121: note = sdAb A38-196
EVQLVESGGGLVQPGGSLRLSCAASGLYFSSYDIAWVRQAPGKGLEFVSAIDVSGEHTYYADSVKGRFTISRDNAKNTLYLQMNSLRAEDTAVYYCNAVKWSDYGPDNDYWGQGTLVTVSS

SEQ ID NO: 32 - protein, 128 aa, synthetic construct
  REGION 1..128: note = sdAB A41-089
EVQLQESGGGLVQPGGSLRLSCAASGFTLDAYAIGWFRQAPGKEREGVSTIGKSDGSTCYADSVKGRFTISRDNAKNTVWLQMNSLRAEDTAVYYCAARPWWVGDAPSIAAEYEYDLWGQGTQVTVSS

SEQ ID NO: 33 - protein, 128 aa, synthetic construct
  REGION 1..128: note = sdAb A41-093
EVQLQESGGGLVQPGGSLRLSCAASGFTLDEYAIGWFRQAPGKEREGVSCIGKADGSTCYADSVKGRFTISRDNAKNTVWLQMNSLRAEDTAVYYCAARPWWVGDPPSIGAEYEYDLWGQGTQVTVSS

SEQ ID NO: 34 - protein, 115 aa, synthetic construct
  REGION 1..115: note = sdAB A16-022
QVQLVESGGGLVQPGGSLRLSCAASGFRFSSYAMSWVRQAPGKGLEWVSKINSGGGITYYADSVKGRFTISRDNAKNTLYLQMNSLRAEDTAVYYCRDEGPFGSWGQGTQVTVSS

SEQ ID NO: 35 - protein, 115 aa, synthetic construct
  REGION 1..115: note = sdAb A16-030
QVQLVESGGGLVQPGGSLRLSCAASGFTFSSYAMSWVRQAPGKGLEWVSDINSGGSETYYADSVKGRFTISRDNAKNTLYLQMNSLRAEDTAVYYCRDKGPFGSWGQGTQVTVSS

SEQ ID NO: 36 - protein, 121 aa, synthetic construct
  REGION 1..121: note = sdAb A38-053
EVQLVESGGGLVQPGGSLRLSCAASGRTFSSYDIIWVRQAPGKGLEFVSAISTSGEHTYYADSVKGRFTISRDNAKNTLYLQMNSLRAEDTAVYYCNAVKWSDYGPQNDYWGQGTLVTVSS

SEQ ID NO: 37 - protein, 128 aa, synthetic construct
  REGION 1..128: note = sdAb A41-121
EVQLQESGGGLVQPGGSLRLSCAASGFTLDYYAIGWFRQAPGKEREGVSVIGKSDGSTCYADSVKGRFTISRDNAKNTVWLQMNSLRAEDTAVYYCAARPWWVGDRPSIAAEYEYDLWGQGTQVTVSS

SEQ ID NO: 38 - protein, 596 aa, synthetic construct
  REGION 1..596: note = Heavy chain of antibody fusion protein
MHSSALLCCLVLLTGVRAQVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSEVQLVESGGGLVQPGGSLRLSCAASGLYFSSYDIAWVRQAPGKGLEFVSAIDVSGEHTYYADSVKGRFTISRDNAKNTLYLQMNSLRAEDTAVYYCNAVKWSDYGPDNDYWGQGTLVTVSS

SEQ ID NO: 39 - protein, 437 aa, synthetic construct
  REGION 1..437: note = Light chain of antibody fusion protein
MHSSALLCCLVLLTGVRASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 603 aa, synthetic construct
  REGION 1..603: note = Heavy chain of antibody fusion protein
MHSSALLCCLVLLTGVRAQVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSEVQLQESGGGLVQPGGSLRLSCAASGFTLDAYAIGWFRQAPGKEREGVSTIGKSDGSTCYADSVKGRFTISRDNAKNTVWLQMNSLRAEDTAVYYCAARPWWVGDAPSIAAEYEYDLWGQGTQVTVSS

SEQ ID NO: 41 - protein, 437 aa, synthetic construct; identical to SEQ ID NO: 39
  REGION 1..437: note = Light chain of antibody fusion protein

SEQ ID NO: 42 - protein, 117 aa, synthetic construct
  REGION 1..117: note = IL-6R sdAb N_PM_119
EVQLVESGGGLVQAGGSLRLSCAASGDDLSSVDMGWYRQAPGKGLEFVGVISRGGGSTYYVDSVQGRFTISRDNAKNTVYLQMNSLRAEDTAVYYCKTEIPYFDDYWGQGTLVTVSS

SEQ ID NO: 43 - protein, 117 aa, synthetic construct
  REGION 1..117: note = IL-6R sdAb N_PM_123
EVQLVESGGGLVQAGGSLRLSCAASGESLSSVDMGWYRQAPGKGLEFVGVISRGGESTYYVDSVQGRFTISRDNAKNTVYLQMNSLRAEDTAVYYCKTEIPFGDDYWGQGTLVTVSS

SEQ ID NO: 44 - protein, 117 aa, synthetic construct
  REGION 1..117: note = IL-6R sdAb N_PM_180
EVQLVESGGGLVQAGGSLRLSCAASGGDLSSVDMGWYRQAPGKGLEFVGVISRTGGSTYYVDSVQGRFTISRDNAKNTVYLQMNSLRAEDTAVYYCKTEIPGGDDYWGQGTLVTVSS

SEQ ID NO: 45 - protein, 117 aa, synthetic construct
  REGION 1..117: note = IL-6R sdAb N_PM_184
EVQLVESGGGLVQAGGSLRLSCAASGESLSSVDMGWYRQAPGKGLEFVGVISRTGGSTYYVDSVQGRFTISRDNAKNTVYLQMNSLRAEDTAVYYCKTEIPGGDDVWGQGTLVTVSS

SEQ ID NO: 46 - protein, 117 aa, synthetic construct
  REGION 1..117: note = IL-6R sdAb H_PM_A5_108_008
EVQLVESGGGLVQPGGSLRLSCAASGGTFIDLDMGWYRQAPGNERQFVGVISRTGGSTYYADSVQGRFTISRDNAKNTLYLQMNSLRAEDTATYYCKTEIPGGDDYWGQGTLVTVSS

SEQ ID NO: 47 - protein, 117 aa, synthetic construct
  REGION 1..117: note = IL-6R sdAb H_PM_A5_108_031
EVQLVESGGGLVQPGGSLRLSCAASGGTFHLYDMGWYRQAPGNERQFVGVISRGGGSTYYADSVQGRFTISRDNAKNTLYLQMNSLRAEDTATYYCKTEIPGFQDYWGQGTLVTVSS

SEQ ID NO: 48 - protein, 117 aa, synthetic construct
  REGION 1..117: note = IL-6R sdAb H_PM_A5_108-040
EVQLVESGGGLVQPGGSLRLSCAASGPEFIDYDMGWYRQAPGNERQFVGVISRGGGSTYYADSVQGRFTISRDNAKNTLYLQMNSLRAEDTATYYCKTEIPFFDDYWGQGTLVTVSS

SEQ ID NO: 49 - protein, 117 aa, synthetic construct
  REGION 1..117: note = IL-6R sdAb H_PM_A5_108-_046
EVQLVESGGGLVQPGGSLRLSCAASGGTFIDLDMGWYRQAPGNERQFVGVISRGGGSVYYADSVQGRFTISRDNAKNTLYLQMNSLRAEDTATYYCKTEIPGGPDYWGQGTLVTVSS

SEQ ID NO: 50 - protein, 117 aa, synthetic construct
  REGION 1..117: note = IL-6R sdAb H_PM_A5_108_058
EVQLVESGGGLVQPGGSLRLSCAASGGTFIDYDMGWYRQAPGNERQFVGVISRGGGAVYYADSVQGRFTISRDNAKNTLYLQMNSLRAEDTATYYCKTEIPFFDDYWGQGTLVTVSS

SEQ ID NO: 51 - protein, 117 aa, synthetic construct
  REGION 1..117: note = IL-6R sdAb C82-111-Fc-011
QVQLVESGGGLVQPGGSLRLSCAASGPTFSNLDMGWYRQAPGKGLELVGVISRTGGSTYYADSVKGRFTISRDNAKNTLYLQMNSLRAEDTAVYYCKTEVPFSDDYWGQGTLVTVSS

SEQ ID NO: 52 - protein, 117 aa, synthetic construct
  REGION 1..117: note = IL-6R sdAb C82-111-Fc-013
QVQLVESGGGLVQPGGSLRLSCAASVPTFGDLDMGWYRQAPGKGLELVGVISRTGGSTYYADSVKGRFTISRDNAKNTLYLQMNSLRAEDTAVYYCKTEVPFGDDYWGQGTLVTVSS

SEQ ID NO: 53 - protein, 117 aa, synthetic construct
  REGION 1..117: note = IL-6R sdAb C82-111-Fc-020
QVQLVESGGGLVQPGGSLRLSCAASGDTFSNLDMGWYRQAPGKGLELVGVISRTGGSTYYADSVKGRFTISRDNAKNTLYLQMNSLRAEDTAVYYCKTEVPWADDYWGQGTLVTVSS

SEQ ID NO: 54 - protein, 117 aa, synthetic construct
  REGION 1..117: note = IL-6R sdAb C82-111-Fc-033
QVQLVESGGGLVQPGGSLRLSCAASVPTFSDVDMGWYRQAPGKGLELVGVISRTGGSTYYADSVKGRFTISRDNAKNTLYLQMNSLRAEDTAVYYCKTEVPFADDYWGQGTLVTVSS

SEQ ID NO: 55 - protein, 117 aa, synthetic construct
  REGION 1..117: note = IL-6R sdAb C82-111-Fc-043
QVQLVESGGGLVQPGGSLRLSCAASVPWFSNLDMGWYRQAPGKGLELVGVISRTGGMTYYADSVKGRFTISRDNAKNTLYLQMNSLRAEDTAVYYCKTEVPGSDDYWGQGTLVTVSS

SEQ ID NO: 56 - protein, 117 aa, synthetic construct
  REGION 1..117: note = IL-6R sdAb C82-111-Fc-059
QVQLVESGGGLVQPGGSLRLSCAASGATFSNLDMGWYRQAPGKGLELVGVISRTGGSTYYADSVKGRFTISRDNAKNTLYLQMNSLRAEDTAVYYCKTEVPFDDDYWGQGTLVTVSS

SEQ ID NO: 57 - protein, 624 aa, synthetic construct
  REGION 1..624: note = EB-105BId1 protein
MHSSALLCCLVLLTGVRAEVQLVESGGGLVQAGGSLRLSCAASGDDLSSVDMGWYRQAPGKGLEFVGVISRGGGSTYYVDSVQGRFTISRDNAKNTVYLQMNSLRAEDTAVYYCKTEIPYFDDYWGQGTLVTVSSGGSGGGSGGGGSGGGGGSGGGGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSAQTNFMPMDDLEQRLYEQFILQQGLE

SEQ ID NO: 58 - protein, 624 aa, synthetic construct
  REGION 1..624: note = EB-105BId3 protein
MHSSALLCCLVLLTGVRAEVQLVESGGGLVQAGGSLRLSCAASGGDLSSVDMGWYRQAPGKGLEFVGVISRTGGSTYYVDSVQGRFTISRDNAKNTVYLQMNSLRAEDTAVYYCKTEIPGGDDYWGQGTLVTVSSGGSGGGSGGGGSGGGGGSGGGGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSAQTNFMPMDDLEQRLYEQFILQQGLE

SEQ ID NO: 59 - protein, 622 aa, synthetic construct
  REGION 1..622: note = EB-105BId2 protein
MHSSALLCCLVLLTGVRAAQTNFMPMDDLEQRLYEQFILQQGLEGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLVESGGGLVQAGGSLRLSCAASGDDLSSVDMGWYRQAPGKGLEFVGVISRGGGSTYYVDSVQGRFTISRDNAKNTVYLQMNSLRAEDTAVYYCKTEIPYFDDYWGQGTLVTVSS

SEQ ID NO: 60 - protein, 622 aa, synthetic construct
  REGION 1..622: note = EB-105BId4 protein
MHSSALLCCLVLLTGVRAAQTNFMPMDDLEQRLYEQFILQQGLEGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLVESGGGLVQAGGSLRLSCAASGGDLSSVDMGWYRQAPGKGLEFVGVISRTGGSTYYVDSVQGRFTISRDNAKNTVYLQMNSLRAEDTAVYYCKTEIPGGDDYWGQGTLVTVSS

SEQ ID NO: 61 - protein, 717 aa, synthetic construct
  REGION 1..717: note = EB-105BIe1 protein
MHSSALLCCLVLLTGVRAEVQLVESGGGLVQPGGSLRLSCAASGLYFSSYDIAWVRQAPGKGLEFVSAIDVSGEHTYYADSVKGRFTISRDNAKNTLYLQMNSLRAEDTAVYYCNAVKWSDYGPDNDYWGQGTLVTVSSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLVESGGGLVQAGGSLRLSCAASGDDLSSVDMGWYRQAPGKGLEFVGVISRGGGSTYYVDSVQGRFTISRDNAKNTVYLQMNSLRAEDTAVYYCKTEIPYFDDYWGQGTLVTVSS

SEQ ID NO: 62 - protein, 717 aa, synthetic construct
  REGION 1..717: note = EB-105BIe2 protein
MHSSALLCCLVLLTGVRAEVQLVESGGGLVQAGGSLRLSCAASGDDLSSVDMGWYRQAPGKGLEFVGVISRGGGSTYYVDSVQGRFTISRDNAKNTVYLQMNSLRAEDTAVYYCKTEIPYFDDYWGQGTLVTVSSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLVESGGGLVQPGGSLRLSCAASGLYFSSYDIAWVRQAPGKGLEFVSAIDVSGEHTYYADSVKGRFTISRDNAKNTLYLQMNSLRAEDTAVYYCNAVKWSDYGPDNDYWGQGTLVTVSS

SEQ ID NO: 63 - protein, 6 aa, synthetic construct
  REGION 1..6: note = CDR1 of Tocilizumab HC
SDHAWS

SEQ ID NO: 64 - protein, 16 aa, synthetic construct
  REGION 1..16: note = CDR2 of Tocilizumab HC
YISYSGITTYNPSLKS

SEQ ID NO: 65 - protein, 8 aa, synthetic construct
  REGION 1..8: note = CDR3 of Tocilizumab HC
ARTTAMDY

SEQ ID NO: 66 - protein, 11 aa, synthetic construct
  REGION 1..11: note = CDR1 of Tocilizumab LC
RASQDISSYLN

SEQ ID NO: 67 - protein, 7 aa, synthetic construct
  REGION 1..7: note = CDR2 of Tocilizumab LC
YTSRLHS

SEQ ID NO: 68 - protein, 9 aa, synthetic construct
  REGION 1..9: note = CDR3 of Tocilizumab LC
QQGNTLPYT

SEQ ID NO: 69 - protein, 446 aa, synthetic construct
  REGION 1..446: note = Sarilumab HC protein
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTYLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 214 aa, synthetic construct
  REGION 1..214: note = Sarilumab LC protein
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 443 aa, synthetic construct
  REGION 1..443: note = Satralizumab HC protein
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHAHYTQKSLSLSP

SEQ ID NO: 72 - protein, 214 aa, synthetic construct
  REGION 1..214: note = Satralizumab LC protein
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 306 bp, Homo sapiens
  source 1..306: mol_type = genomic DNA
agcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatc

SEQ ID NO: 74 - DNA, 309 bp, Homo sapiens
  source 1..309: mol_type = genomic DNA
gacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaag

SEQ ID NO: 75 - DNA, 693 bp, Homo sapiens
  source 1..693: mol_type = genomic DNA
gttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccg

SEQ ID NO: 76 - DNA, 1341 bp, synthetic construct
  misc_feature 1..1341: note = DNA sequence of Tocilizumab HC
  source 1..1341: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccg

SEQ ID NO: 77 - DNA, 652 bp, synthetic construct
  misc_feature 1..652: note = DNA sequence of Tolcilizumab LC
  source 1..652: mol_type = other DNA
gacattcagatgacccagagccccagcagcctgagcgccagcgtgggagacagagtgaccatcacctgcagagccagccaggacatctccagctacctgaactggtatcagcagaaacccggcaaagccccaaaactgctgatctactacaccagtagactgcacagcggcgtgcccagcagattctcaggaagcggctccggaaccgacttcaccttcactatcagcagcctgcagcccgaagatattgctacttactactgccagcaggggaacaccctgccctataccttcggccagggcaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttgattctaga

SEQ ID NO: 78 - DNA, 75 bp, synthetic construct
  misc_feature 1..75: note = DNA sequence of Ang-2-binding Peptide 1
  source 1..75: mol_type = other DNA
gctcagaccaattttatgcctatggatgatctggaacagagactgtacgaacagttcatcctccaacagggactg

SEQ ID NO: 79 - DNA, 84 bp, synthetic construct
  misc_feature 1..84: note = DNA sequence of Ang-2-binding Peptide 2
  source 1..84: mol_type = other DNA
atgggcgctcagcagaagttccagcccctggacgagctggagcagaccctgtacgagcagttcatgctccagcaggccctggag

SEQ ID NO: 80 - DNA, 84 bp, synthetic construct
  misc_feature 1..84: note = DNA sequence of Ang-2-binding Peptide 3
  source 1..84: mol_type = other DNA
atgggcgctcagcagaagtaccagcccctggacgagctggacaagaccctgtacgaccagttcatgctccagcaggggctggaa

SEQ ID NO: 81 - DNA, 84 bp, synthetic construct
  misc_feature 1..84: note = DNA sequence of Ang-2-binding Peptide 4
  source 1..84: mol_type = other DNA
atgggcgctcagcacaccttccagcccctggacgagctggaggagacactgtactaccagtggctctacgaccagctgctggag

SEQ ID NO: 82 - DNA, 54 bp, synthetic construct
  misc_feature 1..54: note = DNA sequence of Ang-2-binding Peptide 5
  source 1..54: mol_type = other DNA
gctcagcaagaagagtgtgaatgggacccttggacctgtgaacacatgctggaa

SEQ ID NO: 83 - DNA, 72 bp, synthetic construct
  misc_feature 1..72: note = DNA sequence of Ang-2-binding Peptide 6
  source 1..72: mol_type = other DNA
gctcagaccaatattcaagaagaatgtgaatgggacccttggacctgtgatcacatgcccggcaaactggaa

SEQ ID NO: 84 - DNA, 1464 bp, synthetic construct
  misc_feature 1..1464: note = DNA sequence of EB-105BI-7CA heavy chain
  source 1..1464: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggatcaggtgggggaggctctggtggaggcggtagtgctcagaccaattttatgcctatggatgatctggaacagagactgtacgaacagttcatcctccaacagggactggaa

SEQ ID NO: 85 - DNA, 1257 bp, synthetic construct
  misc_feature 1..1257: note = DNA sequence of EB-105BI-7CA light chain
  source 1..1257: mol_type = other DNA
agcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacattcagatgacccagagccccagcagcctgagcgccagcgtgggagacagagtgaccatcacctgcagagccagccaggacatctccagctacctgaactggtatcagcagaaacccggcaaagccccaaaactgctgatctactacaccagtagactgcacagcggcgtgcccagcagattctcaggaagcggctccggaaccgacttcaccttcactatcagcagcctgcagcccgaagatattgctacttactactgccagcaggggaacaccctgccctataccttcggccagggcaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 86 - DNA, 1449 bp, synthetic construct
  misc_feature 1..1449: note = DNA sequence of EB-105BI-7CB heavy chain
  source 1..1449: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggttctggaggtggtggatcagctcagaccaattttatgcctatggatgatctggaacagagactgtacgaacagttcatcctccaacagggactggaa

SEQ ID NO: 87 - DNA, 1257 bp, synthetic construct; identical to SEQ ID NO: 85
  misc_feature 1..1257: note = DNA sequence of EB-105BI-7CB light chain
  source 1..1257: mol_type = other DNA

SEQ ID NO: 88 - DNA, 1470 bp, synthetic construct
  misc_feature 1..1470: note = DNA sequence of EB-105BI-10c heavy chain
  source 1..1470: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggatcaggtgggggaggctctggtggaggcggtagtatgggcgctcagcagaagttccagcccctggacgagctggagcagaccctgtacgagcagttcatgctccagcaggccctggag

SEQ ID NO: 89 - DNA, 1257 bp, synthetic construct; identical to SEQ ID NO: 85
  misc_feature 1..1257: note = DNA sequence of EB-105BI-10C light chain
  source 1..1257: mol_type = other DNA

SEQ ID NO: 90 - DNA, 1470 bp, synthetic construct
  misc_feature 1..1470: note = DNA sequence of EB-105-BI-15CA heavy chain
  source 1..1470: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggatcaggtgggggaggctctggtggaggcggtagtatgggcgctcagcagaagtaccagcccctggacgagctggacaagaccctgtacgaccagttcatgctccagcaggggctggaa

SEQ ID NO: 91 - DNA, 1257 bp, synthetic construct; identical to SEQ ID NO: 85
  misc_feature 1..1257: note = DNA sequence of EB-105BI-15CA light chain
  source 1..1257: mol_type = other DNA

SEQ ID NO: 92 - DNA, 1455 bp, synthetic construct
  misc_feature 1..1455: note = DNA sequence of EB-105BI-15CB heavy chain
  source 1..1455: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggttctggaggtggtggatcaatgggcgctcagcagaagtaccagcccctggacgagctggacaagaccctgtacgaccagttcatgctccagcaggggctggaa

SEQ ID NO: 93 - DNA, 1257 bp, synthetic construct; identical to SEQ ID NO: 85
  misc_feature 1..1257: note = DNA sequence of EB-105BI-15CB light chain
  source 1..1257: mol_type = other DNA

SEQ ID NO: 94 - DNA, 1470 bp, synthetic construct
  misc_feature 1..1470: note = DNA sequence of EB-105BI-21C heavy chain
  source 1..1470: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggatcaggtgggggaggctctggtggaggcggtagtatgggcgctcagcacaccttccagcccctggacgagctggaggagacactgtactaccagtggctctacgaccagctgctggag

SEQ ID NO: 95 - DNA, 1257 bp, synthetic construct; identical to SEQ ID NO: 85
  misc_feature 1..1257: note = DNA sequence of EB-105BI-21C light chain
  source 1..1257: mol_type = other DNA

SEQ ID NO: 96 - DNA, 1440 bp, synthetic construct
  misc_feature 1..1440: note = DNA sequence of EB-105BI-Con4C heavy chain
  source 1..1440: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggatcaggtgggggaggctctggtggaggcggtagtgctcagcaagaagagtgtgaatgggacccttggacctgtgaacacatgctggaa

SEQ ID NO: 97 - DNA, 1257 bp, synthetic construct; identical to SEQ ID NO: 85
  misc_feature 1..1257: note = DNA sequence of EB-105BI-Con4C light chain
  source 1..1257: mol_type = other DNA

SEQ ID NO: 98 - DNA, 1458 bp, synthetic construct
  misc_feature 1..1458: note = DNA sequence of EB-105BI-Con4-40CA heavy chain
  source 1..1458: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggatcaggtgggggaggctctggtggaggcggtagtgctcagaccaatattcaagaagaatgtgaatgggacccttggacctgtgatcacatgcccggcaaactggaa

SEQ ID NO: 99 - DNA, 1257 bp, synthetic construct; identical to SEQ ID NO: 85
  misc_feature 1..1257: note = DNA sequence of EB-105BI-Con4-40CA light chain
  source 1..1257: mol_type = other DNA

SEQ ID NO: 100 - DNA, 1443 bp, synthetic construct
  misc_feature 1..1443: note = DNA sequence of EB-105BI-Con4-40CB heavy chain
  source 1..1443: mol_type = other DNA
caggtgcagctgcaggagagcggccccggactggtgaggccttcccagaccctgagcctgacatgcacagtgagcggctacagcatcacaagcgaccacgcctggagctgggtgagacagccccccggaagaggcctggaatggatcggatatatctcctactcaggcattaccacctacaacccctctctgaaaagcagagtgaccatgctgagagatactagcaagaaccagtttagcctgagactgtctagcgtgactgccgccgacaccgccgtgtactactgcgccagatccctggcccggaccacagccatggattactggggccagggaagcctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggttctggaggtggtggatcagctcagaccaatattcaagaagaatgtgaatgggacccttggacctgtgatcacatgcccggcaaactggaa

SEQ ID NO: 101 - DNA, 1257 bp, synthetic construct; identical to SEQ ID NO: 85
  misc_feature 1..1257: note = DNA sequence of EB-105BI-Con4-40CB light chain
  source 1..1257: mol_type = other DNA

SEQ ID NO: 102 - DNA, 363 bp, synthetic construct
  misc_feature 1..363: note = DNA sequence of Ang-2 sdAb A38-051
  source 1..363: mol_type = other DNA
gaggtgcagctggtggagagcggcggcggactggtgcagcctggaggatctctgaggctgtcctgcgccgcttctggccggaccttctccagctacgactttgtttgggtgcggcaggcccctggcaagggactggagttcgtgtccgccatctccgttgagggcgagcacacctactatgctgatagcgtgaagggccggttcaccatcagcagggataacgccaagaataccctgtatctgcagatgaatagcctgcgggctgaggacaccgccgtgtattactgtaacgccgtgaagtggagcgactacggccccgacaacgattactggggccagggcaccctggtgaccgtgtcctcc

SEQ ID NO: 103 - DNA, 363 bp, synthetic construct
  misc_feature 1..363: note = DNA sequence of Ang-2 sdAb A38-196
  source 1..363: mol_type = other DNA
gaggtgcagctggtggagagcggcggcggactggtgcagcctggaggatctctgaggctgtcctgcgccgcttctggcctttatttctccagctacgacatcgcttgggtgcggcaggcccctggcaagggactggagttcgtgtccgccatcgatgtgtccggcgagcacacctactatgctgatagcgtgaagggccggttcaccatcagcagggataacgccaagaataccctgtatctgcagatgaatagcctgcgggctgaggacaccgccgtgtattactgtaacgccgtgaagtggagcgactacggccccgacaacgattactggggccagggcaccctggtgaccgtgtcctcc

SEQ ID NO: 104 - DNA, 384 bp, synthetic construct
  misc_feature 1..384: note = DNA sequence of Ang-2 sdAb A41-089
  source 1..384: mol_type = other DNA
gaggtgcagctgcaggagagcggcggcggactggtgcagccaggaggaagcctgcggctgtcctgtgccgcttccggctttaccctggatgcgtatgctatcggctggtttaggcaggcccctggcaaggagagggagggcgtgtccacgatcggcaagtccgacggcagcacctgttacgccgacagcgtgaagggcaggttcaccatcagcagggacaacgctaagaataccgtgtggctgcagatgaactccctgcgggctgaggacaccgccgtgtactattgcgccgctcggccctggtgggtgggcgacgcgccttctatcgctgccgagtacgagtacgatctgtggggccagggcacccaggtgaccgtgagctcc

SEQ ID NO: 105 - DNA, 384 bp, synthetic construct
  misc_feature 1..384: note = DNA sequence of ANg-2 sdAb A41-093
  source 1..384: mol_type = other DNA
gaggtgcagctgcaggagagcggcggcggactggtgcagccaggaggaagcctgcggctgtcctgtgccgcttctggctttaccctggatgagtatgctatcggctggtttaggcaggcccctggcaaggagagggagggcgtgtcctgtatcggcaaggcggacggcagcacctgttacgccgacagcgtgaagggcaggttcaccatcagcagggacaacgctaagaataccgtgtggctgcagatgaactccctgcgggctgaggacaccgccgtgtactattgcgccgctcggccctggtgggtgggcgaccctccttctatcggtgccgagtacgagtacgatctgtggggccagggcacccaggtgaccgtgagctcc

SEQ ID NO: 106 - DNA, 345 bp, synthetic construct
  misc_feature 1..345: note = DNA sequence of Ang-2 sdAb A16-022
  source 1..345: mol_type = other DNA
caggtgcagctggtggagagcggcggcggactggtgcagcctggaggaagcctgcggctgtcctgtgccgccagcggattcaggtttagcagctacgccatgtcctgggtgaggcaggctcctggcaagggcctggagtgggtgagcaagatcaatagcggcggcggcatcacctactacgctgactccgtgaagggcaggtttaccatctccagggataacgccaagaataccctgtacctgcagatgaatagcctgcgggctgaggacaccgccgtgtattactgtcgggacgagggcccttttggcagctggggccagggcacccaggtgacagtgagctcc

SEQ ID NO: 107 - DNA, 345 bp, synthetic construct
  misc_feature 1..345: note = DNA sequence of Ang-2 sdAb A16-030
  source 1..345: mol_type = other DNA
caggtgcagctggtggagagcggcggcggactggtgcagcctggaggaagcctgcggctgtcctgtgccgccagcggattcacctttagcagctacgccatgtcctgggtgaggcaggctcctggcaagggcctggagtgggtgagcgacatcaatagcggcggcagtgagacctactacgctgactccgtgaagggcaggtttaccatctccagggataacgccaagaataccctgtacctgcagatgaatagcctgcgggctgaggacaccgccgtgtattactgtcgggacaagggcccttttggcagctggggccagggcacccaggtgacagtgagctcc

SEQ ID NO: 108 - DNA, 363 bp, synthetic construct
  misc_feature 1..363: note = DNA sequence of Ang-2 sdAb A38-053
  source 1..363: mol_type = other DNA
gaggtgcagctggtggagagcggcggcggactggtgcagcctggaggatctctgaggctgtcctgcgccgcttctggccggaccttctccagctacgacatcatttgggtgcggcaggcccctggcaagggactggagttcgtgtccgccatctccacgtccggcgagcacacctactatgctgatagcgtgaagggccggttcaccatcagcagggataacgccaagaataccctgtatctgcagatgaatagcctgcgggctgaggacaccgccgtgtattactgtaacgccgtgaagtggagcgactacggcccccagaacgattactggggccagggcaccctggtgaccgtgtcctcc

SEQ ID NO: 109 - DNA, 384 bp, synthetic construct
  misc_feature 1..384: note = DNA sequence of Ang-2 sdAb A41-121
  source 1..384: mol_type = other DNA
gaggtgcagctgcaggagagcggcggcggactggtgcagccaggaggaagcctgcggctgtcctgtgccgcttccggctttaccctggattactatgctatcggctggtttaggcaggcccctggcaaggagagggagggcgtgtccgtgatcggcaagtccgacggcagcacctgttacgccgacagcgtgaagggcaggttcaccatcagcagggacaacgctaagaataccgtgtggctgcagatgaactccctgcgggctgaggacaccgccgtgtactattgcgccgctcggccctggtgggtgggcgacaggccttctatcgctgccgagtacgagtacgatctgtggggccagggcacccaggtgaccgtgagctcc

SEQ ID NO: 110 - DNA, 1734 bp, synthetic construct
  misc_feature 1..1734: note = DNA sequence of EB-105BIc1 heavy chain
  source 1..1734: mol_type = other DNA
caggttcagctccaggagtcaggcccaggactcgttaggccatcccagacactctccctcacatgcacagtctccggctactccatcacatcagaccacgcctggtcctgggtcaggcagcctcctggaagaggactcgaatggatcggatatatctcctactccggcattaccacctacaacccctcactgaagtcccgcgtgaccatgttgagagatactagcaagaaccagttctccctgagactgagctccgtgactgccgctgacaccgctgtttactattgcgccagatccctggcccggaccacagctatggattactggggccagggaagcctggtgaccgtttcttccgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggaagagatgactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcggaggaggaagtggaggaggaggaagcgaggttcagttggtggagtccgggggaggattggtgcagcctggaggaagtttgaggttgagctgtgccgcatccgggttgtactttagcagctacgacattgcatgggtgaggcaggcacctggcaagggattggagtttgtgagcgccatcgacgtgagcggagagcatacatactacgctgacagtgtgaagggccggtttaccatctccagggacaacgctaagaacacactgtacctgcagatgaacagcctgagggcagaggacacagcagtgtactactgcaacgcagtgaaatggagcgattacggaccagacaacgactactggggacagggaaccctcgtgaccgtgtctagc

SEQ ID NO: 111 - DNA, 1257 bp, synthetic construct; identical to SEQ ID NO: 85
  misc_feature 1..1257: note = DNA sequence of EB-105BIc1 light chain
  source 1..1257: mol_type = other DNA

SEQ ID NO: 112 - DNA, 1755 bp, synthetic construct
  misc_feature 1..1755: note = DNA sequence of EB-105BIc2 heavy chain
  source 1..1755: mol_type = other DNA
caggttcagctccaggagtcaggcccaggactcgttaggccatcccagacactctccctcacatgcacagtctccggctactccatcacatcagaccacgcctggtcctgggtcaggcagcctcctggaagaggactcgaatggatcggatatatctcctactccggcattaccacctacaacccctcactgaagtcccgcgtgaccatgttgagagatactagcaagaaccagttctccctgagactgagctccgtgactgccgctgacaccgctgtttactattgcgccagatccctggcccggaccacagctatggattactggggccagggaagcctggtgaccgtttcttccgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggaagagatgactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcggaggaggaagtggaggaggaggaagcgaggttcagttgcaggagagcgggggaggattggtgcagcctggaggaagtttgaggttgagctgtgccgcatccgggtttacattggacgcatacgctattggatggttcaggcaggcacctggcaaggagagggagggagttagcactatcggcaagagtgacggcagcacatgctacgccgactccgttaagggcaggtttaccatttcccgggacaacgccaagaacaccgtgtggctgcagatgaacagcctgagggccgaggacacagcagtgtattattgcgccgccaggccctggtgggttggagacgctccttctatcgccgccgagtacgagtacgacctgtggggacagggcacccaggttactgtgtccagc

SEQ ID NO: 113 - DNA, 1257 bp, synthetic construct; identical to SEQ ID NO: 85
  misc_feature 1..1257: note = DNA sequence of EB-105BIc2 light chain
  source 1..1257: mol_type = other DNA

SEQ ID NO: 114 - DNA, 351 bp, synthetic construct
  misc_feature 1..351: note = DNA sequence of IL-6R sdAb N_PM_119
  source 1..351: mol_type = other DNA
gaggtgcagttggtggagtctgggggaggattggtgcaggctgggggctcactgagactctcctgtgcagcctctggagatgatttaagtagcgtagacatgggctggtaccgccaggctccagggaagggcctggagtttgtcggcgttattagccgtggtggtggtagcacatactatgtagactccgtgcagggccgattcaccatctccagagacaacgccaagaacacggtgtatctgcaaatgaacagcctgcgggccgaggacacggccgtgtattactgtaagacagaaattccatattttgatgactactggggccaggggaccctggtcaccgtctcctca

SEQ ID NO: 115 - DNA, 351 bp, synthetic construct
  misc_feature 1..351: note = DNA sequence of IL-6R sdAb N_PM_123
  source 1..351: mol_type = other DNA
gaggtgcagttggtggagtctgggggaggattggtgcaggctgggggctcactgagactctcctgtgcagcctctggagagtccttaagtagcgtagacatgggctggtaccgccaggctccagggaagggcctggagtttgtcggcgttattagccgtggtggtgagagcacatactatgtagactccgtgcagggccgattcaccatctccagagacaacgccaagaacacggtgtatctgcaaatgaacagcctgcgggccgaggacacggccgtgtattactgtaagacagaaattccatttggggatgactactggggccaggggaccctggtcaccgtctcctca

SEQ ID NO: 116 - DNA, 351 bp, synthetic construct
  misc_feature 1..351: note = DNA sequence of IL-6R sdAb N_PM_180
  source 1..351: mol_type = other DNA
gaggtgcagttggtggagtctgggggaggattggtgcaggctgggggctcactgagactctcctgtgcagcctctggaggtgatttaagtagcgtagacatgggctggtaccgccaggctccagggaagggcctggagtttgtcggcgttattagcaggacgggtggtagcacatactatgtagactccgtgcagggccgattcaccatctccagagacaacgccaagaacacggtgtatctgcaaatgaacagcctgcgggccgaggacacggccgtgtattactgtaagacagaaattccagggggggatgactactggggccaggggaccctggtcaccgtctcctca

SEQ ID NO: 117 - DNA, 351 bp, synthetic construct
  misc_feature 1..351: note = DNA sequence of IL-6R sdAb N_PM_184
  source 1..351: mol_type = other DNA
gaggtgcagttggtggagtctgggggaggattggtgcaggctgggggctcactgagactctcctgtgcagcctctggagagtccttaagtagcgtagacatgggctggtaccgccaggctccagggaagggcctggagtttgtcggcgttattagccgtacgggtggtagcacatactatgtagactccgtgcagggccgattcaccatctccagagacaacgccaagaacacggtgtatctgcaaatgaacagcctgcgggccgaggacacggccgtgtattactgtaagacagaaattccagggggggatgacgtttggggccaggggaccctggtcaccgtctcctca

SEQ ID NO: 118 - DNA, 351 bp, synthetic construct
  misc_feature 1..351: note = DNA sequence of IL-6R sdAb H_PM_A5_108_008
  source 1..351: mol_type = other DNA
gaggtgcagttggtggagagcgggggggggctggtgcagcctggaggaagtttgaggttgagctgtgccgcaagcggggggacattcattgatttggacatgggatggtacaggcaggcacctggaaacgagaggcagtttgtgggggtgatttcccggactggaggatccacatactacgctgacagcgtgcaggggaggttcaccatctcccgggacaacgccaagaacaccctgtacctgcagatgaactccctgagggctgaggacactgctacctattactgcaagaccgagattcccggaggagacgactactggggccagggcacactggtgaccgtgagctcc

SEQ ID NO: 119 - DNA, 351 bp, synthetic construct
  misc_feature 1..351: note = DNA sequence of IL-6R sdAb H_PM_A5_108_031
  source 1..351: mol_type = other DNA
gaggtgcagttggtggagagcgggggggggctggtgcagcctggaggaagtttgaggttgagctgtgccgcaagcggggggacattccatctgtacgacatgggatggtacaggcaggcacctggaaacgagaggcagtttgtgggggtgatttcccgcggcggaggatccacatactacgctgacagcgtgcaggggaggttcaccatctcccgggacaacgccaagaacaccctgtacctgcagatgaactccctgagggctgaggacactgctacctattactgcaagaccgagattcccggatttcaggactactggggccagggcacactggtgaccgtgagctcc

SEQ ID NO: 120 - DNA, 351 bp, synthetic construct
  misc_feature 1..351: note = DNA sequence of IL-6R sdAb H_PM_A5_108_040
  source 1..351: mol_type = other DNA
gaggtgcagttggtggagagcgggggggggctggtgcagcctggaggaagtttgaggttgagctgtgccgcaagcgggccggagttcattgattacgacatgggatggtacaggcaggcacctggaaacgagaggcagtttgtgggggtgatttcccgcggcggaggatccacatactacgctgacagcgtgcaggggaggttcaccatctcccgggacaacgccaagaacaccctgtacctgcagatgaactccctgagggctgaggacactgctacctattactgcaagaccgagattcccttttttgacgactactggggccagggcacactggtgaccgtgagctcc

SEQ ID NO: 121 - DNA, 351 bp, synthetic construct
  misc_feature 1..351: note = DNA sequence of IL-6R sdAb H_PM_A5_108_046
  source 1..351: mol_type = other DNA
gaggtgcagttggtggagagcgggggggggctggtgcagcctggaggaagtttgaggttgagctgtgccgcaagcggggggacattcattgatctggacatgggatggtacaggcaggcacctggaaacgagaggcagtttgtgggggtgatttcccgcggcggaggatccgtttactacgctgacagcgtgcaggggaggttcaccatctcccgggacaacgccaagaacaccctgtacctgcagatgaactccctgagggctgaggacactgctacctattactgcaagaccgagattcccggaggacctgactactggggccagggcacactggtgaccgtgagctcc

SEQ ID NO: 122 - DNA, 351 bp, synthetic construct
  misc_feature 1..351: note = DNA sequence of IL-6R sdAb H_PM_A5_108_058
  source 1..351: mol_type = other DNA
gaggtgcagttggtggagagcgggggggggctggtgcagcctggaggaagtttgaggttgagctgtgccgcaagcggggggacattcattgattacgacatgggatggtacaggcaggcacctggaaacgagaggcagtttgtgggggtgatttcccgcggcggaggagctgtttactacgctgacagcgtgcaggggaggttcaccatctcccgggacaacgccaagaacaccctgtacctgcagatgaactccctgagggctgaggacactgctacctattactgcaagaccgagattcccttttttgacgactactggggccagggcacactggtgaccgtgagctcc

SEQ ID NO: 123 - DNA, 351 bp, synthetic construct
  misc_feature 1..351: note = DNA sequence of IL-6R sdAb C82-111-Fc-011
  source 1..351: mol_type = other DNA
caggtccagctcgtcgagtcaggcggcggcctcgtgcagcctggaggaagtttgaggttgagttgtgcagccagcggtcctacattttccaacctcgacatgggctggtacaggcaggcacccggcaaagggctggagctggtgggcgttatttctcggacaggagggtccacatactacgccgattccgtgaagggcagattcaccatcagcagggacaatgctaagaacaccctgtacctgcagatgaactccttgagggctgaggacaccgcagtgtactactgcaagaccgaggtgcccttttctgacgattactggggacagggcacactggtgaccgtgagctcc

SEQ ID NO: 124 - DNA, 351 bp, synthetic construct
  misc_feature 1..351: note = DNA sequence of IL-6R sdAb C82-111-Fc-013
  source 1..351: mol_type = other DNA
caggtccagctcgtcgagtcaggcggcggcctcgtgcagcctggaggaagtttgaggttgagttgtgcagccagcgtgccgacatttggggatctcgacatgggctggtacaggcaggcacccggcaaagggctggagctggtgggcgttatttctcggacaggagggtccacatactacgccgattccgtgaagggcagattcaccatcagcagggacaatgctaagaacaccctgtacctgcagatgaactccttgagggctgaggacaccgcagtgtactactgcaagaccgaggtgccctttggggacgattactggggacagggcacactggtgaccgtgagctcc

SEQ ID NO: 125 - DNA, 351 bp, synthetic construct
  misc_feature 1..351: note = DNA sequence of IL-6R C82-111-Fc-020
  source 1..351: mol_type = other DNA
caggtccagctcgtcgagtcaggcggcggcctcgtgcagcctggaggaagtttgaggttgagttgtgcagccagcggggatacattttccaacctcgacatgggctggtacaggcaggcacccggcaaagggctggagctggtgggcgttatttctcggacaggagggtccacatactacgccgattccgtgaagggcagattcaccatcagcagggacaatgctaagaacaccctgtacctgcagatgaactccttgagggctgaggacaccgcagtgtactactgcaagaccgaggtgccctgggcggacgattactggggacagggcacactggtgaccgtgagctcc

SEQ ID NO: 126 - DNA, 351 bp, synthetic construct
  misc_feature 1..351: note = DNA sequence of IL-6R sdAb C82-111-Fc-033
  source 1..351: mol_type = other DNA
caggtccagctcgtcgagtcaggcggcggcctcgtgcagcctggaggaagtttgaggttgagttgtgcagccagcgtgccgacattttccgatgtggacatgggctggtacaggcaggcacccggcaaagggctggagctggtgggcgttatttctcggacaggagggtccacatactacgccgattccgtgaagggcagattcaccatcagcagggacaatgctaagaacaccctgtacctgcagatgaactccttgagggctgaggacaccgcagtgtactactgcaagaccgaggtgccctttgctgacgattactggggacagggcacactggtgaccgtgagctcc

SEQ ID NO: 127 - DNA, 351 bp, synthetic construct
  misc_feature 1..351: note = DNA sequence of IL-6R sdAb C82-111-Fc-043
  source 1..351: mol_type = other DNA
caggtccagctcgtcgagtcaggcggcggcctcgtgcagcctggaggaagtttgaggttgagttgtgcagccagcgtgccgtggttttccaacctcgacatgggctggtacaggcaggcacccggcaaagggctggagctggtgggcgttatttctcggacaggagggatgacatactacgccgattccgtgaagggcagattcaccatcagcagggacaatgctaagaacaccctgtacctgcagatgaactccttgagggctgaggacaccgcagtgtactactgcaagaccgaggtgcccggctcggacgattactggggacagggcacactggtgaccgtgagctcc

SEQ ID NO: 128 - DNA, 351 bp, synthetic construct
  misc_feature 1..351: note = DNA sequence of IL-6R sdAb C82-111-Fc-059
  source 1..351: mol_type = other DNA
caggtccagctcgtcgagtcaggcggcggcctcgtgcagcctggaggaagtttgaggttgagttgtgcagccagcggggcgacattttccaacctcgacatgggctggtacaggcaggcacccggcaaagggctggagctggtgggcgttatttctcggacaggagggtccacatactacgccgattccgtgaagggcagattcaccatcagcagggacaatgctaagaacaccctgtacctgcagatgaactccttgagggctgaggacaccgcagtgtactactgcaagaccgaggtgccctttgatgacgattactggggacagggcacactggtgaccgtgagctcc

SEQ ID NO: 129 - DNA, 1818 bp, synthetic construct
  misc_feature 1..1818: note = DNA sequence of EB-105BId1
  source 1..1818: mol_type = other DNA
gaggttcagttggtggagtccgggggaggattggtgcaggctggaggaagtttgaggttgtcctgtgccgccagcggggatgatttgagctccgttgacatgggatggtacaggcaggcacccggaaagggactggagttcgtgggagtgattagcagggggggaggaagcacatactacgtggactccgtgcagggaaggtttacaatctcccgggacaacgctaagaacacagtgtacctgcagatgaacagcctcagggccgaggacacagcagtgtactactgcaaaaccgagattccctattttgacgattactggggccagggcacactggtgaccgttagttccggaggatctggaggaggaagcggaggaggaggaagtggaggtggaggaggttctggaggtggtggatcagacacaggaaggccttttgttgagatgtatagcgagattcccgagatcattcacatgaccgaagggcgggagctggtgattccctgtagggttaccagccccaacatcaccgtgaccctgaagaagtttcccctggacacactgatccccgacggcaaaagaatcatctgggactcccggaagggatttatcatttccaacgccacctacaaggagatcggcctcctgacctgcgaagcaaccgtgaacggccacctctataagaccaactacctgacacaccggcagaccaacacaatcattgacgtggtgctctccccctcccacggaattgagctgtccgttggcgagaaactggtgctgaactgcaccgccaggaccgaactgaacgtgggaatcgacttcaactgggaatacccctccagcaagcaccagcacaagaagctggtgaacagggacctgaaaacccagagcggctccgaaatgaaaaagttcctgtccaccctgaccatcgacggcgtgaccaggtccgaccagggcctgtacacctgcgccgcctcctccggcctgatgaccaagaagaactccaccttcgtgagggtgcacgagaaggacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggaggaggttctggaggtggtggatcagctcagacaaatttcatgcctatggatgatttggagcagaggctgtacgaacagttcatcctgcagcagggcctggaa

SEQ ID NO: 130 - DNA, 1818 bp, synthetic construct
  misc_feature 1..1818: note = DNA sequence of EB-105BId3
  source 1..1818: mol_type = other DNA
gaggttcagttggtggagtccgggggaggattggtgcaggctggaggaagtttgaggttgtcctgtgccgccagcgggggagacttgtcctccgtggacatgggatggtacaggcaggcccctggaaagggattggagtttgtgggggttatttcccggactggagggtccacctattatgttgacagtgtgcagggacggtttaccatttctagggacaacgctaagaacacagtttacctgcagatgaatagcttgagggctgaggacactgctgtgtattattgcaagaccgagattccaggaggagacgactactggggccagggaacactggtgaccgtgagttccggaggatctggaggaggaagcggaggaggaggaagtggaggtggaggaggttctggaggtggtggatcagacacaggaaggccttttgttgagatgtatagcgagattcccgagatcattcacatgaccgaagggcgggagctggtgattccctgtagggttaccagccccaacatcaccgtgaccctgaagaagtttcccctggacacactgatccccgacggcaaaagaatcatctgggactcccggaagggatttatcatttccaacgccacctacaaggagatcggcctcctgacctgcgaagcaaccgtgaacggccacctctataagaccaactacctgacacaccggcagaccaacacaatcattgacgtggtgctctccccctcccacggaattgagctgtccgttggcgagaaactggtgctgaactgcaccgccaggaccgaactgaacgtgggaatcgacttcaactgggaatacccctccagcaagcaccagcacaagaagctggtgaacagggacctgaaaacccagagcggctccgaaatgaaaaagttcctgtccaccctgaccatcgacggcgtgaccaggtccgaccagggcctgtacacctgcgccgcctcctccggcctgatgaccaagaagaactccaccttcgtgagggtgcacgagaaggacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggaggaggttctggaggtggtggatcagctcagacaaatttcatgcctatggatgatttggagcagaggctgtacgaacagttcatcctgcagcagggcctggaa

SEQ ID NO: 131 - DNA, 1812 bp, synthetic construct
  misc_feature 1..1812: note = DNA sequence of EB-105BId2
  source 1..1812: mol_type = other DNA
gctcagacaaatttcatgcctatggatgatttggagcagaggctgtacgaacagttcatcctgcagcagggcctggaaggtggtggcggatcaggtgggggaggctctggtggaggcggtagttccgataccggtcgtccttttgtcgagatgtactccgagatccccgagattattcacatgaccgagggccgcgagctggtgattccttgtcgtgtgacctcccctaacatcaccgttaccctgaagaagtttcccctggatacactgatccctgatggcaagaggattatctgggactccaggaagggattcattatttccaacgccacctacaaggagatcggcctcctgacctgcgaagccacagttaacggccacctctacaagaccaactacctgacacaccggcagaccaacaccatcatcgacgtcgtgctcagcccctcccacggaattgagctgtccgtcggagaaaagctggtgctgaactgcaccgccagaaccgaactgaacgtgggcatcgacttcaactgggagtacccctcctccaaacaccagcacaagaaactcgtcaacagggacctgaaaactcaatctggctccgaaatgaaaaagttcctgtccaccctgaccatcgacggcgtgaccaggtccgaccagggcctgtacacctgcgccgcctcctccggcctgatgaccaagaagaactccaccttcgtgagggtgcacgagaaggacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggtggcggatcaggtgggggaggctctggtggaggcggtagtgaggttcagttggtggagtccgggggaggattggtgcaggctggaggaagtttgaggttgtcctgtgccgccagcggggatgatttgagctccgttgacatgggatggtacaggcaggcacccggaaagggactggagttcgtgggagtgattagcagggggggaggaagcacatactacgtggactccgtgcagggaaggtttacaatctcccgggacaacgctaagaacacagtgtacctgcagatgaacagcctcagggccgaggacacagcagtgtactactgcaaaaccgagattccctattttgacgattactggggccagggcacactggtgaccgttagttcc

SEQ ID NO: 132 - DNA, 1812 bp, synthetic construct
  misc_feature 1..1812: note = DNA sequence of EB-105BId4
  source 1..1812: mol_type = other DNA
gctcagacaaatttcatgcctatggatgatttggagcagaggctgtacgaacagttcatcctgcagcagggcctggaaggtggtggcggatcaggtgggggaggctctggtggaggcggtagttccgataccggtcgtccttttgtcgagatgtactccgagatccccgagattattcacatgaccgagggccgcgagctggtgattccttgtcgtgtgacctcccctaacatcaccgttaccctgaagaagtttcccctggatacactgatccctgatggcaagaggattatctgggactccaggaagggattcattatttccaacgccacctacaaggagatcggcctcctgacctgcgaagccacagttaacggccacctctacaagaccaactacctgacacaccggcagaccaacaccatcatcgacgtcgtgctcagcccctcccacggaattgagctgtccgtcggagaaaagctggtgctgaactgcaccgccagaaccgaactgaacgtgggcatcgacttcaactgggagtacccctcctccaaacaccagcacaagaaactcgtcaacagggacctgaaaactcaatctggctccgaaatgaaaaagttcctgtccaccctgaccatcgacggcgtgaccaggtccgaccagggcctgtacacctgcgccgcctcctccggcctgatgaccaagaagaactccaccttcgtgagggtgcacgagaaggacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggtggcggatcaggtgggggaggctctggtggaggcggtagtgaggttcagttggtggagtccgggggaggattggtgcaggctggaggaagtttgaggttgtcctgtgccgccagcgggggagacttgtcctccgtggacatgggatggtacaggcaggcccctggaaagggattggagtttgtgggggttatttcccggactggagggtccacctattatgttgacagtgtgcagggacggtttaccatttctagggacaacgctaagaacacagtttacctgcagatgaatagcttgagggctgaggacactgctgtgtattattgcaagaccgagattccaggaggagacgactactggggccagggaacactggtgaccgtgagttcc

SEQ ID NO: 133 - DNA, 2097 bp, synthetic construct
  misc_feature 1..2097: note = DNA sequence of EB-105BIe1
  source 1..2097: mol_type = other DNA
gaggttcagttggtggagtccgggggaggattggtgcagcctggaggaagtttgaggttgagctgtgccgcatccgggttgtactttagcagctacgacattgcatgggtgaggcaggcacctggcaagggattggagtttgtgagcgccatcgacgtgagcggagagcatacatactacgctgacagtgtgaagggccggtttaccatctccagggacaacgctaagaacacactgtacctgcagatgaacagcctgagggcagaggacacagcagtgtactactgcaacgcagtgaaatggagcgattacggaccagacaacgactactggggacagggaaccctcgtgaccgtgtctagcggtggtggcggatcaggtgggggaggctctggtggaggcggtagttccgataccggtcgtccttttgtcgagatgtactccgagatccccgagattattcacatgaccgagggccgcgagctggtgattccttgtcgtgtgacctcccctaacatcaccgttaccctgaagaagtttcccctggatacactgatccctgatggcaagaggattatctgggactccaggaagggattcattatttccaacgccacctacaaggagatcggcctcctgacctgcgaagccacagttaacggccacctctacaagaccaactacctgacacaccggcagaccaacaccatcatcgacgtcgtgctcagcccctcccacggaattgagctgtccgtcggagaaaagctggtgctgaactgcaccgccagaaccgaactgaacgtgggcatcgacttcaactgggagtacccctcctccaaacaccagcacaagaaactcgtcaacagggacctgaaaactcaatctggctccgaaatgaaaaagttcctgtccaccctgaccatcgacggcgtgaccaggtccgaccagggcctgtacacctgcgccgcctcctccggcctgatgaccaagaagaactccaccttcgtgagggtgcacgagaaggacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggtggcggatcaggtgggggaggctctggtggaggcggtagtgaggttcagttggtggagtccgggggaggattggtgcaggctggaggaagtttgaggttgtcctgtgccgccagcggggatgatttgagctccgttgacatgggatggtacaggcaggcacccggaaagggactggagttcgtgggagtgattagcagggggggaggaagcacatactacgtggactccgtgcagggaaggtttacaatctcccgggacaacgctaagaacacagtgtacctgcagatgaacagcctcagggccgaggacacagcagtgtactactgcaaaaccgagattccctattttgacgattactggggccagggcacactggtgaccgttagttcc

SEQ ID NO: 134 - DNA, 2097 bp, synthetic construct
  misc_feature 1..2097: note = DNA sequence of EB-105BIe2
  source 1..2097: mol_type = other DNA
gaggttcagttggtggagtccgggggaggattggtgcaggctggaggaagtttgaggttgtcctgtgccgccagcggggatgatttgagctccgttgacatgggatggtacaggcaggcacccggaaagggactggagttcgtgggagtgattagcagggggggaggaagcacatactacgtggactccgtgcagggaaggtttacaatctcccgggacaacgctaagaacacagtgtacctgcagatgaacagcctcagggccgaggacacagcagtgtactactgcaaaaccgagattccctattttgacgattactggggccagggcacactggtgaccgttagttccggtggtggcggatcaggtgggggaggctctggtggaggcggtagttccgataccggtcgtccttttgtcgagatgtactccgagatccccgagattattcacatgaccgagggccgcgagctggtgattccttgtcgtgtgacctcccctaacatcaccgttaccctgaagaagtttcccctggatacactgatccctgatggcaagaggattatctgggactccaggaagggattcattatttccaacgccacctacaaggagatcggcctcctgacctgcgaagccacagttaacggccacctctacaagaccaactacctgacacaccggcagaccaacaccatcatcgacgtcgtgctcagcccctcccacggaattgagctgtccgtcggagaaaagctggtgctgaactgcaccgccagaaccgaactgaacgtgggcatcgacttcaactgggagtacccctcctccaaacaccagcacaagaaactcgtcaacagggacctgaaaactcaatctggctccgaaatgaaaaagttcctgtccaccctgaccatcgacggcgtgaccaggtccgaccagggcctgtacacctgcgccgcctcctccggcctgatgaccaagaagaactccaccttcgtgagggtgcacgagaaggacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggtggcggatcaggtgggggaggctctggtggaggcggtagtgaggttcagttggtggagtccgggggaggattggtgcagcctggaggaagtttgaggttgagctgtgccgcatccgggttgtactttagcagctacgacattgcatgggtgaggcaggcacctggcaagggattggagtttgtgagcgccatcgacgtgagcggagagcatacatactacgctgacagtgtgaagggccggtttaccatctccagggacaacgctaagaacacactgtacctgcagatgaacagcctgagggcagaggacacagcagtgtactactgcaacgcagtgaaatggagcgattacggaccagacaacgactactggggacagggaaccctcgtgaccgtgtctagc

SEQ ID NO: 135 - DNA, 18 bp, synthetic construct
  misc_feature 1..18: note = DNA sequence of CDR1 of Tocilizumab HC
  source 1..18: mol_type = other DNA
agcgaccacgcctggagc

SEQ ID NO: 136 - DNA, 48 bp, synthetic construct
  misc_feature 1..48: note = DNA sequence of CDR2 of Tocilizumab HC
  source 1..48: mol_type = other DNA
tacatcagctacagcggcatcaccacctacaaccccagcctgaagagc

SEQ ID NO: 137 - DNA, 24 bp, synthetic construct
  misc_feature 1..24: note = DNA sequence of CDR3 of Tocilizumab HC
  source 1..24: mol_type = other DNA
gccaggaccaccgccatggactac

SEQ ID NO: 138 - DNA, 21 bp, synthetic construct
  misc_feature 1..21: note = DNA sequence of CDR1 of Tocilizumab LC
  source 1..21: mol_type = other DNA
tacaccagcaggctgcacagc

SEQ ID NO: 139 - DNA, 33 bp, synthetic construct
  misc_feature 1..33: note = DNA sequence of CDR2 of Tocilizumab LC
  source 1..33: mol_type = other DNA
agggccagccaggacatcagcagctacctgaac

SEQ ID NO: 140 - DNA, 27 bp, synthetic construct
  misc_feature 1..27: note = DNA sequence of CDR3 of Tocilizumab LC
  source 1..27: mol_type = other DNA
cagcagggcaacaccctgccctacacc